(19) United States
(12) Patent Application Publication
(10) Pub. No.: US 2023/0203533 A1
(43) Pub. Date: Jun. 29, 2023

(54) MACHINE GUIDED DIRECTED EVOLUTION OF RAAV COMBINATORIAL CAPSID LIBRARIES

(71) Applicant: University of Florida Research Foundation, Incorporated, Gainesville, FL (US)

(72) Inventors: Sergei Zolotukhin, Gainesville, FL (US); Oleksandr Kondratov, Gainesville, FL (US)

(73) Assignee: University of Florida Research Foundation, Incorporated, Gainesville, FL (US)

(21) Appl. No.: 17/922,174

(22) PCT Filed: Apr. 28, 2021
(86) PCT No.: PCT/US2021/029749

§ 371 Date: Oct. 28, 2022

Related U.S. Application Data
(60) Provisional application No. 63/016,829, filed on Apr. 28, 2020.

(51) Int. Cl. C12N 15/86; C40B 10/00

(52) U.S. Cl. CPC C12N 15/86; C40B 10/00; C12N 2710/14143; C12N 2750/14151; C12N 2750/14143

(57) ABSTRACT

Provided herein are methods and compositions useful in the screening and production of recombinant AAV (rAAV) that have structural fitness and potency in insect cells and mammalian cells. The disclosure provides a directed evolution system for generating and selecting rAAV variants based on their Kozak sequences. In some embodiments, methods disclosed herein comprise the use of modified Kozak sequences to express AAV2 VP1 proteins in amounts that are useful for producing rAAV particles having high infectivities and/or transduction efficiencies. In some embodiments, methods of producing, and methods of screening, rAAV are provided that comprise the steps of infecting insect cells by administering rAAV to these cells, recovering rAAV-integrated DNA from these insect cells, and transfecting recovered rAAV into mammalian cells. Also provided herein are nucleic acids comprising nucleotide sequences encoding novel Kozak sequences. Further provided herein are mammalian cells, baculovirus particles and insect cells comprising these nucleic acids.

### RELATED APPLICATIONS

This application claims the benefit of the filing date of U.S. Provisional Application No. 63/016,829, filed Apr. 28, 2020, the entire contents of which are incorporated by reference.

### GOVERNMENT SUPPORT

This invention was made with government support under Grant No. R01 HL097088 awarded by the National Institutes of Health. The government has certain rights in the invention.

### BACKGROUND OF THE INVENTION

Adeno-associated virus (AAV) has emerged as one of the most promising viral vectors for human gene therapy. Recombinant adeno-associated virus (rAAV) is being evaluated in gene therapy clinical trials for hemophilia B, malignant melanoma, cystic fibrosis, and other diseases. While large scale production of rAAV using mammalian cell culture systems has been historically problematic, AAV production systems using insect cells have been shown to be a viable alternative. However, the different cellular environment has provided challenges with regard to viral particle assembly of some AAV serotypes.

The widespread successful use of rAAV in gene therapy has driven the demand for high-yield manufacturing methods of vectors with high transduction efficiency. The Baculovirus/Sf9 system is a promising system for scalable high yield production; however, a major drawback to using an invertebrate cell line compared to a mammalian system is generally altered AAV capsid stoichiometry among the VP1, VP2 and VP3 components of the capsid, resulting in lower biological potency.

### SUMMARY OF THE INVENTION

Provided herein are compositions, methods and host cells related to the production of rAAV in host producer cells (e.g., in insect cells). The biological potency (e.g., infectivity) of rAAV particles partly depends on the virion capsid composition, for example, the stoichiometric ratios of the capsid proteins VP1, VP2, and VP3. Virion capsid assembly is, however, stochastic, making it difficult to engineer capsids with desired VP1:VP2:VP3 ratios.

Some rAAV serotypes (e.g., AAV2, AAV5, AAV8, AAV9, AAV3 and AAV6) manufactured in a heterologous system such as insect cells (e.g., Spodoptera frugiperda 9, or Sf9, cells) are characterized by abnormal VP1 expression levels. Upon viral assembly, the abnormal VP1 expression results in the production of viral particles comprised of improper capsid protein ratios, which hinders the ability of the particles to efficiently transduce cells. Methods of obtaining useful stoichiometric ratios of capsid proteins are complicated by results indicating that an overabundance of VP1 can impair the insect cell's ability to efficiently package the viral particle while low levels of VP1 can result in particles with inefficient transduction. Thus, with respect to insect cell rAAV production systems, it remains a challenge using current techniques to produce increased amounts of infective particles without negatively impacting particle assembly.

Methods and compositions described herein provide recombinant VP1, VP2, or VP3 genes comprising a modified Kozak sequence associated with the respective translation initiation codon of each gene. Recombinant VP1, VP2, or VP3 genes described in the present disclosure are useful to produce infective rAAV particles comprising a gene of interest (e.g., for subsequent research and/or therapeutic uses). In some embodiments, recombinant VP1, VP2, or VP3 genes described in the present disclosure may be used to produce rAAV particles of one or more serotypes in insect cells. In some embodiments, methods and compositions described in the present disclosure may be used to screen and identify recombinant VP1, VP2, or VP3 genes of one or more serotypes that are useful to produce rAAV particles of one or more serotypes in insect cells and/or in other producer cells of interest (e.g., in mammalian cells, such as HEK293T human cells). In some embodiments, one or more recombinant VP1, VP2, or VP3 genes may be useful to produce stable and/or infective rAAV particles that contain recombinant genomes of interest at relatively high frequencies and/or with relatively low rates of mis-packaged nucleic acids.

Previously, it had been shown that manipulation of the translation initiation site (TIS, also known as the Kozak sequence) of the VP1 capsid protein can modulate and improve the VP1:VP2:VP3 ratio, and thus the capsid's biological potency, to match or even exceed rAAV manufactured in a HEK293T cell line. See International Patent Publication No. WO 2017/181162, published Oct. 19, 2017, and US Patent Publication No. 2020/0123572, each of which is incorporated herein by reference. To further develop the approach experimentally, a novel combinatorial capsid library screening protocol was developed for assessing structural and biological fitness of Sf9-manufactured rAAV, irrespective of the capsid serotype, and is herein disclosed. This disclosed approach relies on two directed evolution steps of selection performed in series: 1) structural fitness in Sf9 cells, coupled with 2) transduction efficiency in mammalian cells.

The present disclosure further provides methods for selecting a capsid variant with enhanced infectivity amongst a combinatorial capsid library. These methods comprise the packaging of a combinatorial capsid library in insect Sf9 cells and screening for high infectivity in human Cre-recombinase-expressing mammalian cells. A single directed evolution (DE) selection round, combined with by Next-Generation Sequencing (NGS) and guided by in silico analysis, may identify a group of VP1 Kozak sites that confer enhanced potency properties or characteristics on AAV capsids. Such enhanced potency properties or characteristics include, but are not limited to, high production yield, high multiplicity of infection (MOI), and superior transduction efficiencies. The disclosed selection methods may comprise (1) the use of a combinatorial capsid gene library expressed in Sf9 cells; (2) GFP reporter gene; and (3) Cre-recombinase-controlled flip Lox-sites activated in C12 cells, a mouse hepatoma line. In some embodiments of the disclosed selection methods, capsids with surprisingly improved fitness (e.g., potency characteristics) may be identified by a machine-guided workflow using several consecutive filtering steps. The modified Kozak sequences disclosed herein may be identified from one or more of the DE selection methods provided herein.

Accordingly, in some aspects, the present disclosure provides rAAV particles having higher infectivities (also described herein as “potency” or “fitness”) in insect cells. In various embodiments, the disclosed rAAV particles further have higher transduction efficiencies in mammalian cells. In some aspects, the disclosure provides compositions and methods useful in the production and screening of rAAV particles and libraries thereof. In some aspects, these particles carry a transgene cargo on the nucleic acid vector. In some aspects, the provided particles, and compositions thereof, are used in the treatment of one or more diseases, disorders or conditions, e.g., in a gene therapy regimen.

As would be appreciated by the skilled artisan, the disclosed rAAV particles can be derived from any serotype of AAV, including without limitation any AAV derivative or pseudotype. In some embodiments, these rAAV particles are derived from an AAV2 serotype. In some embodiments, these rAAV particles are derived from an AAV5 serotype. In some embodiments, these rAAV particles are derived from an AAV8 serotype. In some embodiments, these rAAV particles are derived from an AAV-rh serotype, including, without limitation, AAV-rh10 or AAV-rh74. In other aspects, these rAAV particles are derived from an AAV1, AAV2, AAV3, AAV4, AAV5, AAV6, AAV7, AAV8, AAV9, AAV-rh10, AAV-rh74, or AAV-DJ serotype.

In some embodiments, these rAAV particles are not derived from an AAV2 serotype. In some embodiments, these rAAV particles are not derived from an AAV5 serotype. In some embodiments, these rAAV particles are not derived from an AAV8 serotype. In some embodiments, these rAAV particles are not derived from an AAV1, AAV2, AAV3, AAV4, AAV5, AAV6, AAV7, AAV8, AAV9, AAV-rh10, AAV-rh74, or AAV-DJ serotype.

Some embodiments contemplate a nucleic acid (e.g., a nucleic acid molecule such as a DNA molecule) comprising a nucleotide sequence encoding a modified Kozak sequence and adeno-associated virus (AAV) VP1, VP2, and VP3 capsid proteins. In some embodiments, the modified Kozak sequence is selected from AACGATGCATGGC (SEQ ID NO: 14), CAACATGAATGGC (SEQ ID NO: 16), GATCATGGATGGC (SEQ ID NO: 17), or CGCGATGGATGGC (SEQ ID NO: 22).

In some embodiments, the modified Kozak sequence is AACGATGCATGGC (SEQ ID NO: 14). In some embodiments, the modified Kozak sequence is CAACATGAATGGC (SEQ ID NO: 16). In some embodiments, the modified Kozak sequence is GATCATGGATGGC (SEQ ID NO: 17). In some embodiments, the modified Kozak sequence is CGCGATGGATGGC (SEQ ID NO: 22).

As would be appreciated by the skilled artisan, the disclosed VP1, VP2, and VP3 capsid proteins can be derived from any serotype of AAV, including without limitation any AAV derivative or pseudotype. In some embodiments, the VP1, VP2, and VP3 capsid proteins are derived from an AAV2 serotype. In some embodiments, the VP1, VP2, and/or VP3 capsid proteins are variant AAV2 capsid proteins (e.g., they are encoded by a sequence containing one or more mutations and that encodes a capsid protein having one or more amino acid substitutions relative to a corresponding wild-type capsid protein). In some embodiments, the VP1, VP2, and VP3 capsid proteins are derived from an AAV5 serotype. In some embodiments, the VP1, VP2, and/or VP3 capsid proteins are variant AAV5 capsid proteins (e.g., they are encoded by a sequence containing one or more mutations and that encodes a capsid protein having one or more amino acid substitutions relative to a corresponding wild-type capsid protein). In some embodiments, the VP1, VP2, and VP3 capsid proteins are derived from an AAV8 serotype. In some embodiments, the VP1, VP2, and/or VP3 capsid proteins are variant AAV8 capsid proteins (e.g., they are encoded by a sequence containing one or more mutations and that encodes a capsid protein having one or more amino acid substitutions relative to a corresponding wild-type capsid protein). In some embodiments, the VP1, VP2, and VP3 capsid proteins are derived from an AAV-rh serotype, including, without limitation, AAV-rh10 or AAV-rh74. In some embodiments, the VP1, VP2, and VP3 capsid proteins are derived from an AAV1, AAV2, AAV3, AAV4, AAV5, AAV6, AAV7, AAV8, AAV9, AAV-rh10, AAVrh-74, or AAV-DJ serotype. In some embodiments, the VP1, VP2, and VP3 capsid proteins are not derived from an AAV2 serotype, or a variant thereof. In some embodiments, the VP1, VP2, and VP3 capsid proteins are not derived from an AAV5 serotype, or a variant thereof. In some embodiments, the VP1, VP2, and VP3 capsid proteins are not derived from an AAV8 serotype, or a variant thereof. In some embodiments, the VP1, VP2, and VP3 capsid proteins are not derived from an AAV1, AAV2, AAV3, AAV4, AAV5, AAV6, AAV7, AAV8, AAV9, AAV-rh10, AAV-rh74, or AAV-DJ serotype, or a variant thereof.

In some embodiments, the nucleic acid sequence further comprises a promoter sequence. In some embodiments, the promoter sequence is a polyhedrin (polh) promoter sequence.

In some aspects, the present disclosure provides nucleic acids comprising a nucleotide sequence encoding a modified Kozak sequence. In some embodiments, the modified Kozak sequence comprises the initiation codon for translation of an AAV VP1 capsid protein and nucleotide sequence variations in 1-8 nucleotides (e.g., 1 nucleotide, 2 nucleotides, 3 nucleotides, 4 nucleotides, 5 nucleotides, 6 nucleotides, 7 nucleotides, or 8 nucleotides) immediately upstream of the VP1 translation initiation codon (e.g., ATG) and/or nucleotide sequence variations in 1-2 nucleotides (e.g., 1 nucleotide or 2 nucleotides) immediately downstream of the VP1 translation initiation codon (e.g., ATG). In some embodiments, the disclosed nucleic acids comprise NNNNNN(ATG), wherein (ATG) is the VP1 initiation codon. In some embodiments, the nucleic acids comprise NNNNNN(AUG), wherein (AUG) is the VP1 initiation codon, wherein N represents any nucleotide, e.g., any nucleotide that is different from a naturally occurring nucleotide at that position in a wild-type VP1 gene for an AAV serotype of interest. In some embodiments of the disclosed nucleic acids, the Kozak sequence includes the initiation codon for translation, ATG/AUG; in other embodiments, the Kozak sequence comprises NNNNN, without the initiation codon.

In some aspects, the present disclosure provides nucleic acids comprising a nucleotide sequence encoding a modified Kozak sequence. In some embodiments, the modified Kozak sequence comprises the initiation codon for translation of an AAV VP2 capsid protein and nucleotide sequence variations in 1-8 nucleotides (e.g., 1 nucleotide, 2 nucleotides, 3 nucleotides, 4 nucleotides, 5 nucleotides, 6 nucleotides, 7 nucleotides, or 8 nucleotides) immediately upstream of the VP2 translation initiation codon (e.g., ATG) and/or nucleotide sequence variations in 1-2 nucleotides (e.g., 1 nucleotide or 2 nucleotides) immediately downstream of the VP2 translation initiation codon (e.g., ATG). In some embodiments, the disclosed nucleic acids comprise NNNNNN(ATG), wherein (ATG) is the VP2 initiation codon. In some embodiments, the nucleic acids comprise NNNNNN(AUG), wherein (AUG) is the VP2 initiation codon, wherein N represents any nucleotide, e.g., any nucleotide that is different from a naturally occurring nucleotide at that position in a wild-type VP2 gene for an AAV serotype of interest. In some embodiments of the disclosed nucleic acids, the Kozak sequence includes the initiation codon for translation, ATG (or AUG); in other embodiments, the Kozak sequence comprises NNNNN, without the initiation codon.

In some aspects, the present disclosure provides nucleic acids comprising a nucleotide sequence encoding a modified Kozak sequence. In some embodiments, the modified Kozak sequence comprises the initiation codon for translation of an AAV VP3 capsid protein and nucleotide sequence variations in 1-8 nucleotides (e.g., 1 nucleotide, 2 nucleotides, 3 nucleotides, 4 nucleotides, 5 nucleotides, 6 nucleotides, 7 nucleotides, or 8 nucleotides) immediately upstream of the VP3 translation initiation codon (e.g., ATG) and/or nucleotide sequence variations in 1-2 nucleotides (e.g., 1 nucleotide or 2 nucleotides) immediately downstream of the VP3 translation initiation codon (e.g., ATG). In some embodiments, the disclosed nucleic acids comprise NNNNNN(ATG), wherein (ATG) is the VP3 initiation codon. In some embodiments, the nucleic acids comprise NNNNNN(AUG), wherein (AUG) is the VP3 initiation codon, wherein N represents any nucleotide, e.g., any nucleotide that is different from a naturally occurring nucleotide at that position in a wild-type VP3 gene for an AAV serotype of interest. In some embodiments of the disclosed nucleic acids, the Kozak sequence includes the initiation codon for translation, ATG/AUG; in other embodiments, the Kozak sequence comprises NNNNN, without the initiation codon.

Thus, in some embodiments, the modified Kozak sequence contains an initiation codon for translation of the VP1 capsid protein. In some embodiments, the initiation codon for translation of the VP1 capsid protein is AUG. In some embodiments, the modified Kozak sequence contains an initiation codon for translation of the VP2 capsid protein. In some embodiments, the initiation codon for translation of the VP2 capsid protein is AUG. In some embodiments, the modified Kozak sequence contains an initiation codon for translation of the VP3 capsid protein. In some embodiments, the initiation codon for translation of the VP3 capsid protein is AUG.

In some embodiments, the modified Kozak sequence is AACGATGCATGGC (SEQ ID NO: 14), and the ratio of VP1:VP2:VP3 is not 1:1:1. In some embodiments, the modified Kozak sequence is AACGATGCATGGC (SEQ ID NO: 14), and the ratio of VP1:VP2:VP3 is 1:0.3:9. In some embodiments, the modified Kozak sequence is CAACATGAATGGC (SEQ ID NO: 16), and the ratio of VP1:VP2:VP3 is not 1:1:1. In some embodiments, the modified Kozak sequence is CAACATGAATGGC (SEQ ID NO: 16), and the ratio of VP1:VP2:VP3 is 1:0.3:8. In some embodiments, the modified Kozak sequence is GATCATGGATGGC (SEQ ID NO: 17), and the ratio of VP1:VP2:VP3 is not 1:1:1. In some embodiments, the modified Kozak sequence is GATCATGGATGGC (SEQ ID NO: 17), and the ratio of VP1:VP2:VP3 is 1:0.6:9. In some embodiments, the modified Kozak sequence is CGCGATGGATGGC (SEQ ID NO: 22), and the ratio of VP1:VP2:VP3 is not 1:1:1. In some embodiments, the modified Kozak sequence is CGCGATGGATGGC (SEQ ID NO: 22), and the ratio of VP1:VP2:VP3 is 1:0.4:14.

In some embodiments, the nucleic acid is packaged in a viral particle (e.g., a baculovirus particle). In some embodiments, the disclosure provides an insect cell comprising the nucleic acid. In some embodiments, the nucleic acid is integrated into the genome of the insect cell.

Some embodiments contemplate methods of producing and screening recombinant AAV (rAAV). In some embodiments, this method comprises: (a) transfecting a first insect cell with: (i) a baculovirus comprising the nucleic acid as disclosed herein; (ii) a baculovirus comprising a nucleic acid encoding an AAV Rep protein; and (iii) a baculovirus comprising a nucleic acid comprising two AAV inverted terminal repeat (ITR) nucleotide sequences flanking a nucleic acid encoding an AAV Cap protein operably linked to a promoter sequence; (b) culturing the first insect cell under conditions suitable to produce the rAAV; (c) recovering the rAAV from the insect cell; (d) infecting a mammalian cell with the recovered rAAV; and (e) extracting DNA from the mammalian cell. In exemplary embodiments, nucleic acid encoding an AAV Cap protein contains one of a plurality of different recombinant capsid genes, wherein each capsid gene has a different modified Kozak sequence surrounding a VP1 translation initiation codon, is placed between the ITR sequences. This capsid gene may further be linked to one or more lox sequences, such as in the head-to-toe orientation. Such methods of screening uniquely allow for extraction and subsequent sequencing of the modified Kozak sequences associated with capsid proteins having enhanced potency following infection of these particles from mammalian cells, as in step (e). In some embodiments, the VP1 translation initiation codon is an ATG/AUG codon. In some embodiments, the rAAV particles produced by the mammalian cells are evaluated by assaying their relative VP1/VP2/VP3 content, their yield, their DNA content, their infectivity, or a combination of two or more thereof.

In some embodiments, this method further comprises (f) amplifying Kozak regions from the extracted DNA; and (g) subcloning the amplified Kozak regions into an AAV plasmid. In some embodiments, this method still further comprises (h) transfecting a second insect cell with a baculovirus comprising the AAV plasmid of step (g).

In some embodiments, the multiplicity of infection (MOI) of step (a) is about 2, about 3, about 4, about 5, about 10, or about 20. In some embodiments, the MOI of step (a) is about 3. In some embodiments, the MOI of step (d) is about 1,000.

Provided herein are methods of producing rAAV particles of any serotype comprising VP1, VP2, and/or VP3 proteins encoded by recombinant nucleic acids having one or more of the engineered Kozak sequences described in this application. Such nucleic acids can be provided to a host cell along with nucleic acids comprising a recombinant rAAV genome (e.g., comprising a gene of interest flanked by ITRs) in order to produce, in the host cell (e.g., a bacterial cell, an insect cell, or a mammalian cell), rAAV particles comprising the rAAV genome.

In some aspects, these methods of producing comprise a step of contacting an insect cell with a baculovirus comprising a capsid gene containing any of the nucleic acids (modified Kozak sequences) provided herein. The insect cell may be an Sf9 cell. In some embodiments of these methods of production, the methods comprise a step of contacting a mammalian cell with a plasmid comprising a capsid gene containing any of the modified Kozak sequences provided herein. The mammalian cell may be a human cell, such as an HEK293T cell. In some embodiments, the rAAV particles produced according to any of these methods contain an rAAV vector comprising a transgene encoding a protein or polypeptide of interest, flanked between ITR sequences, and optionally operably controlled by a promoter and/or an enhancer. In some embodiments, a multiplicity of infection (MOI) of the disclosed methods of screening is contemplated wherein the MOI is about 2, about 3, about 4, about 5, about 10, or about 20. In some embodiments, the MOI of step (a) is about 3. In some embodiments, the multiplicity of infection (MOI) of step (d) is about 1,000.

The transgene encoding the protein or polypeptide of interest may be, e.g., a polypeptide or protein of interest provided in Table 1. The sequences of the polypeptide or protein of interest may be obtained, e.g., using the non-limiting National Center for Biotechnology Information (NCBI) Protein IDs or SEQ ID NOs from patent applications as provided in Table 1.

Other exemplary polypeptides or proteins of interest include clotting factors, globins (e.g., human β-globin and human γ-globin), adrenergic agonists, anti-apoptosis factors, apoptosis inhibitors, cytokine receptors, cytokines, cytotoxins, erythropoietic agents, glutamic acid decarboxylases, glycoproteins, growth factors, growth factor receptors, hormones, hormone receptors, interferons, interleukins, interleukin receptors, kinases, kinase inhibitors, nerve growth factors, netrins, neuroactive peptides, neuroactive peptide receptors, neurogenic factors, neurogenic factor receptors, neuropilins, neurotrophic factors, neurotrophins, neurotrophin receptors, N-methyl-D-aspartate antagonists, plexins, proteases, protease inhibitors, protein decarboxylases, protein kinases, protein kinase inhibitors, proteolytic proteins, proteolytic protein inhibitors, semaphoring, semaphorin receptors, serotonin transport proteins, serotonin uptake inhibitors, serotonin receptors, serpins, serpin receptors, and tumor suppressors. In some embodiments, the polypeptide or protein of interest is a human protein or polypeptide.

In some embodiments, the first and/or second insect cell is an Sf9 cell. In some embodiments, the mammalian cell is an HEK293T cell.

Some embodiments contemplate an rAAV particle comprising a capsid protein encoded by the nucleic acid as disclosed and/or described herein. In some embodiments, the rAAV is derived from an AAV2 serotype. In some embodiments, the rAAV is derived from an AAV1, AAV2, AAV3, AAV4, AAV5, AAV6, AAV7, AAV8, AAV9, AAV-rh10, AAV-rh74, or AAV-DJ serotype.

Some embodiments contemplate a mammalian cell comprising the nucleic acid as disclosed and/or described herein. In some aspects, a host cell, such as a mammalian host cell, comprising the rAAV particle as disclosed and/or described herein is provided.

Further provided herein are methods of treating a subject suffering from a disease, disorder or condition comprising administering to the subject any of the disclosed rAAV particles or mammalian cells comprising rAAV particles as disclosed herein. In some embodiments, the subject is a human.

Some embodiments of the present disclosure describe a method of identifying certain Kozak sequences that alter the ratios of VP1, VP2, and VP3 in insect cells by modifying the translation thereof. In some embodiments, the identified Kozak sequences are integrated into a library comprising sequences specific to certain AAV plasmids. In some embodiments, the plasmid library comprises AAV1, AAV2, AAV3, AAV4, AAV5, AAV6, AAV7, AAV8, AAV9, AAV-rh10, AAV-rh74, or AAV-DJ serotypes. In some embodiments, the library comprises plasmids comprising the sequences set forth in Tables 2 or 3. In some embodiments, the library can be screened to identify Kozak sequences of interest. In some embodiments, the screening leads to the identification of certain Kozak sequences that lead to the translation of specific ratios of VP1:VP2:VP3, as those ratios are described herein. In some embodiments, the specific Kozak sequences are those described in Table 2. In some embodiments, the specific Kozak sequences are those described in Table 3. In some embodiments, the specific Kozak sequences are those described in Table 4. In some embodiments, each plasmid library has greater than 100× clonal coverage (e.g., greater than 100× copies of each Kozak variant in the plasmid pool). In some embodiments, one or more Kozak sequences are selected that alter the VP1:VP2:VP3 ratio such that the rAAV produced using the methods described herein in insect (e.g., Sf9) cells display characteristics of potency (e.g., transduction efficiency, infectivity, yield, packing, etc.) that are substantially equivalent to those displayed by rAAV produced in mammalian (e.g., HEK293T) cells using the same AAV serotype and wild-type Kozak sequence. In some embodiments, the potency characteristics are equivalent, or roughly identical, to those exhibited by rAAV produced in mammalian (e.g., HEK293T) cells.

Thus, some embodiments contemplate a method of producing an rAAV particle, wherein the rAAV particle is produced in insect cells, wherein one or more Kozak sequences are modified to alter the translation ratio of VP1:VP2:VP3, and wherein the rAAV produced in insect cells display characteristics that are equivalent to an rAAV produced in mammalian cells using the same AAV serotype and the wild-type Kozak sequence.

These and other aspects are described in more detail in the following description and accompanying examples and figures.

### BRIEF DESCRIPTION OF THE DRAWINGS

The following drawings form part of the present specification and are included to demonstrate certain aspects of the present invention. The invention may be better understood by reference to the following description taken in conjunction with the accompanying drawings, in which like reference numerals identify like elements, and in which:

FIG. 1 illustrates a non-limiting design of a capsid helper nucleotide encoding VP1, VP2, and VP3, and the encoded capsid proteins.

FIGS. 2A-2C show Western blot analyses of various AAV serotypes packaged in Sf9 cells. FIG. 2A depicts an example of a Western blotting analysis of rAAV5 packaged in Sf9 cells. FIG. 2B depicts an example of a Western blotting analysis of rAAV8 packaged in Sf9 cells. FIG. 2C depicts an example of a Western blotting analysis of rAAV9 packaged in Sf9 cells.

FIG. 3 depicts a non-limiting nucleotide sequence of a consensus-attenuated Kozak element upstream of the VP1 capsid gene designed for expression in insect cells.

FIGS. 4A-4D depict non-limiting examples of capsid protein compositions of rAAV particles produced in Sf9 cells. FIG. 4A illustrates a non-limiting example of a design of rep and cap gene expression cassettes. FIG. 4B shows an example of a direct correlation of rAAV5 VP1 protein expression and its relative VP1 Kozak translation initiation site (TIS) efficiency. Western blotting analysis of capsid proteins isolated from ten separate cell lines incorporating stably integrated cap expression cassettes is shown. The relative TIS efficiencies (%) for each capsid VP1 gene construct are shown below the respective lane. FIG. 4C shows examples of capsid protein compositions of rAAV5 purified from HEK293 and Sf9 cells. SDS-protein gel analysis of double iodixanol-purified rAAV5-GFP, directly visualized with shortwave UV photoactivation (stain-free technology, Bio-Rad), is shown. (*) denotes a slower migrating band often observed in rAAV5 samples purified from HEK293 cells, which was excluded from VP2 quantification analysis. FIG. 4D shows examples of capsid protein compositions of rAAV9 purified from HEK293 and Sf9 cells. Analysis is the same as in panel FIG. 4C.

FIGS. 5A-5D depict MALDI-TOF analysis of the AAV5 VP1, VP2, and VP3 capsid protein stoichiometry. FIG. 5A shows an amino acid sequence of the AAV5 capsid (SEQ ID NO: 43) with VP1 unique N-termini marked by a dashed line, unique VP2 marked by a dot-dashed line, and common VP3 C-termini marked by a black line. The downward arrows indicate the respective proteins. Tryptic peptides selected for MS analysis are underlined and shown below next to their respective observed masses. (SEQ ID NOS: 44-46 from top to bottom). FIG. 5B shows a MALDI-TOF-MS spectrum of all tryptic peptides of rAAV5 digested in H₂¹⁸O. The circled peptide is one representative out of three analyzed. (SEQ ID NO: 50). FIG. 5C shows two overlaid MALDI-TOF MS spectra of the same tryptic peptide TWVLPSYNNHQYR (SEQ ID NO: 50) originating from the VP1 gel band digested with trypsin prepared in ¹⁶O water, or from the VP3 gel band digested with trypsin prepared in ¹⁸O water. ¹⁸O water incorporates two ¹⁸O atoms on the C-terminus of the peptide thus shifting the mass by 4 atomic mass units (amu). These digestion products were spotted/analyzed separately, and the spectra was overlaid to show the complete incorporation of two ¹⁸O into the VP3 peptide. FIG. 5D shows isotopic “fingers” of the same peptide derived from VP1 or VP3 after the digestion products were mixed at 1:1 ratios to calculate the relative content.

FIGS. 6A-6F depict Nanoparticle Tracking Analysis of sizes and titers of rAAV9 (FIGS. 6A-6B), or rAAV5 (FIGS. 6C-6E) manufactured in HEK293 cells (FIGS. 6A, 6D), or Sf9 cells (FIGS. 6B, 6E). FIGS. 6A, 6B, 6D, and 6E show a graphic representation of Finite Track Length Adjustment (FTLA) algorithm. An average of three independent video captures of rAAV/nano-gold particle complexes, each recorded for 30 sec for each sample, is shown. The numbers next to the peaks show the calculated rAAV/nano-gold particle complex sizes. The smaller peaks of larger diameters represent aggregated dimers, and trimers of rAAV particles. FIGS. 6C and 6F show the calculated ratios of full/total particles for each preparation. Calculated ratios of DNA-containing vs. total number of AAV particles in the respective viral stocks are shown.

FIG. 7 depicts scanned profiles of rAAV5 preparations separated by SDS-PAGE electrophoresis (see FIG. 4C). Shaded areas indicate areas-under-the-curves (AUCs) drawn for quantifications. (*) indicates the peak, which was excluded from the analysis.

FIG. 8 depicts a summary of AAV biology. FIG. 8 includes a schematic depicting the VP1, VP2, and VP3 AAV genome, and the method of AAV viral entry into the cell, as well as results of variant SDS-Page.

FIG. 9 depicts the first generation of baculovirus system-mediated production of rAAV in insect cells (SEQ ID NO: 47).

FIGS. 10A-10C depict improvement of rAAV yield with the use of insect Rep/Cap stable cell lines.

FIGS. 11A-11D depict additional improvements of rAAV yield with use of insect Rep/Cap stable cell lines.

FIG. 12 is a vector map of the Lox-Cap-Koz-GFP library. The crucial elements Lox66-Lox71 (which participate during Cre-mediated screening), Kozak (nucleotide library in front of ATG and wild type second amino acid: NNNN NNNN ATG GC (SEQ ID NO: 48)), EGFP expression cassette (makes a second layer of selection: enrichment of transduced cells), and HR2-distal enhancer (allows for expression of Cap protein for assembly of AAV library using a very compact cassette with a short Polh promoter) are shown.

FIG. 13 depicts rAAV composition-assembly theory and directed evolution, and is a theoretical prediction of the Infectivity/Packaging relationship.

FIG. 14 depicts the workflow of the directed evolution protocol of rAAV combinatorial capsid libraries in insect Sf9 cells. The workflow is described as follows: (i) a cell line was established that expresses Cre under Tet/Dox regulation (based on C12 (Mus musculus) cells); (ii) the virus assembled by Cap expression from vector shown in FIG. 12 was used; (iii) the virus was added to the cells produced in (i), and the cells were treated by Dox for Cre activation and co-transduced by Ad5 (MOI 5) after 48 h; (iv) during the second layer of selection, the cells were sorted to enrich GFP-position cells (transduced cells). From these selected cells, DNA was isolated and amplified with primers against Cre-converted sequences; (v) quantitative NGS allowing for deep coverage of each variant of Kozak was performed in each library (14 million reads for plasmid library, 4 million for viral, and 1-2 million per sorted library); (vi) bioinformatics were performed to de-barcode frequency statistics, calculate enrichments for packaging and infectivity, sort, assess variants' stability, and select top candidates; and (vii) routine lab methods confirmed the selected variants. Pre-screening was performed using qPCR/transduction assay and Western blot on crude lysate after transient expression of Kozak variants in Sf9 cells, and 4 candidate sequences were picked. Selective screening was performed, including iodixanol purification of the 4 selected candidates with subsequent qPCR titering and staining in gel.

FIG. 15 depicts the library design serotype and sequences for LCL2, LCL6 and MCL2 (SEQ ID NOS: 48 and 49).

FIG. 16 depicts the calculations used to define the efficiencies of “packaging” and “infectivity”.

FIG. 17 depicts a transduction/selection scheme for an AAV2 library.

FIG. 18 depicts the packaging of MCL2 and LCL2. The position of each dot is dependent on the sequence: eight NNNN (vertical) NNNN (horizontal) sequences a form fractal-like coding structure. Since the position of the nucleotide is tightly related to the sequence, the same profile is observed among neighboring sequences, especially for the NNNNNNNNATGCG (SEQ ID NO: 48) library. Blue (B) indicates low packaging. Yellow (Y) to red (R) indicates higher packaging, ranging from higher (Y) to highest (R) packaging. (SEQ ID NOs: 49 and 48, from left to right)

FIG. 19 depicts LCL2 infectivity (GFP-sorted, biological replicate). The fractal coding of DNA sequences for Assay #1 and #2 are shown.

FIG. 20 depicts the VP ratio of Kozak variants relative to VP1. Results of the search of the variant in the MCL library with a design like NNN NNN ATG NN (SEQ ID NO: 49) are shown. Despite a good VP1:VP2:VP3 proportion for MCL2-1 (relative to the standard HEK293 ratio), the MCL2-1 was found to be only 50% efficient, relative to HEK293. The results showed that, despite a proper VP1:VP2:VP3 ratio, altering the second amino acid can jeopardize the bioactivity of variants. Of note, the MCL2-2 variant has 0% activity due to the absence of VP1.

FIG. 21 depicts the read coverage of the Plasmid and Viral libraries, and indicates a transition to packaging for the AAV2 (LCL2) library.

FIG. 22 depicts a LCL2 64 k packaging vs. infectivity plot.

FIG. 23 depicts results of coverage of the GFP-sorted library, which was Cre-recombinated with MOI100. The experiment was performed in duplicates (starting from vials of cells, measuring transductions, performing PCR, performing NGS). The X-axis shows Kozak variants 1 through 65,536, the Y-axis shows read count (top) or infectivity (bottom).

FIG. 24 depicts the results of coverage of the non-GFP-selected (mixed) library, which was Cre-recombinated with MOI100. The experiment was done in duplicates (starting from vials of cells, measuring transductions, performing PCR, performing NGS). The X-axis shows Kozak variants 1 through 65,536, the Y-axis shows read count (top) or infectivity (bottom).

FIG. 25 depicts the results of coverage of the non-GFP-selected (mixed) library, which was Cre-recombinated with ultra-low MOI. The experiment was done in duplicates (starting from vials of cells, measuring transductions, performing PCR, performing NGS). The X-axis shows Kozak variants 1 through 65,536, the Y-axis shows read count (top) or infectivity (bottom).

FIG. 26 depicts the fitness of sorted (LCL2), and is a correlation between two biological replicates.

FIG. 27 depicts the fitness of mixed (LCL2), and is a correlation between two biological replicates.

FIG. 28 depicts the fitness of ultra-low MOI (LCL2), and is a correlation between two biological replicates.

FIG. 29 depicts the overlapping of the pre-selected Kozak sequences for GFP Sorted-I and Sorted-II replicates.

FIG. 30 depicts the overlapping of pre-selected Kozak sequences for non-GFP-sorted, ultra-low MOI-I and ultra-low-MOI-II replicates.

FIG. 31 depicts the overlapping of pre-selected Kozak sequences for GFP-sorted and ultra-low MOI for LCL2.

FIG. 32 depicts results for pre-sorted GFP for LCL2 library. Packaging (X-axis) is plotted versus Infectivity (Y-axis).

FIG. 33 depicts highlighted consensus-families (packaging) for LCL2.

FIG. 34 depicts a stain-free gel of assembled rAAV with selected Kozak variants. The variants were selected using the 1-step machine-guided directed evolution method.

FIG. 35 depicts UV-microscope pictures of C12 cells infected by either the selected AAV or HEK293 AAV.

FIG. 36 depicts FACS counting on BD Accuri C6. C12 cells were infected by either the selected AAV or HEK293 AAV, MOI 10,000, and were co-infected by Ad5 (MOI 5). After 48 h, the positive cells were counted. The number of positive cells represents infectivity. This experiment was done in triplicates.

FIG. 37 depicts the correlation between the bioinformatics MG-DE units of the method disclosed herein (e.g., infectivity or packaging/yield) and real experimental data (e.g., infectivity or qPCR titer).

FIG. 38 depicts rAAV composition-assembly theory and directed evolution, and shows a theoretical prediction of the Infectivity/Packaging relationship.

FIG. 39 depicts the packaging-infectivity relationship seen during the practice of the present method.

FIG. 40 depicts the packaging-infectivity relationship seen during the practice of the present method (approximate).

FIG. 41 depicts the LCL6 plasmid distribution.

FIG. 42 depicts the LCL6 viral distribution.

FIG. 43 depicts the LCL6 infectivity distribution.

FIG. 44 depicts preliminary experiments that were performed for selected LCL6 Kozak variants.

FIG. 45 depicts MCL2 trial sequencing data for plasmid distribution.

FIG. 46 depicts MCL2 trial sequencing data for viral distribution.

FIG. 47 depicts MCL2 trial sequencing data for packaging distribution.

FIG. 48 depicts the final AAV library, produced using the methods disclosed herein (SEQ ID NOS: 33-42 from top to bottom).

FIGS. 49A and 49B depict a representative correlation of packaging between AAV7 and AAV8 libraries (FIG. 49A) and AAV6 and AAV7 libraries (FIG. 49B).

FIGS. 50A and 50B depict PCA analysis of infectivity for AAV1, AAV3, AAV6, AAV7, AAV8, AAV9, AAVrh10 and AAV-DJ libraries. FIG. 50A depicts #31400 (circled), the selected variant (asterisk), and the spiked reference (#70000). FIG. 50B depicts PCA analysis of infectivity for AAV2, AAV4, AAV5, and AAV6.

FIGS. 51A-51P depict representations of packaging-Kozak distribution and packaging-infectivity rates for AAV1, AAV3, AAV6, AAV7, AAV8, AAV9, AAVrh10, and AAV-DJ libraries.

### DETAILED DESCRIPTION OF THE INVENTION

Provided herein are compositions and methods related to the production of rAAV in host producer cells (e.g., in insect cells). The biological potency (e.g., infectivity) of rAAV particles partly depends on the virion capsid composition, for example, the stoichiometric ratios of the capsid proteins VP1, VP2, and VP3. Virion capsid assembly is, however, stochastic, making it difficult to engineer capsids with desired VP1:VP2:VP3 ratios.

Some rAAV serotypes (e.g., AAV2, AAV5, AAV8, AAV9, AAV3 and AAV6) manufactured in a heterologous system such as insect cells (e.g., Spodoptera frugiperda 9, or Sf9, cells) are characterized by abnormal VP1 expression levels. Upon viral assembly, the abnormal VP1 expression results in the production of viral particles comprised of improper capsid protein ratios, which hinders the ability of the particles to efficiently transduce cells. Methods of obtaining useful stoichiometric ratios of capsid proteins are complicated by results indicating that an overabundance of VP1 can impair the insect cell's ability to efficiently package the viral particle while low levels of VP1 can result in particles with inefficient transduction. Thus, with respect to insect cell rAAV production systems, it remains a challenge using current techniques to produce increased amounts of infective particles without negatively impacting particle assembly.

Methods and compositions described herein provide recombinant VP1, VP2, or VP3 genes comprising a modified Kozak sequence associated with the respective translation initiation codon. Recombinant VP1, VP2, or VP3 genes described in the present disclosure are useful to produce infective rAAV particles comprising a gene of interest (e.g., for subsequent research and/or therapeutic uses). In some embodiments, recombinant VP1, VP2, or VP3 genes described in the present disclosure may be used to produce rAAV particles of one or more serotypes in insect cells. In some embodiments, methods and compositions described in the present disclosure may be used to screen and identify recombinant VP1, VP2, or VP3 genes of one or more serotypes that are useful to produce rAAV particles of one or more serotypes in insect cells and/or in other producer cells of interest (e.g., in mammalian cells, such as HEK293T human cells). In some embodiments, one or more recombinant VP1, VP2, or VP3 genes may be useful to produce stable and/or infective rAAV particles that contain recombinant genomes of interest at relatively high frequencies and/or with relatively low rates of mis-packaged nucleic acids.

Previously, it had been shown that manipulation of the translation initiation site (TIS, also known as Kozak sequence) of the VP1 capsid protein can modulate and improve the VP1:VP2:VP3 ratio, and thus the capsid's biological potency, to match or even exceed rAAV manufactured in the HEK293T cell line. See International Patent Publication No. WO 2017/181162, published Oct. 19, 2017, and US Patent Publication No. 2020/0123572, each of which is incorporated herein by reference. To further develop the approach experimentally, a novel combinatorial capsid library screening protocol was developed for assessing structural and biological fitness of Sf9-manufactured rAAV, irrespective of the capsid serotype and is herein disclosed. This disclosed approach relies on two directed evolution steps of selection performed in series: 1) structural fitness in Sf9 cells, coupled with 2) transduction efficiency in mammalian cells.

Accordingly, in some aspects, the present disclosure provides rAAV particles having higher infectivities (also described herein as “potency” or “fitness”) in insect cells. In various embodiments, the disclosed rAAV particle further have higher transduction efficiencies in mammalian cells. In some aspects, the disclosure provides compositions and methods useful in the production and screening of rAAV particles and libraries thereof.

As would be appreciated by the skilled artisan, the disclosed rAAV particles can be derived from any serotype of AAV, including without limitation any AAV derivative or pseudotype. In some embodiments, these rAAV particles are derived from an AAV2 serotype. In some embodiments, these rAAV particles are derived from an AAV5 serotype. In some embodiments, these rAAV particles are derived from an AAV8 serotype. In some embodiments, these rAAV particles are derived from an AAV-rh serotype, including, without limitation, AAV-rh10 or AAV-rh74. In other aspects, these rAAV particles are derived from an AAV1, AAV2, AAV3, AAV4, AAV5, AAV6, AAV7, AAV8, AAV9, AAV-rh10, AAV-rh74, or AAV-DJ serotype. In some embodiments, these rAAV particles are not derived from an AAV2 serotype. In some embodiments, these rAAV particles are not derived from an AAV5 serotype. In some embodiments, these rAAV particles are not derived from an AAV8 serotype. In some embodiments, these rAAV particles are not derived from an AAV1, AAV2, AAV3, AAV4, AAV5, AAV6, AAV7, AAV8, AAV9, AAV-rh10, AAV-rh74, or AAV-DJ serotype.

In various embodiments, these nucleic acid molecules contain modified Kozak sequences. In some embodiments, any of the disclosed nucleic acid molecules containing a modified Kozak sequence is a DNA molecule. In some embodiments, the nucleic acid molecule containing a modified Kozak sequence is an RNA molecule.

In some embodiments, provided herein are nucleic acid molecules (e.g., DNA or RNA molecules) that are associated with raising the ratio of VP1 relative to VP2 and VP3, and are not associated with raising the ratio of VP2 or VP3 relative to VP1. In some embodiments, the nucleic acid molecules provided herein are engineered to include a modified Kozak sequence associated with the VP1 TIS, but otherwise have unmodified (e.g., wild-type) Kozak sequences associated with the VP2 and VP3 TISs.

Some embodiments contemplate a nucleic acid comprising a nucleotide sequence encoding a modified Kozak sequence and adeno-associated virus (AAV) VP1, VP2, and VP3 capsid proteins. In some embodiments, the modified Kozak sequence is selected from AACGATGCATGGC (SEQ ID NO: 14), CAACATGAATGGC (SEQ ID NO: 16), GATCATGGATGGC (SEQ ID NO: 17), or CGCGATGGATGGC (SEQ ID NO: 22).

In some embodiments, the modified Kozak sequence is AACGATGCATGGC (SEQ ID NO: 14). In some embodiments, the modified Kozak sequence is CAACATGAATGGC (SEQ ID NO: 16). In some embodiments, the modified Kozak sequence is GATCATGGATGGC (SEQ ID NO: 17). In some embodiments, the modified Kozak sequence is CGCGATGGATGGC (SEQ ID NO: 22).

In some embodiments, the modified Kozak sequence is a nucleotide sequence comprising no more than 3 nucleic acid variations relative to a nucleotide sequence selected from any one of SEQ ID NOs: 13-14, 16-17, 22, 51-52, 54, and 56-129. In some embodiments, the modified Kozak sequence is a nucleotide sequence comprising no more than 2 nucleic acid variations relative to a nucleotide sequence selected from any one of SEQ ID NOs: 13-14, 16-17, 22, 51-52, 54, and 56-129. In some embodiments, the modified Kozak sequence is a nucleotide sequence comprising no more than 1 nucleic acid variation relative to a nucleotide sequence selected from any one of SEQ ID NOs: 13-14, 16-17, 22, 51-52, 54, and 56-129. In some embodiments, the modified Kozak sequence is a nucleotide sequence sequence selected from any one of SEQ ID NOs: 13-14, 16-17, 22, 51-52, 54, and 56-129. These variations may comprise nucleotides that have been inserted, deleted, or substituted relative to any one of SEQ ID NOs: 13-14, 16-17, 22, 51-52, 54, and 56-129. In some embodiments, the disclosed nucleic acids contain stretches of 3, 4, 5, 6, 7, 8, 9 or 10 nucleotides in common with any one of SEQ ID NOs: 13-14, 16-17, 22, 51-52, 54, and 56-129. In some embodiments, the nucleic acid sequences comprise truncations at the 5′ or 3′ end relative to any one of SEQ ID NOs: 13-14, 16-17, 22, 51-52, 54, and 56-129.

As would be appreciated by the skilled artisan, the disclosed VP1, VP2, and VP3 capsid proteins can be derived from any serotype of AAV, including without limitation any AAV derivative or pseudotype. In some embodiments, the VP1, VP2, and VP3 capsid proteins are derived from an AAV2 serotype. In some embodiments, the VP1, VP2, and/or VP3 capsid proteins are variant AAV2 capsid proteins (e.g., they are encoded by a sequence containing one or more mutations and that encodes a capsid protein having one or more amino acid substitutions relative to a corresponding wild-type capsid protein). In some embodiments, the VP1, VP2, and VP3 capsid proteins are derived from an AAV5 serotype. In some embodiments, the VP1, VP2, and/or VP3 capsid proteins are variant AAV5 capsid proteins (e.g., they are encoded by a sequence containing one or more mutations and that encodes a capsid protein having one or more amino acid substitutions relative to a corresponding wild-type capsid protein). In some embodiments, the VP1, VP2, and VP3 capsid proteins are derived from an AAV8 serotype. In some embodiments, the VP1, VP2, and/or VP3 capsid proteins are variant AAV8 capsid proteins (e.g., they are encoded by a sequence containing one or more mutations and that encodes a capsid protein having one or more amino acid substitutions relative to a corresponding wild-type capsid protein). In some embodiments, the VP1, VP2, and VP3 capsid proteins are derived from an AAV-rh serotype, including, without limitation, AAV-rh10 or AAV-rh74. In some embodiments, the VP1, VP2, and VP3 capsid proteins are derived from an AAV1, AAV2, AAV3, AAV4, AAV5, AAV6, AAV7, AAV8, AAV9, AAV-rh10, AAV-rh74, or AAV-DJ serotype. In some embodiments, the VP1, VP2, and VP3 capsid proteins are not derived from an AAV2 serotype, or a variant thereof. In some embodiments, the VP1, VP2, and VP3 capsid proteins are not derived from an AAV5 serotype, or a variant thereof. In some embodiments, the VP1, VP2, and VP3 capsid proteins are not derived from an AAV8 serotype, or a variant thereof. In some embodiments, the VP1, VP2, and VP3 capsid proteins are not derived from an AAV1, AAV2, AAV3, AAV4, AAV5, AAV6, AAV7, AAV8, AAV9, AAV-rh10, AAV-rh74, or AAV-DJ serotype, or a variant thereof.

In some embodiments, the nucleic acid sequence further comprises a promoter sequence. In some embodiments, the promoter sequence is a polyhedrin (polh) promoter sequence.

In some aspects, the present disclosure provides nucleic acids comprising a nucleotide sequence encoding a modified Kozak sequence. In some embodiments, the modified Kozak sequence comprises the initiation codon for translation of an AAV VP1 capsid protein and nucleotide sequence variations in 1-8 nucleotides (e.g., 1 nucleotide, 2 nucleotides, 3 nucleotides, 4 nucleotides, 5 nucleotides, 6 nucleotides, 7 nucleotides, or 8 nucleotides) immediately upstream of the VP1 translation initiation codon (e.g., ATG) and/or nucleotide sequence variations in 1-2 nucleotides (e.g., 1 nucleotide or 2 nucleotides) immediately downstream of the VP1 translation initiation codon (e.g., ATG). In some embodiments, the disclosed nucleic acids comprise NNNNNN(ATG), wherein (ATG) is the VP1 initiation codon. In some embodiments, the nucleic acids comprise NNNNNN(AUG), wherein (AUG) is the VP1 initiation codon, wherein N represents any nucleotide, e.g., any nucleotide that is different from a naturally occurring nucleotide at that position in a wild-type VP1 gene for an AAV serotype of interest. In some embodiments of the disclosed nucleic acids, the Kozak sequence includes the initiation codon for translation, ATG/AUG; in other embodiments, the Kozak sequence comprises NNNNN, without the initiation codon.

In some aspects, the present disclosure provides nucleic acids comprising a nucleotide sequence encoding a modified Kozak sequence. In some embodiments, the modified Kozak sequence comprises the initiation codon for translation of an AAV VP2 capsid protein and nucleotide sequence variations in 1-8 nucleotides (e.g., 1 nucleotide, 2 nucleotides, 3 nucleotides, 4 nucleotides, 5 nucleotides, 6 nucleotides, 7 nucleotides, or 8 nucleotides) immediately upstream of the VP2 translation initiation codon (e.g., ATG) and/or nucleotide sequence variations in 1-2 nucleotides (e.g., 1 nucleotide or 2 nucleotides) immediately downstream of the VP2 translation initiation codon (e.g., ATG). In some embodiments, the disclosed nucleic acids comprise NNNNNN(ATG), wherein (ATG) is the VP2 initiation codon. In some embodiments, the nucleic acids comprise NNNNNN(AUG), wherein (AUG) is the VP2 initiation codon, wherein N represents any nucleotide, e.g., any nucleotide that is different from a naturally occurring nucleotide at that position in a wild-type VP2 gene for an AAV serotype of interest. In some embodiments of the disclosed nucleic acids, the Kozak sequence includes the initiation codon for translation, ATG/AUG; in other embodiments, the Kozak sequence comprises NNNNN, without the initiation codon.

In some aspects, the present disclosure provides nucleic acids comprising a nucleotide sequence encoding a modified Kozak sequence. In some embodiments, the modified Kozak sequence comprises the initiation codon for translation of an AAV VP3 capsid protein and nucleotide sequence variations in 1-8 nucleotides (e.g., 1 nucleotide, 2 nucleotides, 3 nucleotides, 4 nucleotides, 5 nucleotides, 6 nucleotides, 7 nucleotides, or 8 nucleotides) immediately upstream of the VP3 translation initiation codon (e.g., ATG) and/or nucleotide sequence variations in 1-2 nucleotides (e.g., 1 nucleotide or 2 nucleotides) immediately downstream of the VP3 translation initiation codon (e.g., ATG). In some embodiments, the disclosed nucleic acids comprise NNNNNN(ATG), wherein (ATG) is the VP3 initiation codon. In some embodiments, the nucleic acids comprise NNNNNN(AUG), wherein (AUG) is the VP3 initiation codon, wherein N represents any nucleotide, e.g., any nucleotide that is different from a naturally occurring nucleotide at that position in a wild-type VP3 gene for an AAV serotype of interest. In some embodiments of the disclosed nucleic acids, the Kozak sequence includes the initiation codon for translation, ATG/AUG; in other embodiments, the Kozak sequence comprises NNNNN, without the initiation codon.

Thus, in some embodiments, the modified Kozak sequence contains an initiation codon for translation of the VP1 capsid protein. In some embodiments, the initiation codon for translation of the VP1 capsid protein is AUG. In some embodiments, the modified Kozak sequence contains an initiation codon for translation of the VP2 capsid protein. In some embodiments, the initiation codon for translation of the VP2 capsid protein is AUG. In some embodiments, the modified Kozak sequence contains an initiation codon for translation of the VP3 capsid protein. In some embodiments, the initiation codon for translation of the VP3 capsid protein is AUG.

In some embodiments, the modified Kozak sequence is AACGATGCATGGC (SEQ ID NO: 14), and the ratio of VP1:VP2:VP3 is not 1:1:1. In some embodiments, the modified Kozak sequence is AACGATGCATGGC (SEQ ID NO: 14), and the ratio of VP1:VP2:VP3 is 1:0.3:9. In some embodiments, the modified Kozak sequence is CAACATGAATGGC (SEQ ID NO: 16), and the ratio of VP1:VP2:VP3 is not 1:1:1. In some embodiments, the modified Kozak sequence is CAACATGAATGGC (SEQ ID NO: 16), and the ratio of VP1:VP2:VP3 is 1:0.3:8. In some embodiments, the modified Kozak sequence is GATCATGGATGGC (SEQ ID NO: 17), and the ratio of VP1:VP2:VP3 is not 1:1:1. In some embodiments, the modified Kozak sequence is GATCATGGATGGC (SEQ ID NO: 17), and the ratio of VP1:VP2:VP3 is 1:0.6:9. In some embodiments, the modified Kozak sequence is CGCGATGGATGGC (SEQ ID NO: 22), and the ratio of VP1:VP2:VP3 is not 1:1:1. In some embodiments, the modified Kozak sequence is CGCGATGGATGGC (SEQ ID NO: 22), and the ratio of VP1:VP2:VP3 is 1:0.4:14.

In some embodiments, the modified Kozak sequence is a nucleotide sequence comprising no more than 3 nucleic acid variations relative to a nucleotide sequence selected from any one of SEQ ID NOs: 13-14, 16-17, 22, 51-52, 54, and 56-129, and the ratio of VP1:VP2:VP3 is not 1:1:1.

In some embodiments, the modified Kozak sequence is a nucleotide sequence comprising no more than 2 nucleic acid variations relative to a nucleotide sequence selected from any one of SEQ ID NOs: 13-14, 16-17, 22, 51-52, 54, and 56-129, and the ratio of VP1:VP2:VP3 is not 1:1:1.

In some embodiments, the modified Kozak sequence is a nucleotide sequence comprising no more than 1 nucleic acid variation relative to a nucleotide sequence selected from any one of SEQ ID NOs: 13-14, 16-17, 22, 51-52, 54, and 56-129, and the ratio of VP1:VP2:VP3 is not 1:1:1.

In some embodiments, the modified Kozak sequence is a nucleotide sequence sequence selected from any one of SEQ ID NOs: 13-14, 16-17, 22, 51-52, 54, and 56-129, and the ratio of VP1:VP2:VP3 is not 1:1:1.

In some embodiments, the nucleic acid is packaged in a viral particle (e.g., a baculovirus particle). In some embodiments, the disclosure provides an insect cell comprising the nucleic acid. In some embodiments, the nucleic acid is integrated into the genome of the insect cell.

Some embodiments contemplate methods of producing recombinant AAV (rAAV). In some embodiments, this method comprises: (a) transfecting a first insect cell with: (i) a baculovirus comprising the nucleic acid as disclosed herein; (ii) a baculovirus comprising a nucleic acid encoding an AAV Rep protein; and (iii) a baculovirus comprising a nucleic acid comprising two AAV inverted terminal repeat (ITR) nucleotide sequences flanking a gene of interest operably linked to a promoter sequence; (b) culturing the first insect cell under conditions suitable to produce the rAAV; and (c) recovering the rAAV from the insect cell.

In certain methods of producing and screening rAAV, provided herein are methods such as that described above wherein, rather than a gene of interest, an AAV Cap protein-encoding sequence is inserted between the ITR nucleotide sequences of (a)(iii), optionally wherein the Cap-encoding sequence is operably linked to a lox recognition sequence, and/or a promoter. Such methods may further comprise: (d) infecting a mammalian cell with the recovered rAAV; and (e) extracting DNA from the mammalian cell. In some embodiments, such methods further comprises (f) amplifying Kozak regions from the extracted DNA; and (g) subcloning the amplified Kozak regions into an AAV plasmid. In some embodiments, this method still further comprises (h) transfecting a second insect cell with a baculovirus comprising the AAV plasmid of step (g).

### Genes of Interest

In some embodiments, a nucleic acid vector (e.g., an rAAV genome that is packaged in an rAAV particle) comprises one or more transgenes (e.g., flanked by AAV ITR sequences), wherein the one or more transgenes comprise a sequence encoding a protein or polypeptide of interest, such as a therapeutic protein provided in Table 1 or described herein.

The transgene encoding the protein or polypeptide of interest may be, e.g., a polypeptide or protein of interest provided in Table 1. The sequences of the polypeptide or protein of interest may be obtained, e.g., using the non-limiting National Center for Biotechnology Information (NCBI) Protein IDs or SEQ ID NOs from patent applications as provided in Table 1.

TABLE 1
Non-limiting examples of proteins or polypeptides of
interest and associated diseases.

| | Non-limiting | Exemplary NCBI |
|---|---|---|
| | Exemplary | Protein IDs or Patent |
| Protein or Polypeptide | diseases | SEQ ID NOs |
| acid alpha-glucosidase | Pompe Disease | NP_000143.2, |
| (GAA) | | NP_001073271.1, |
| | | NP_001073272.1 |
| Methyl CpG binding | Rett syndrome | NP_001104262.1, |
| protein 2 (MECP2) | | NP_004983.1 |
| Aromatic L-amino acid | Parkinson’s disease | NP_000781.1, |
| decarboxylase (AADC) | | NP_001076440.1, |
| | | NP_001229815.1, |
| | | NP_001229816.1, |
| | | NP_001229817.1, |
| | | NP_001229818.1, |
| | | NP_001229819.1 |
| Glial cell-derived | Parkinson’s disease | NP_000505.1, |
| neurotrophic | | NP_001177397.1, |
| factor (GDNF) | | NP_001177398.1, |
| | | NP_001265027.1, |
| | | NP_954701.1 |
| Cystic fibrosis | Cystic fibrosis | NP_000483.3 |
| transmembrane | | |
| conductance regulator (CFTR) | | |
| Tumor necrosis | Arthritis, | SEQ ID NO. 1 of |
| factor receptor | Rheumatoid arthriti | WO2013025079 |
| fused to an antibody | | |
| Fc(TNFR:Fc) | | |
| HIV-1 gag-proΔr | HIV infection | SEQ ID NOs. 1-5 of |
| (tgAAC09) | | WO2006073496 |
| Sarcoglycan alpha, | Muscular | SGCA |
| beta, gamma, | dystrophy | NP_000014.1, |
| delta, epsilon, | | NP_001129169.1 |
| or zeta (SGCA, | | SGCB |
| SGCB, SGCG, | | NP_000223.1 |
| SGCD, SGCE, or | | SGCG |
| SGCZ) | | NP_000222.1 |
| | | SGCD |
| | | NP_000328.2, |
| | | NP_001121681.1, |
| | | NP_758447.1 |
| | | SGCE |
| | | NP_001092870.1, |
| | | NP_001092871.1, |
| | | NP_003910.1 |
| | | SGCZ |
| | | NP_631906.2 |
| Alpha-1-antitrypsin | Hereditary | NP_000286.3, |
| (AAT) | emphysema or | NP_001002235.1, |
| | Alpha-1 -antitrypsin | NP_001002236.1, |
| | Deficiency | NP_001121172.1, |
| | | NP_001121173.1, |
| | | NP_001121174.1, |
| | | NP_001121175.1, |
| | | NP_001121176.1, |
| | | NP_001121177.1, |
| | | NP_001121178.1, |
| | | NP_001121179.1 |
| Glutamate decarboxylase | Parkinson’s disease | NP_000808.2, |
| 1(GAD1) | | NP_038473.2 |
| Glutamate decarboxylase | Parkinson’s disease | NP_000809.1, |
| 2 (GAD2) | | NP_001127838.1 |
| Aspartoacylase (ASPA) | Canavan’s disease | NP_000040.1, |
| | | NP_001121557.1 |
| Nerve growth | Alzheimer’s disease | NP_002497.2 |
| factor (NGF) | | |
| Granulocyte-macrophage | Prostate cancer | NP_000749.2 |
| colonystimulating | | |
| factory (GM-CSF) | | |
| Cluster of | Malignant melanoma | NP_001193853.1, |
| Differentiation 86 (CD8 | | NP_001193854.1, |
| or B7-2) | | NP_008820.3, |
| | | NP_787058.4, |
| | | NP_795711.1 |
| Interleukin 12 (IL-12) | Malignant melanoma | NP_000873.2, |
| | | NP_002178.2 |
| neuropeptide Y (NPY) | Parkinson’s disease, | NP_000896.1 |
| | epilepsy | |
| ATPase, Ca++ | Chronic heart failure | NP_001672.1, |
| transporting, cardia | | NP_733765.1 |
| muscle, slow twitch | | |
| 2 (SERCA2) | | |
| Dystrophin or | Muscular dystrophy | NP_000100.2, |
| Minidystrophin | | NP_003997.1, |
| | | NP_004000.1, |
| | | NP_004001.1, |
| | | NP_004002.2, |
| | | NP_004003.1, |
| | | NP_004004.1, |
| | | NP_004005.1, |
| | | NP_004006.1, |
| | | NP_004007.1, |
| | | NP_004008.1, |
| | | NP_004009.1, |
| | | NP_004010.1, |
| | | NP_004011.2, |
| | | NP_004012.1, |
| | | NP_004013.1, |
| | | NP_004014.1 |
| Ceroid lipofuscinosis | Late infantile neuronal | NP_000382.3 |
| neuronal 2 | ceroidlipofuscinosis or | |
| (CLN2) | Batten’s disease | |
| Neurturin (NRTN) | Parkinson’s disease | NP_004549.1 |
| N-acetylgluco- | Sanfilippo syndrome | NP_000254.2 |
| saminidase, alpha | (MPSIIIB) | |
| (NAGLU) | | |
| Iduronidase, | MPSI-Hurler | NP_000194.2 |
| alpha-1 (IDUA) | | |
| Iduronate | MPSII-Hunter | NP_000193.1, |
| 2-sulfatase (IDS) | | NP_001160022.1, |
| | | NP_006114.1 |
| Glucuronidase, | MPSVII-Sly | NP_000172.2, |
| beta (GUSB) | | NP_001271219.1 |
| Hexosaminidase A, | Tay-Sachs | NP_000511.2 |
| a polypeptide | | |
| (HEXA) | | |
| Retinal pigment | Leber congenital | NP_000320.1 |
| epithelium-specifi | amaurosis | |
| protein 65kDa (RPE65) | | |
| Factor IX (FIX) | Hemophilia B | NP_000124.1 |
| Factor IX, Padua mutant | | |
| Adenine nucleotide | progressive external | NP_001142.2 |
| translocator (ANT-1) | ophthalmoplegia | |
| ApaLI | mitochondrial | YP_007161330.1 |
| | heteroplasmy, | |
| | myoclonic epilepsy | |
| | with ragged | |
| | red fibers (MERRF) | |
| | or mitochondrial | |
| | encephalomyopathy, | |
| | lactic acidosis, and stroke-lik | |
| | episodes (MELAS) | |
| NADH ubiquinone | Leber hereditary | YP_003024035.1 |
| oxidoreductase subunit 4 (ND4) | Optic | |
| very long-acyl-CoA | very long-chain | NP_000009.1, |
| dehydrogenas | acyl-CoA | NP_001029031.1, |
| (VLCAD) | dehydrogenase | NP_001257376.1, |
| | (VLCAD) | NP_001257377.1 |
| | deficiency | |
| short-chain acyl-CoA | short-chain | NP_000008.1 |
| dehydrogenase | acyl-CoA | |
| (SCAD) | dehydrogenase | |
| | (SCAD) | |
| | deficiency | |
| medium-chain acyl-CoA | medium-chain | NP_000007.1, |
| dehydrogenase (MCAD) | acyl-CoA | NP_001120800.1, |
| | dehydrogenase | NP_001272971.1, |
| | (MCAD) | NP_001272972.1, |
| | deficiency | NP_001272973.1 |
| Myotubularin 1 | X-linked myotubular | NP_000243.1 |
| (MTM1) | myopathy | |
| Myophosphorylase | McArdle disease | NP_001158188.1, |
| (PYGM) | (glycogen | NP_005600.1 |
| | storage disease type V, | |
| | myopho sphory lase | |
| | deficiency) | |
| Lipoprotein | LPL deficiency | NP_000228.1 |
| lipase (LPL) | | |
| sFLT01 (VEGF/P1GF | Age-related macular | SEQ ID NOs: 2, 8,21,2 |
| (placental | degeneration | or 25 of WO |
| growth factor) | | 2009/10566 |
| binding domain of | | |
| human VEGFR1/Flt-1 (hVEGFRl | | |
| fused to the Fc portion of human | | |
| IgG(l) through a polyglycine | | |
| linker) | | |
| Glucocerebrosidase | Gaucher disease | NP_000148.2, |
| (GC) | | NP_001005741.1, |
| | | NP_001005742.1, |
| | | NP_001165282.1, |
| | | NP_001165283.1 |
| UDP g | Crigler-Najjar | NP_000454.1 |
| lucuronosyltransferase 1 | syndrome | |
| family, polypeptide | | |
| A1 (UGT1A1) | | |
| Glucose 6-phosphatase | GSD-Ia | NP_000142.2, |
| (G6Pase) | | NP_001257326.1 |
| Ornithine | OTC deficiency | NP_000522.3 |
| carbamoyltransferase | | |
| (OTC) | | |
| Cystathionine-beta- | Homocystinuria | NP_000062.1, |
| synthase (CBS | | NP_001171479.1, |
| | | NP_001171480.1 |
| Factor VIII (F8) | Hemophilia A | NP_000123.1, |
| | | NP_063916.1 |
| Hemochromatosis | Hemochromatosis | NP_000401.1, |
| (HFE) | | NP_620572.1, |
| | | NP_620573.1, |
| | | NP_620575.1, |
| | | NP_620576.1, |
| | | NP_620577.1, |
| | | NP_620578.1, |
| | | NP_620579.1, |
| | | NP_620580.1 |
| Low density | Phenylketonuria | NP_000518.1, |
| lipoprotein receptor | (PKU) | NP_001182727.1, |
| (LDLR) | | NP_001182728.1, |
| | | NP_001182729.1, |
| | | NP_001182732.1 |
| Galactosidase, alpha (AGA) | Fabry disease | NP_000160.1 |
| Phenylalanine | Hyper- | NP_000268.1 |
| hydroxylase (PAH) | cholesterolaemia | |
| | or | |
| | Phenylketonuria | |
| | (PKU) | |
| Propionyl CoA | Propionic acidaemias | NP_000273.2, |
| carboxylase, alpha | | NP_001121164.1, |
| polypeptide (PCCA) | | NP_001171475.1 |

Other exemplary polypeptides or proteins of interest include clotting factors, globins (e.g., human β-globin and human γ-globin), adrenergic agonists, anti-apoptosis factors, apoptosis inhibitors, cytokine receptors, cytokines, cytotoxins, erythropoietic agents, glutamic acid decarboxylases, glycoproteins, growth factors, growth factor receptors, hormones, hormone receptors, interferons, interleukins, interleukin receptors, kinases, kinase inhibitors, nerve growth factors, netrins, neuroactive peptides, neuroactive peptide receptors, neurogenic factors, neurogenic factor receptors, neuropilins, neurotrophic factors, neurotrophins, neurotrophin receptors, N-methyl-D-aspartate antagonists, plexins, proteases, protease inhibitors, protein decarboxylases, protein kinases, protein kinsase inhibitors, proteolytic proteins, proteolytic protein inhibitors, semaphoring, semaphorin receptors, serotonin transport proteins, serotonin uptake inhibitors, serotonin receptors, serpins, serpin receptors, and tumor suppressors. In some embodiments, the polypeptide or protein of interest is a human protein or polypeptide.

In some embodiments, a transgene encoding the protein or polypeptide of interest is flanked by AAV inverted terminal repeats (ITRs). Thus, in some embodiments, a rAAV nucleic acid vector as described herein comprises ITRs, such as those derived from a wild-type AAV genome. In some embodiments, the ITRs are unmodified, relative to the wild-type AAV ITRs of the same serotype. In some embodiments, the ITRs are modified, relative to the wild-type AAV ITRs of the same serotype. Such modifications may comprise the addition, substitution, or deletion of one or more nucleotides within the ITR nucleic acid sequence. In some embodiments, the AAV ITRs which flank the transgene encoding the protein or polypeptide of interest are or are derived from (e.g., are modified relative to) wild-type AAV1 ITRs. In some embodiments, the AAV ITRs which flank the transgene encoding the protein or polypeptide of interest are or are derived from (e.g., are modified relative to) wild-type AAV2 ITRs. In some embodiments, the AAV ITRs which flank the transgene encoding the protein or polypeptide of interest are or are derived from (e.g., are modified relative to) wild-type AAV3 ITRs. In some embodiments, the AAV ITRs which flank the transgene encoding the protein or polypeptide of interest are or are derived from (e.g., are modified relative to) wild-type AAV4 ITRs. In some embodiments, the AAV ITRs which flank the transgene encoding the protein or polypeptide of interest are or are derived from (e.g., are modified relative to) wild-type AAV5 ITRs. In some embodiments, the AAV ITRs which flank the transgene encoding the protein or polypeptide of interest are or are derived from (e.g., are modified relative to) wild-type AAV6 ITRs.

In some embodiments of the disclosed methods of screening rAAVs for enhanced potency, a nucleic acid encoding an AAV capsid (Cap) protein is flanked by AAV inverted terminal repeats (ITRs). rAAV vectors comprising a Cap-encoding sequence may be comprised within a baculovirus or an rAAV particle, in accordance with the disclosed methods.

Some embodiments of the disclosure contemplate a mammalian cell comprising the nucleic acid as disclosed and/or described herein, and/or a mammalian host cell comprising the rAAV particle as disclosed and/or described herein.

### Methods of Screening

Some embodiments of the present disclosure describe a method of identifying certain Kozak sequences that alter the ratios of VP1, VP2, and VP3 in insect cells by modifying the translation thereof. In some embodiments, the identified Kozak sequences are integrated into a library comprising sequences specific to certain AAV plasmids. In some embodiments, the plasmid library comprises AAV1, AAV2, AAV3, AAV4, AAV5, AAV6, AAV7, AAV8, AAV9, AAV-rh10, AAVrh-74, or AAV-DJ serotypes. In some embodiments, the library comprises plasmids comprising the sequences set forth in Tables 2 or 3. In some embodiments, the library can be screened to identify Kozak sequences of interest. In some embodiments, the screening leads to the identification of certain Kozak sequences that lead to the translation of specific ratios of VP1:VP2:VP3, as those ratios are described herein. In some embodiments, the specific Kozak sequences are those described in Table 2. In some embodiments, the specific Kozak sequences are those described in Table 3. In some embodiments, the specific Kozak sequences are those described in Table 4. In some embodiments, each plasmid library has greater than 100× clonal coverage (e.g., greater than 100× copies of each Kozak variant in the plasmid pool). In some embodiments, one or more Kozak sequences are selected that alter the VP1:VP2:VP3 ratio such that the rAAV produced using the methods described herein in insect (e.g., Sf9) cells display characteristics (e.g., transduction efficiency, infectivity, yield, packing, etc.) that are equivalent to those observed rAAV produced in mammalian (e.g., HEK293T) cells using the same AAV serotype and wild-type Kozak sequence.

In some embodiments, a multiplicity of infection (MOI) of the disclosed methods of screening is contemplated wherein the MOI is about 2, about 3, about 4, about 5, about 10, or about 20. In some embodiments, the MOI of step (a) is about 3. In some embodiments, the multiplicity of infection (MOI) of step (d) is about 1,000.

In some embodiments, the first and/or second insect cell is an Sf9 cell. In some embodiments, the mammalian cell is an HEK293T cell.

Some embodiments contemplate an rAAV particle comprising a capsid protein that is encoded by the nucleic acid as disclosed and/or described herein. In some embodiments, the rAAV is derived from an AAV2 serotype. In some embodiments, the rAAV is derived from an AAV1, AAV2, AAV3, AAV4, AAV5, AAV6, AAV7, AAV8, AAV9, AAV-rh10, AAVrh-74, or AAV-DJ serotype.

Thus, some embodiments contemplate a method of screening for an rAAV particle having enhanced potency or fitness, wherein the rAAV particle is produced in insect cells, wherein one or more Kozak sequences are modified to alter the translation ratio of VP1:VP2:VP3, and wherein the rAAV produced in insect cells display characteristics that are equivalent to an rAAV produced in mammalian cells using the same AAV serotype and the wild-type Kozak sequence.

TABLE 2
First set of pre-tested Kozak
variants (SEQ ID NOs: 1-32)

| SEQ ID NO: | Kozak Sequence |
|---|---|
| 1 | CCGCCCTCATGGC |
| 2 | GGGCCGCAATGGC |
| 3 | CAGTGGCTATGGC |
| 4 | AGTTACTAATGGC |
| 5 | GAAGAGCGATGGC |
| 6 | ACGCCAATATGGC |
| 7 | CTTTGGACATGGC |
| 8 | AGGCAATCATGGC |
| 9 | CGACCGCCATGGC |
| 10 | ACGCCTGTATGGC |
| 11 | ACAAATTCATGGC |
| 12 | TTCCTCTAATGGC |
| 13 | AACACTCAATGGC |
| 14 | AACGATGCATGGC * |
| 15 | CAGAATGGATGGC |
| 16 | CAACATGAATGGC * |
| 17 | GATCATGGATGGC * |
| 18 | GCTTGGAGATGGC |
| 19 | GAACATGTATGGC |
| 20 | GAATATGGATGGC |
| 21 | TAGATTGAATGGC |
| 22 | CGCGATGGATGGC * |
| 23 | CGCAATGGATGGC |
| 24 | GGCAATGGATGGC |
| 25 | TTGATCCAATGGC |
| 26 | CAAAATGGATGGC |
| 27 | CCCGGTGTATGGC |
| 28 | ATACCGACAGGGC |
| 29 | TTCATGACATAGC |
| 30 | GTTTTCTTAGGGC |
| 31 | GTAACGTTATAGC |
| 32 | ACTTTAAAACGGC |

* Four pre-selected variants for that underwent deeper characterization, all for AAV2. Kozak SEQ ID NO: 14 demonstrated almost identical features as AAV2 produced in HEK293 cells.

TABLE 3
Constructed Plasmid libraries
(SEQ ID NOs: 33-42)

| Library | Design | Complexity |
|---|---|---|
| AAV1 | NNNNATGNATGGC (SEQ ID NO: 33) | 4,096 |
| AAV3 | NNNNATGNATGGC (SEQ ID NO: 34) | 4,096 |
| AAV4 | NNNNNNNNATGAC (SEQ ID NO: 35) | 65,536 |
| AAV5 | NNNNNNNNATGTC (SEQ ID NO: 36) | 65,536 |
| AAV6 | NNNNATGNATGGC (SEQ ID NO: 37) | 4,096 |
| AAV7 | NNNNATGNATGGC (SEQ ID NO: 38) | 4,096 |
| AAV8 | NNNNATGNATGGC (SEQ ID NO: 39) | 4,096 |
| AAV9 | NNNNATGNATGGC (SEQ ID NO: 40) | 4,096 |
| AAV-DJ | NNNNATGNATGGC (SEQ ID NO: 41) | 4,096 |
| AAV-rh10 | NNNNATGNATGGC (SEQ ID NO: 42) | 4,096 |

Summary complexity: 163,840

Each plasmid library has >100× clonal coverage (e.g., >100× copies of each Kozak variant in the plasmid pool). The relatively shallow complexity for AAV1, AAV3, AAV6, AAV7, AAV8, AAV9, AAV-DJ, and AAV-rh10 (e.g., 4,096 per serotype) is advantageous, and is a result of the findings generated during the deep sequencing of the AAV2 library. This deep sequencing of the AAV2 library allowed for the identification of certain Kozak sequences of interest, which enabled the efficient search of improved Kozaks for 10 more serotypes using a single high-sequence NGS run.

### Methods of Treatment and Pharmaceutical Compositions

Further provided herein are methods of treating a subject suffering from a disease, disorder or condition, the method comprising administering to the subject any of the disclosed rAAV particles or mammalian cells comprising rAAV particles as disclosed herein. In some embodiments, the subject is a mammal. In some embodiments, the subject is a human, non-human primate, dog, cat, pig, mouse, horse, sheep, goat, rat, guinea pig, hamster, or cow. In some embodiments, the subject is a human.

In some embodiments, “administering” or “administration” means providing a material to a subject in a manner that is pharmacologically useful. In some embodiments, rAAV particles or mammalian cells comprising rAAV particles as described herein are administered to a subject enterally. In some embodiments, an enteral administration of the rAAV particles or mammalian cells comprising rAAV particles as described herein is oral. In some embodiments, rAAV particles or mammalian cells comprising rAAV particles as described herein are administered to the subject parenterally. In some embodiments, rAAV particles or mammalian cells comprising rAAV particles as described herein are administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs. In some embodiments, rAAV particles or mammalian cells comprising rAAV particles as described herein are administered to the subject by injection into the hepatic artery or portal vein. In some embodiments, the rAAV particles or mammalian cells comprising rAAV particles are administered intramuscularly, intravenously, subcutaneously, intrathecally, intraperitoneally, or by direct injection into an organ or a tissue of the subject.

Any one of the AAV particles, capsid proteins, or nucleic acids disclosed herein may be comprised within a pharmaceutical composition comprising a pharmaceutically-acceptable carrier or may be comprised within a pharmaceutically-acceptable carrier. The term “carrier” refers to a diluent, adjuvant, excipient, or vehicle with which the AAV particle, capsid protein, or nucleic acid is comprised or administered to a subject. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers. Non-limiting examples of pharmaceutically acceptable carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, polyacrylic acids, lubricating agents (such as talc, magnesium stearate, and mineral oil), wetting agents, emulsifying agents, suspending agents, preserving agents (such as methyl-, ethyl-, and propyl-hydroxy-benzoates), and pH adjusting agents (such as inorganic and organic acids and bases), and solutions or compositions thereof. Other examples of carriers include phosphate buffered saline, HEPES-buffered saline, and water for injection, any of which may be optionally combined with one or more of calcium chloride dihydrate, disodium phosphate anhydrous, magnesium chloride hexahydrate, potassium chloride, potassium dihydrogen phosphate, sodium chloride, or sucrose. Other examples of carriers that might be used include saline (e.g., sterilized, pyrogen-free saline), saline buffers (e.g., citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of AAV particles to human subjects.

Typically, such compositions may contain at least about 0.1% of the therapeutic agent (e.g., AAV particle) or more, although the percentage of the active ingredient(s) may, of course, be varied and may conveniently be between about 1 or 2% and about 70% or 80% or more of the weight or volume of the total formulation. Naturally, the amount of therapeutic agent(s) (e.g., AAV particle) in each therapeutically-useful composition may be prepared in such a way that a suitable dosage will be obtained in any given unit dose of the compound. Factors such as solubility, bioavailability, biological half-life, route of administration, and product shelf life, as well as other pharmacological considerations, will be contemplated by one skilled in the art of preparing such pharmaceutical formulations, and as such, a variety of dosages and treatment regimens may be designed.

In some embodiments, rAAV particles or mammalian cells comprising rAAV particles as described herein are administered to a subject to treat a disease or condition. To “treat” a disease, as the term is used herein, means to reduce the frequency or severity of at least one sign or symptom of a disease or disorder experienced by a subject. The compositions described above or elsewhere herein are typically administered to a subject in an effective amount, that is, an amount capable of producing a desirable result. The desirable result will depend upon the active agent being administered. For example, an effective amount of rAAV particles may be an amount of the particles that are capable of transferring an expression construct to a host organ, tissue, or cell. A therapeutically acceptable amount may be an amount that is capable of treating a disease, e.g., a disease, disorder, or condition as described in Table 1. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

In embodiments wherein a nucleic acid of the present disclosure is comprised in a composition comprising an AAV particle, the concentration of AAV particles administered to a subject may be on the order ranging from 10⁶ to 10¹⁴ particles/ml or 10³ to 10¹⁵ particles/ml, or any values therebetween for either range, such as for example, about 10⁶, 10⁷, 10⁸, 10⁹, 10¹⁰, 10¹¹, 10¹², 10¹³, or 10¹⁴ particles/ml. In some embodiments, AAV particles of a higher concentration than 10¹³ particles/ml are administered. In some embodiments, the concentration of AAV particles administered to a subject may be on the order ranging from 10⁶ to 10¹⁴ vector genomes (vgs)/ml or 10³ to 10¹⁵ vgs/ml, or any values therebetween for either range (e.g., 10⁶, 10⁷, 10⁸, 10⁹, 10¹⁰, 10¹¹, 10¹², 10¹³, or 10¹⁴ vgs/ml). In some embodiments, AAV particles of higher concentration than 10¹³ vgs/ml are administered. The AAV particles can be administered as a single dose, or divided into two or more administrations as may be required to achieve therapy of the particular disease or disorder being treated. In some embodiments, 0.0001 ml to 10 ml are delivered to a subject. In some embodiments, the number of AAV particles administered to a subject may be on the order ranging from 10⁶-10¹⁴ vgs/kg body mass of the subject, or any values therebetween (e.g., 10⁶, 10⁷, 10⁸, 10⁹, 10¹⁰, 10¹¹, 10¹², 10¹³, or 10¹⁴ vgs/kg). In some embodiments, the dose of AAV particles administered to a subject may be on the order ranging from 10¹²-10¹⁴ vgs/kg. In some embodiments, the volume of a composition comprising an AAV particle delivered to a subject (e.g., via one or more routes of administration as described herein) is 0.0001 ml to 10 ml.

In some embodiments, a composition disclosed herein (e.g., comprising an rAAV particle or nucleic acid of the disclosure) is administered to a subject once. In some embodiments, the composition is administered to a subject multiple times (e.g., twice, three times, four times, five times, six times, or more). Repeated administration to a subject may be conducted at a regular interval (e.g., daily, every other day, twice per week, weekly, twice per month, monthly, every six months, once per year, or less or more frequently) as necessary to treat (e.g., improve or alleviate) one or more symptoms of a disease, disorder, or condition in the subject.

In some embodiments, one or more cells isolated from a subject are contacted with an rAAV particle of the disclosure. In some embodiments, these cells are subsequently administered to a subject, e.g. the same subject from which the cell was isolated.

In some embodiments, the subject has or is suspected of having a disease or disorder that may be treated with gene therapy. In some embodiments, a nucleic acid isolated or derived from the subject (e.g., genomic DNA, mRNA, or cDNA from the subject) is identified via sequencing (e.g., Sanger or next-generation sequencing) to comprise a mutation (e.g., in a gene associated with muscle development, health, maintenance, or function). In some embodiments, the disease or disorder is selected from Table 1.

In some embodiments, the method comprises contacting a cell with an rAAV particle or nucleic acid as described herein. In some embodiments, a cell disclosed herein is a cell isolated or derived from a subject. In some embodiments, a cell is a mammalian cell (e.g., a cell isolated or derived from a mammal). In some embodiments, the cell is a human cell, non-human primate cell, rat cell, or mouse cell. In some embodiments, a cell is isolated or derived from a particular tissue of a subject, such as liver tissue. In some embodiments, the cell is a liver, brain, heart or retina cell. In some embodiments, a cell is a liver cell. In some embodiments, a cell is in vitro. In some embodiments, a cell is ex vivo. In some embodiments, a cell in in vivo. In some embodiments, a cell is within a subject (e.g., within a tissue or organ of a subject). In some embodiments, a cell is a primary cell. In some embodiments, a cell is from a cell line (e.g., an immortalized cell line). In some embodiments a cell is a cancer cell or an immortalized cell.

Methods of contacting a cell may comprise, for example, contacting a cell in a culture with a nucleic acid or rAAV particle as described herein. In some embodiments, contacting a cell comprises adding a nucleic acid or an rAAV particle as described herein to the supernatant of a cell culture (e.g., a cell culture on a tissue culture plate or dish) or mixing a nucleic acid or rAAV particle as described herein with a cell culture (e.g., a suspension cell culture). In some embodiments, contacting a cell comprises mixing a nucleic acid or rAAV particle as described herein with another solution, such as a cell culture media, and incubating a cell with the mixture.

In some embodiments, contacting a cell with a nucleic acid or rAAV particle as described herein comprises administering nucleic acid or rAAV particle as described herein to a subject or device in which the cell is located. In some embodiments, contacting a cell comprises injecting nucleic acid or rAAV particle as described herein into a subject in which the cell is located. In some embodiments, contacting a cell comprises administering nucleic acid or rAAV particle described herein directly to a cell, or into or substantially adjacent to a tissue of a subject in which the cell is present.

### EXAMPLES

### Example 1

The widespread successful use of recombinant Adeno-associated virus (rAAV) in gene therapy has driven the demand for scale-up manufacturing methods of vectors with improved yield and transduction efficiency. The Baculovirus/Sf9 system is a promising platform for high yield production; however, a major drawback to using an invertebrate cell line compared to a mammalian system is a generally altered AAV capsid stoichiometry resulting in lower biological potency.

Hereby, a term of a structural and biological “fitness” of an AAV capsid is introduced as a function of two interdependent parameters—(1) packaging efficiency (yield), and (2) transduction efficiency (infectivity). Both of these parameters are critically dependent on AAV capsid structural proteins VP1/2/3 stoichiometry. To identify an improved AAV capsid composition, a novel Directed Evolution (DE) protocol for assessing structural and biological fitness of Sf9-manufactured rAAV for any given serotype has been developed.

The approach involves the packaging of a combinatorial capsid library in insect Sf9 cells, followed by a library screening for high infectivity in human Cre-recombinase-expressing C12 cells, a mouse cell line. One single DE selection round, complemented by Next-Generation Sequencing (NGS) and guided by in silico analysis, identifies a small subset of VP1 translation initiation sites (TIS; also known as Kozak sequence) encoding “fit” AAV capsids characterized by high production yield and superior transduction efficiencies.

The protocol includes the several features, including a newly designed AAV TR cassette plasmid vector incorporating (1) a combinatorial capsid gene library expressed in Sf9 cells; (2) GFP reporter gene; and (3) Cre-recombinase-controlled flip Lox-sites activated in C12 cells. Reference is made to the Cre-recombinase-restricted selection protocol described in International Patent Publication No. WO 2019/0158619, published Aug. 22, 2019, which is herein incorporated by reference. Additionally, the complexity of an AAV capsid library of ˜10e5 TIS permutations allows for the assembly of rAAV capsids with dynamic VP1:VP2:VP3 stoichiometry. It is important to note that combinatorial libraries are subjected to orthogonal selection pressures. For example, Sf9 insect cells favor the most productive capsid ratios for greater yield (e.g., with a higher VP3 content), while mammalian cells preferentially select for more infectious AAV (e.g., with a higher VP1 content). In the protocol disclosed herein, NGS analysis and unique double barcoding combinations allowed for sequencing of all the theoretical capsid gene permutations with ˜100× coverage overlap. Finally, improved “fit” capsid composition providing for both good yield and infectivity is identified by a machine-guided workflow using several consecutive filtering steps.

As a proof of principle, a combinatorial plasmid library of AAV2 capsid gene variants incorporating all 65,536 possible permutations in the VP1 Kozak sequence for standard ATG initiation (NNNNNNNNATGGC) (SEQ ID NO: 48) and ˜15,000 permutations incorporating non-canonical initiation codons was generated. Using the described workflow, an improved VP1 TIS sequence generating VP1:VP2:VP3 with favorable stoichiometry and biological potency was identified. Using similar algorithm, these findings were extended to other AAV serotypes manufactured in insect Sf9 cells.

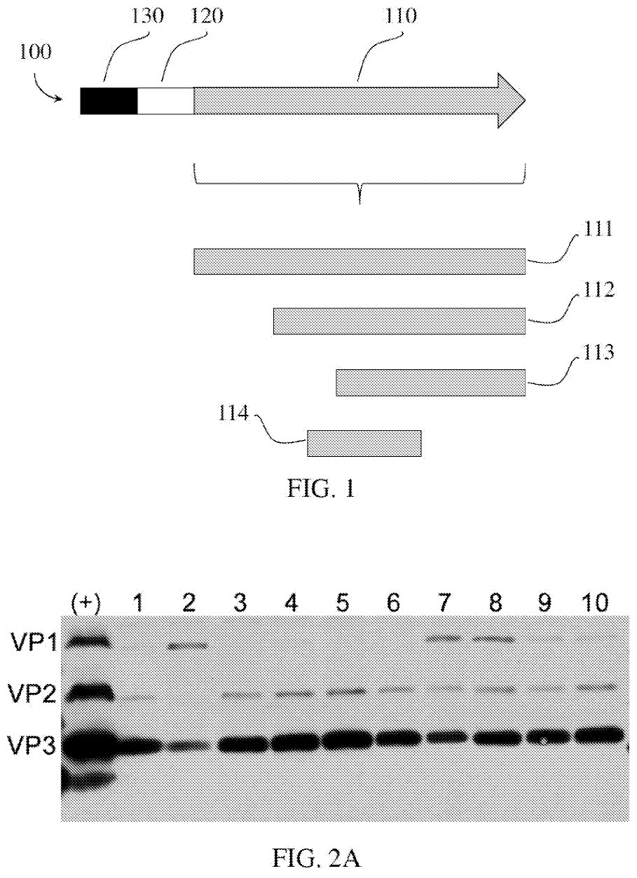

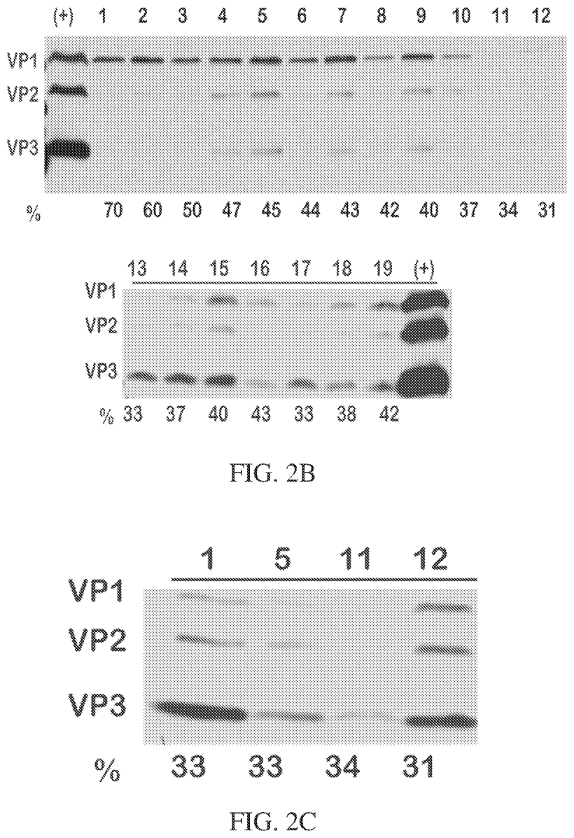

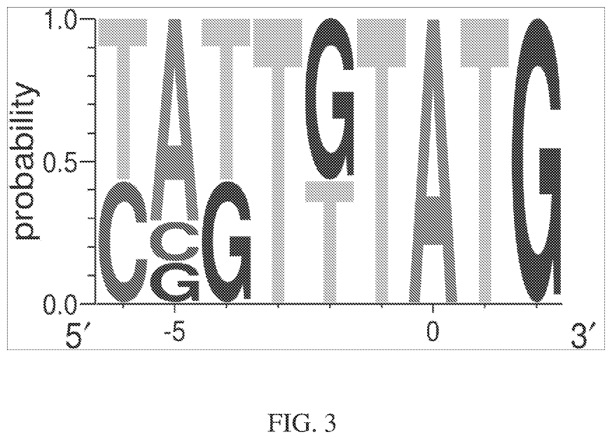

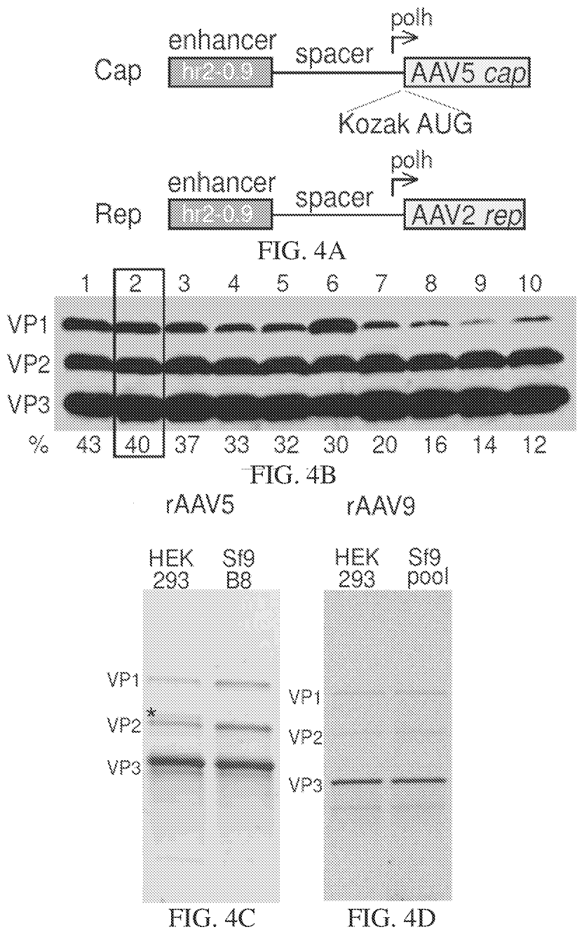

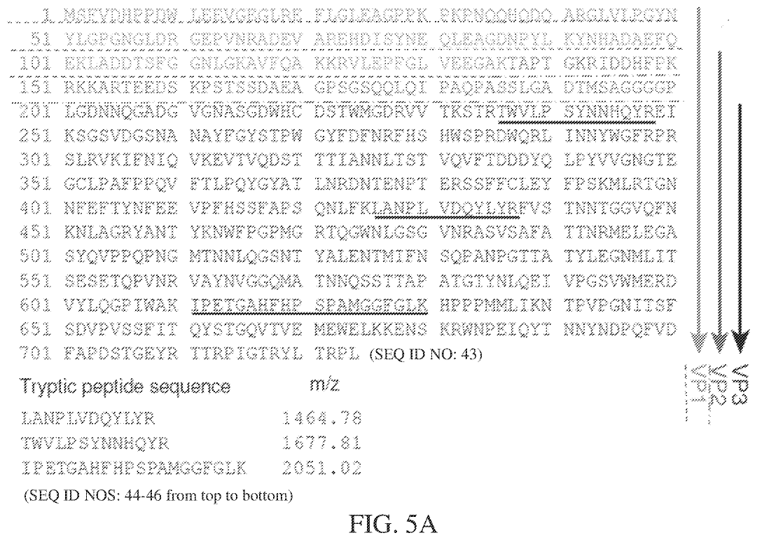

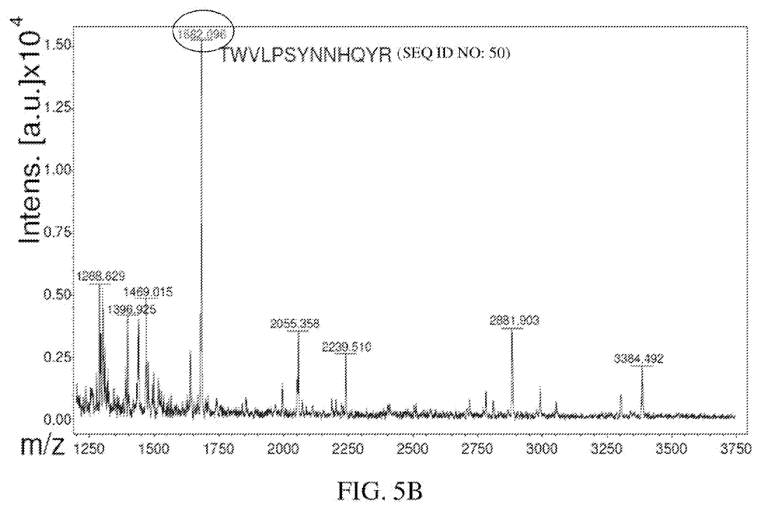

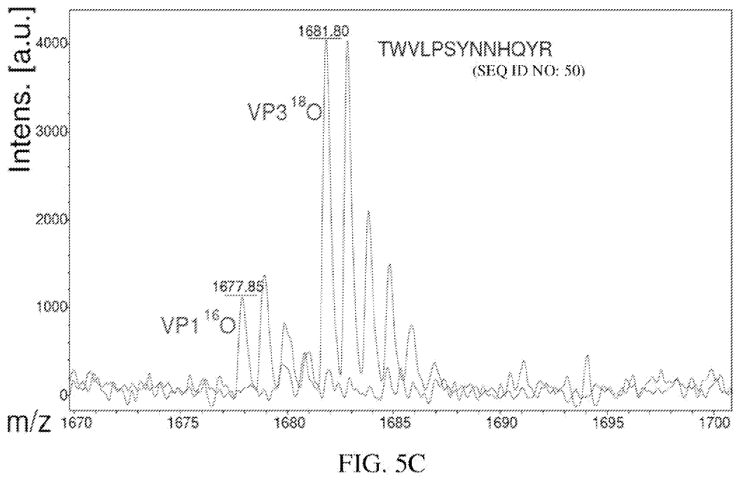

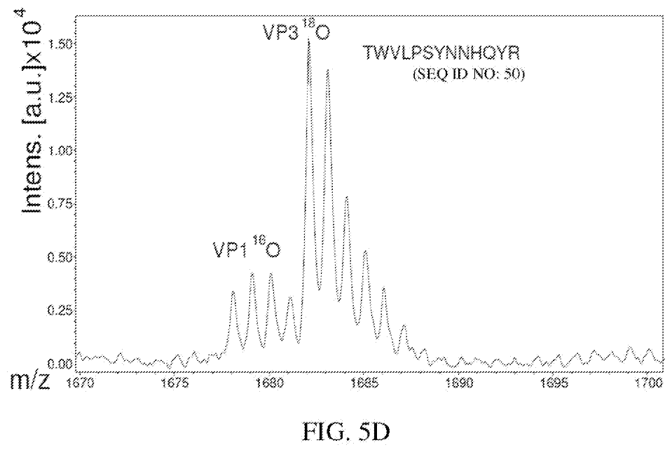

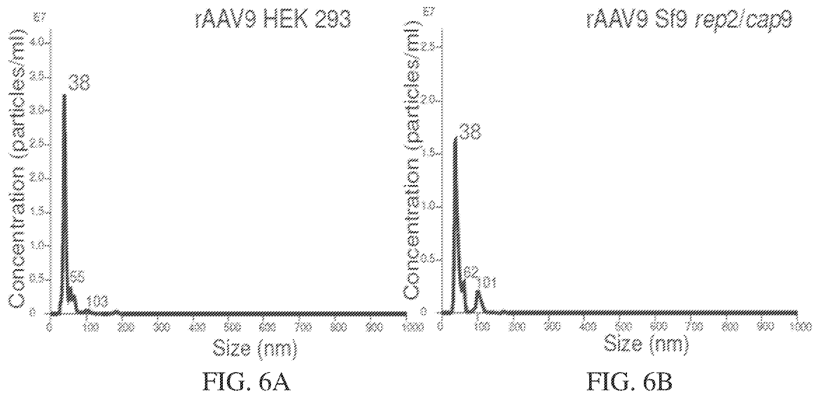

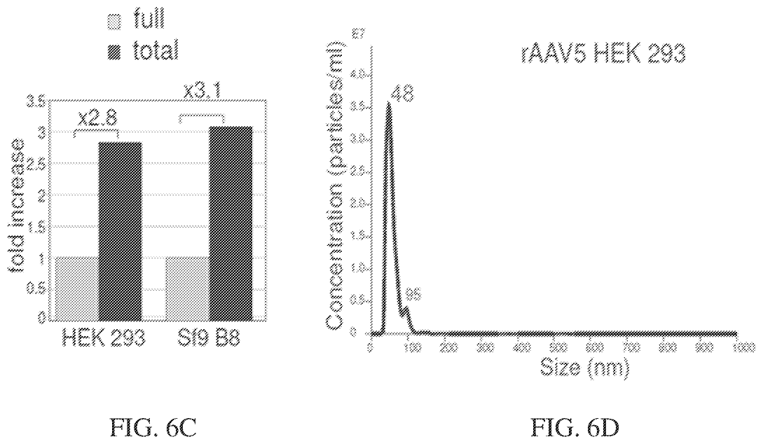

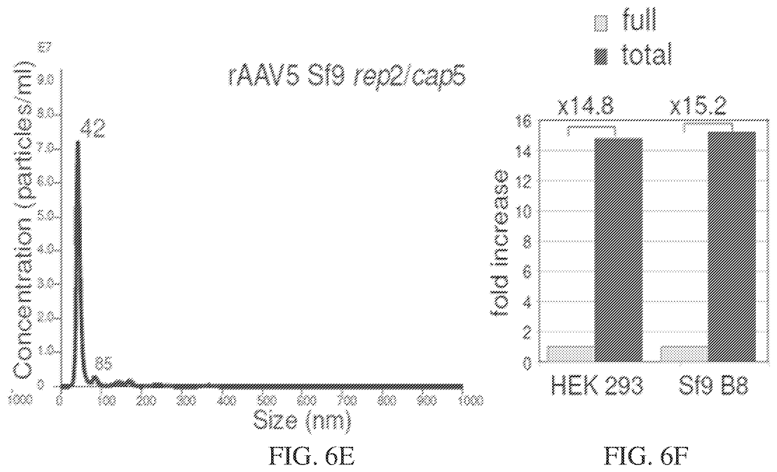

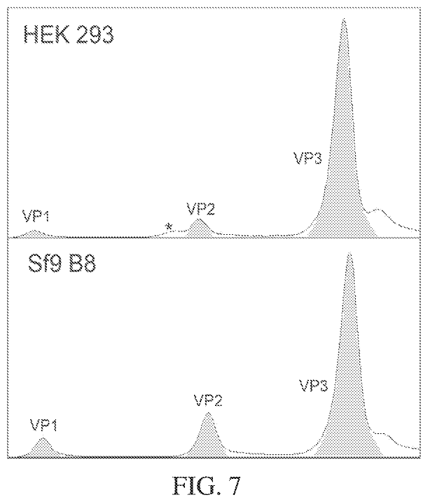

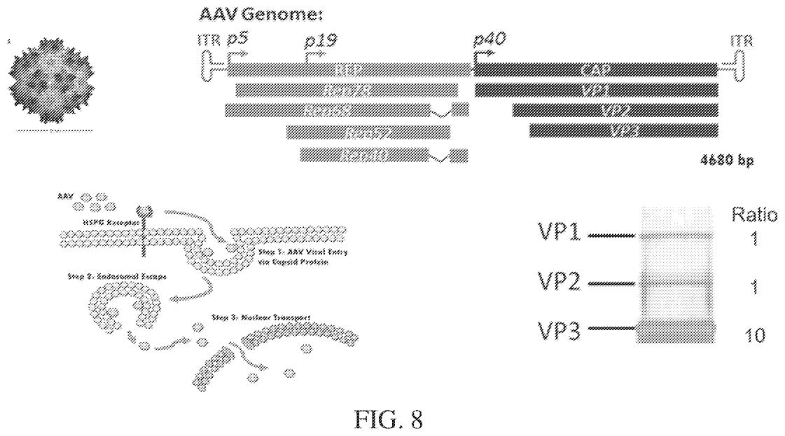

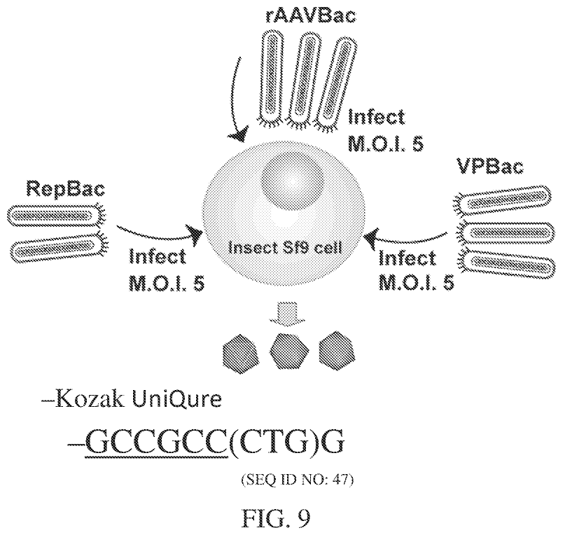

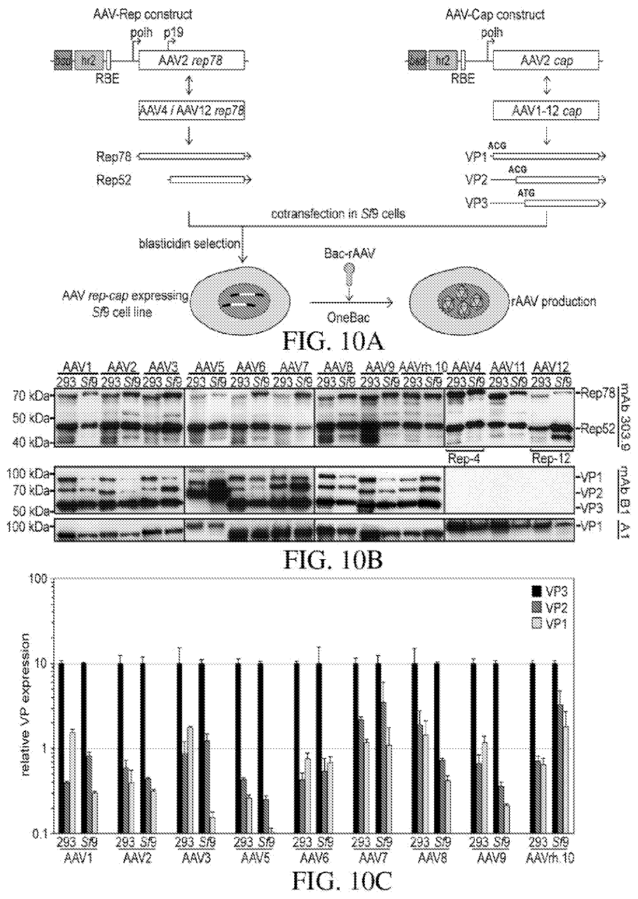

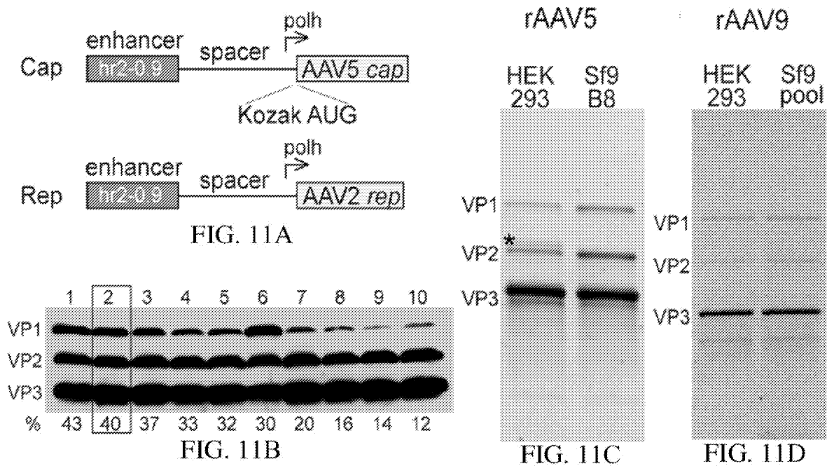

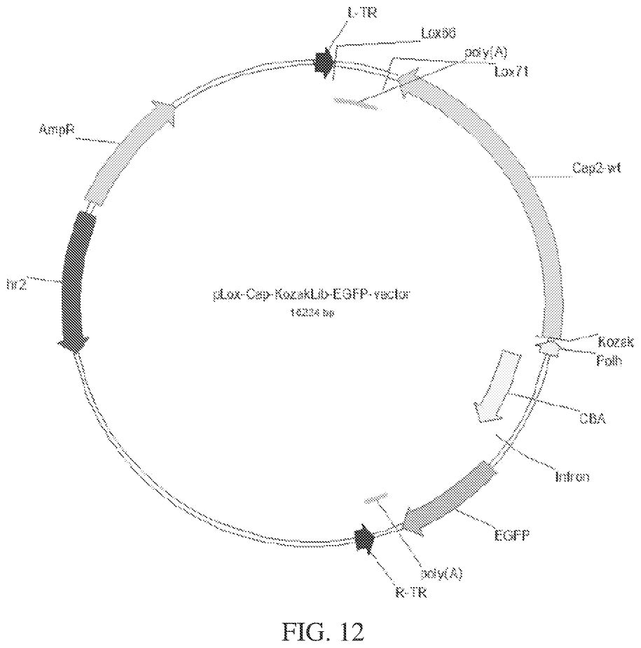

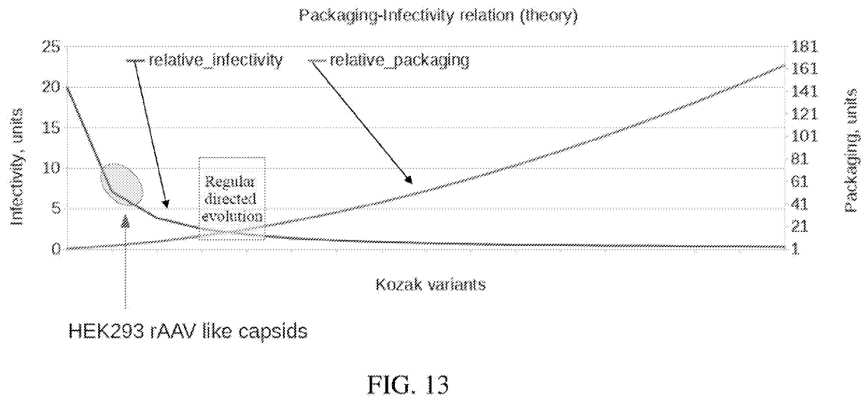

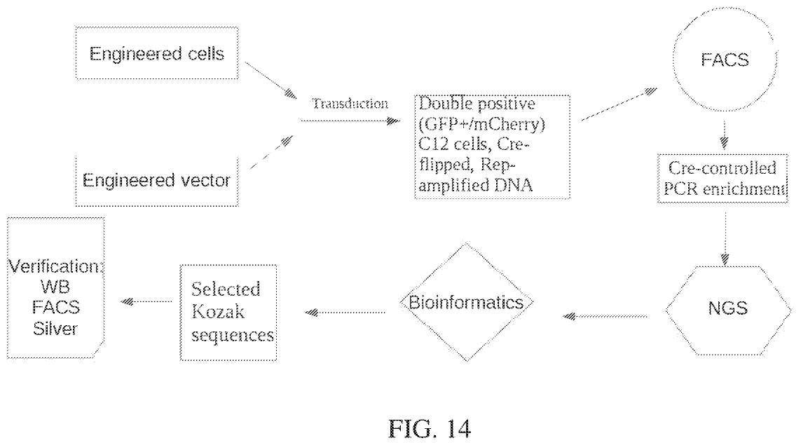

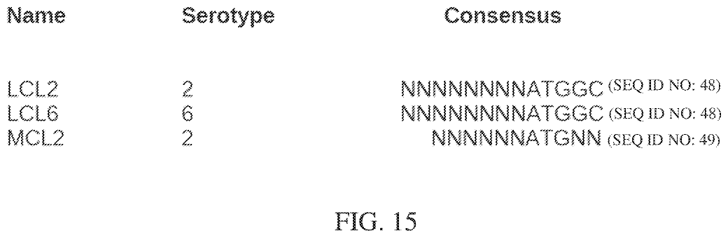

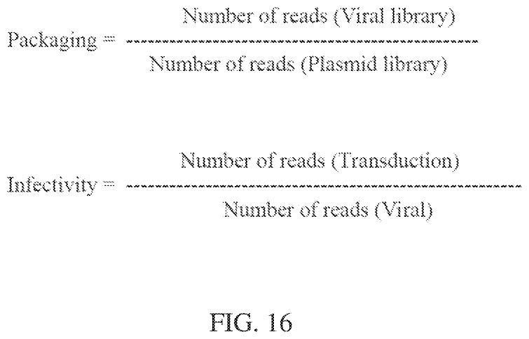

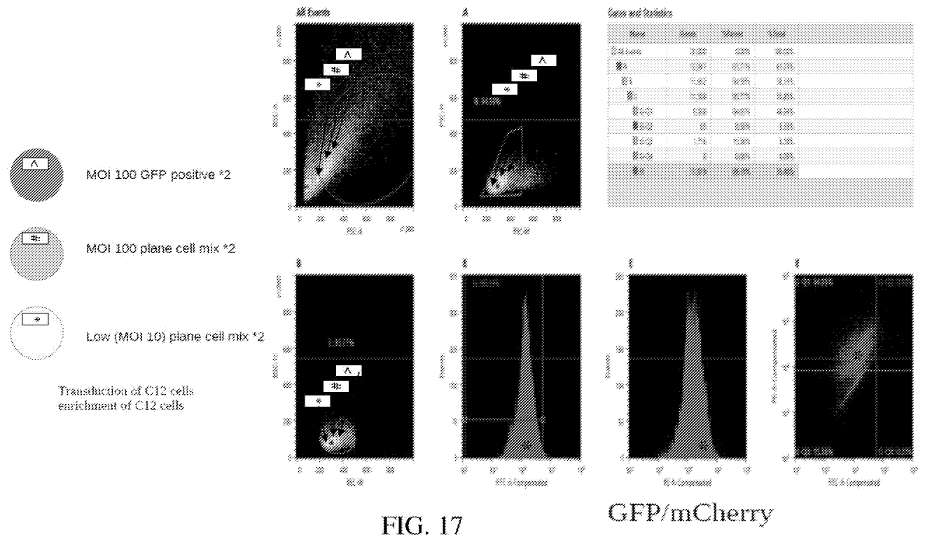

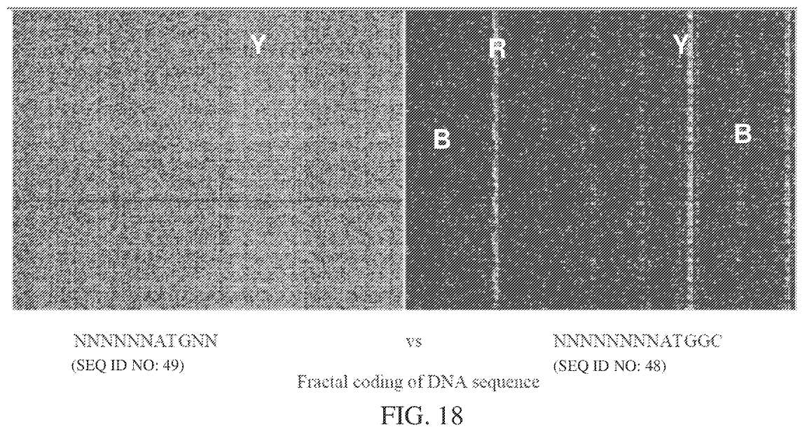

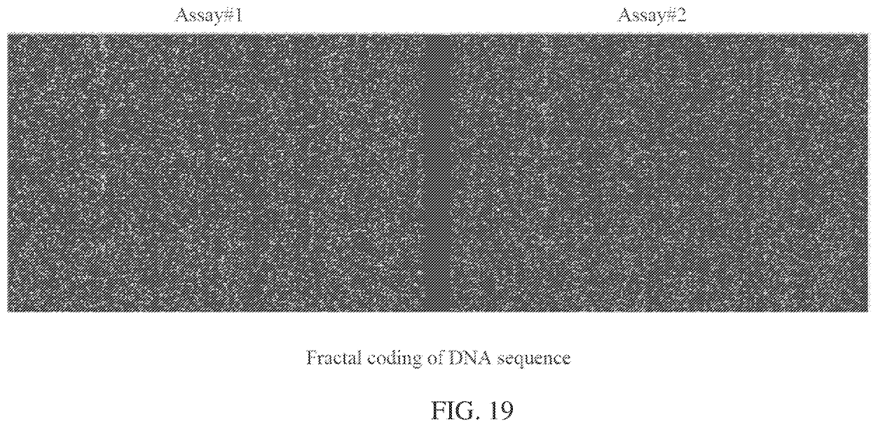

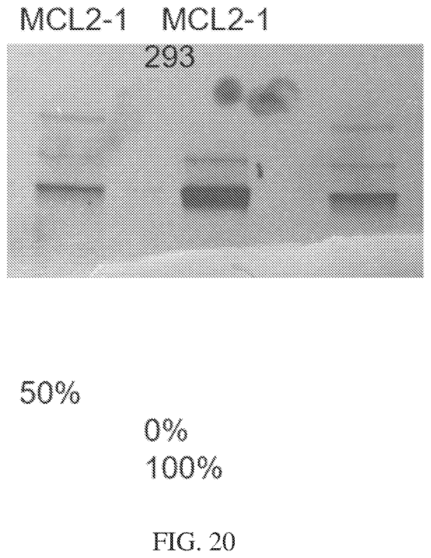

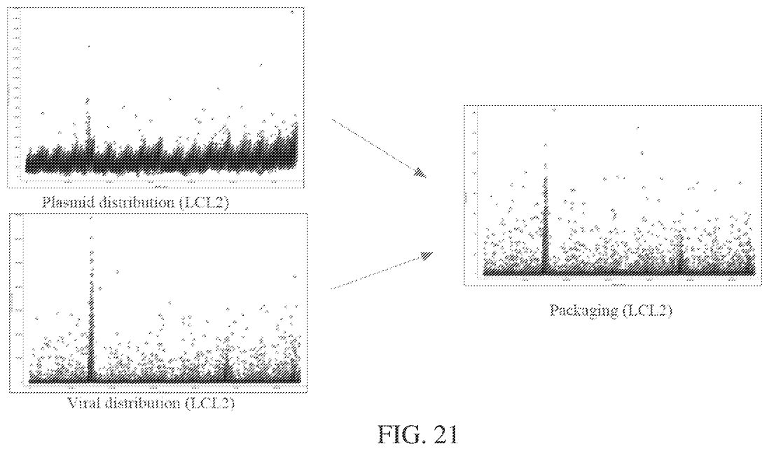

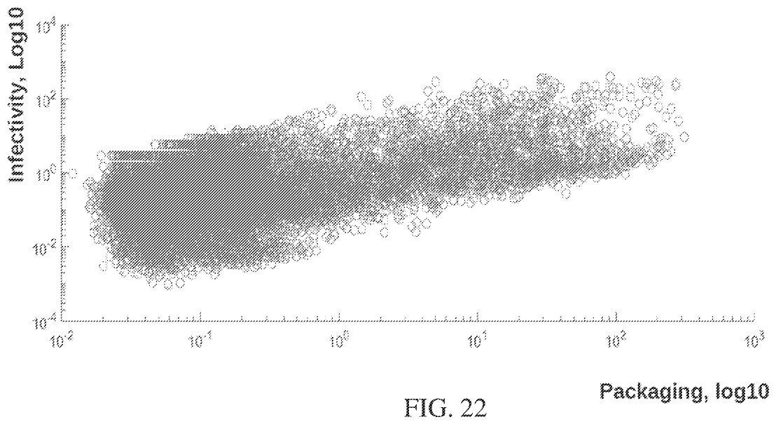

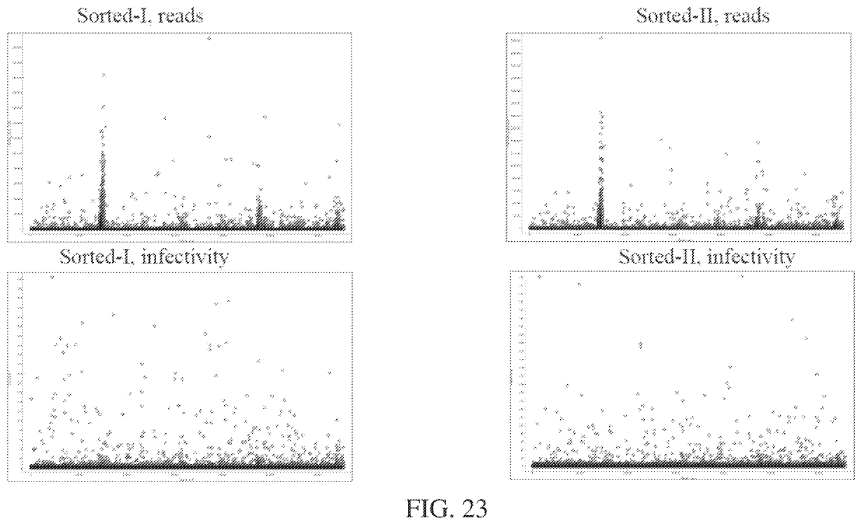

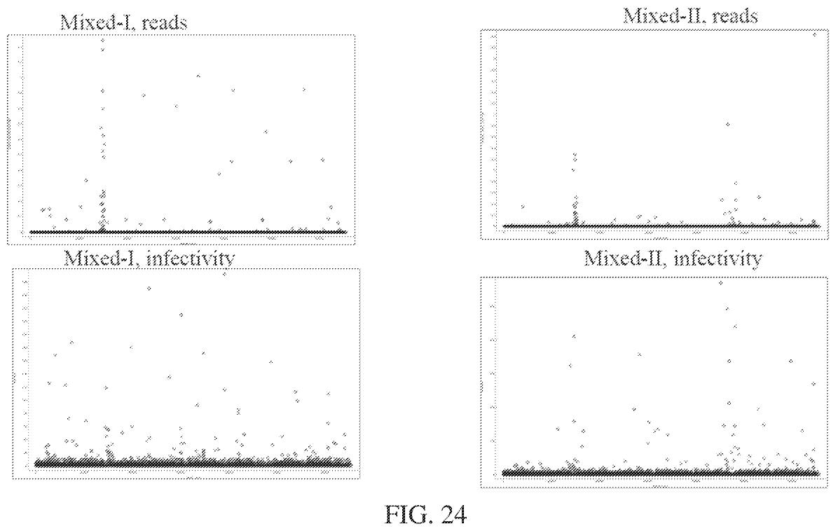

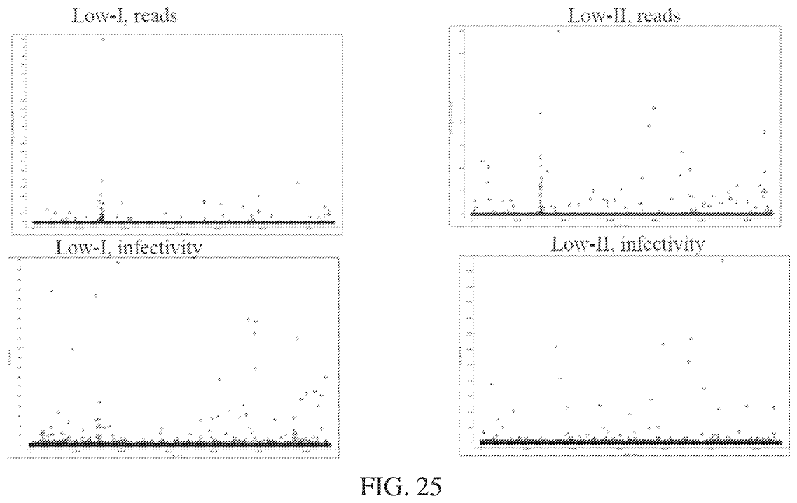

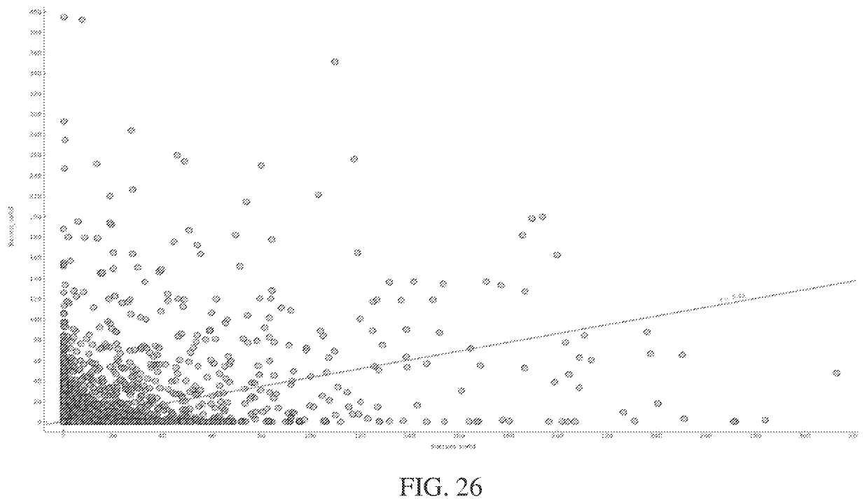

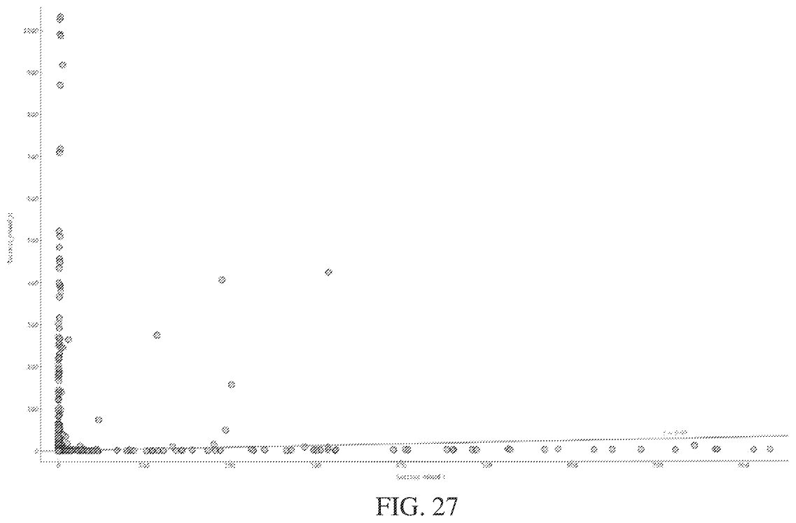

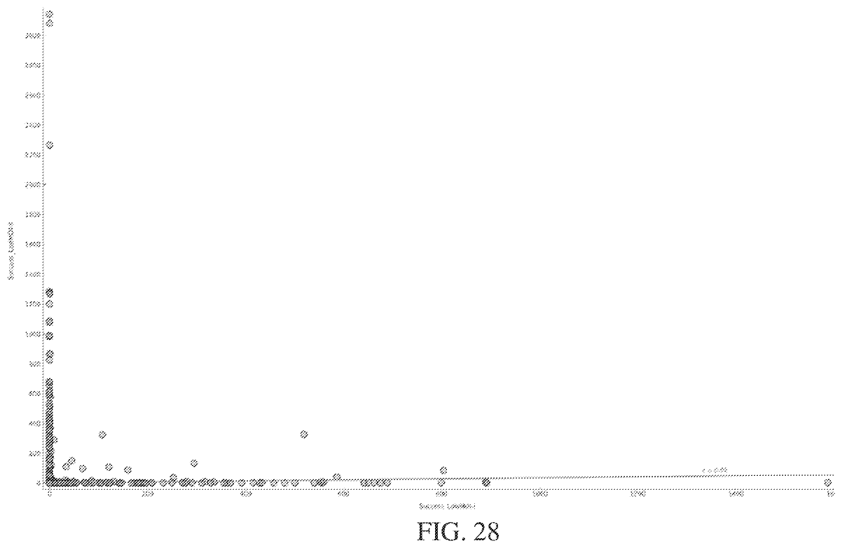

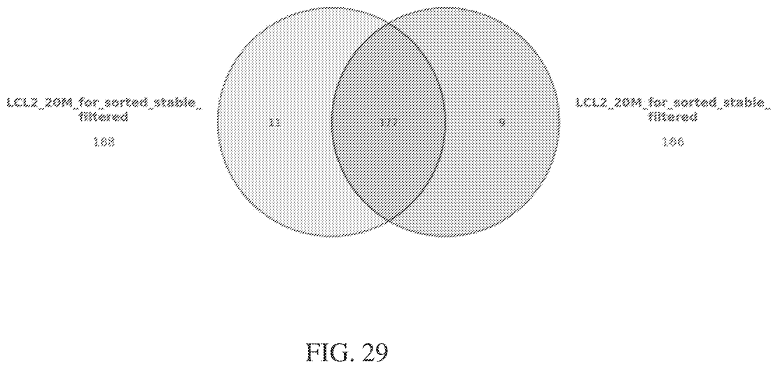

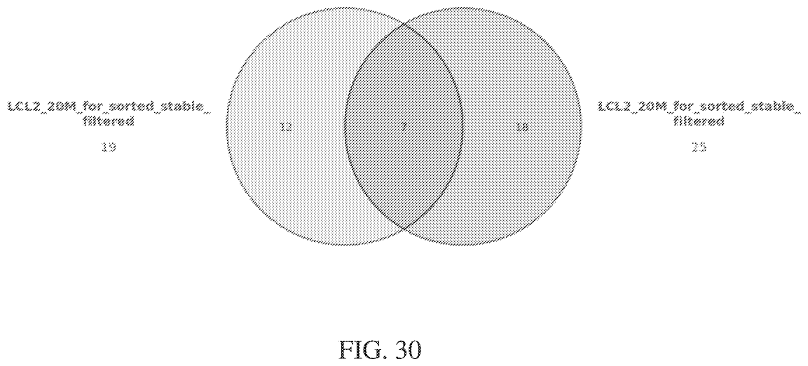

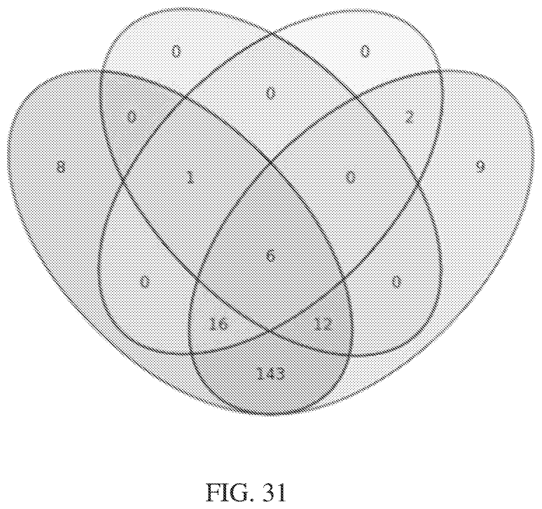

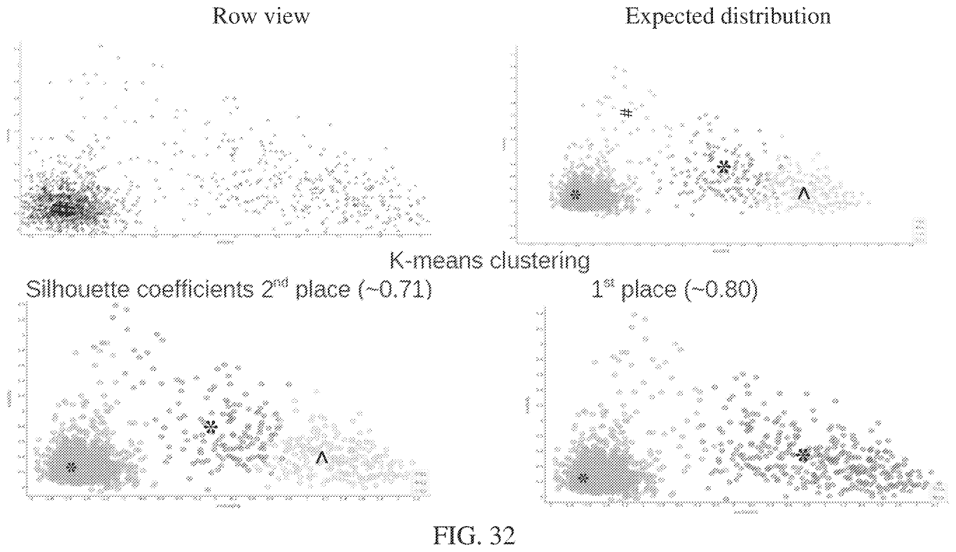

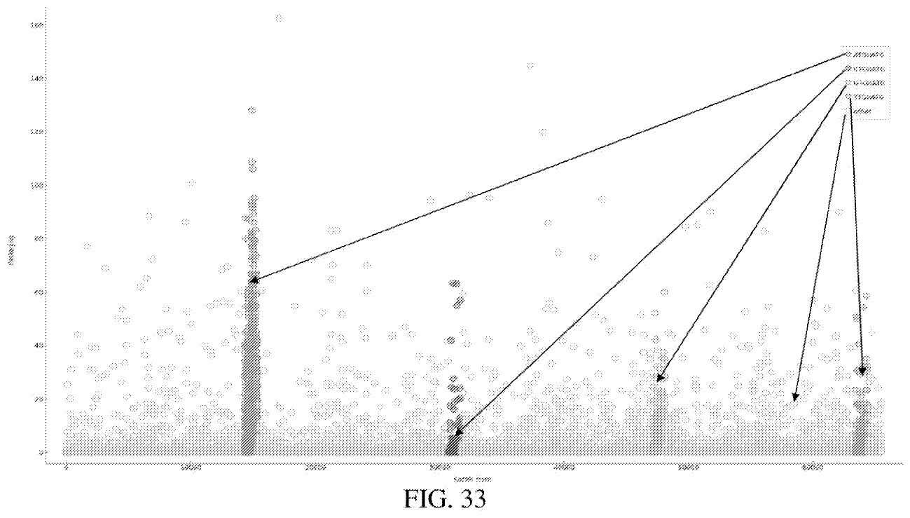

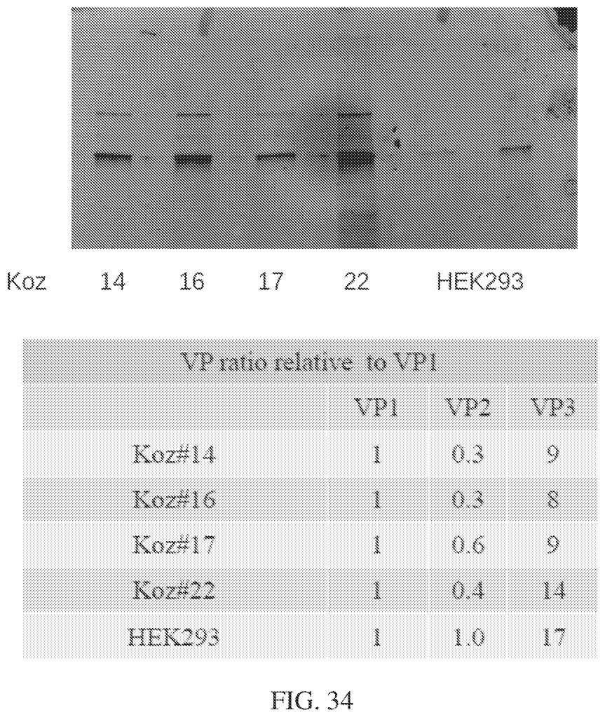

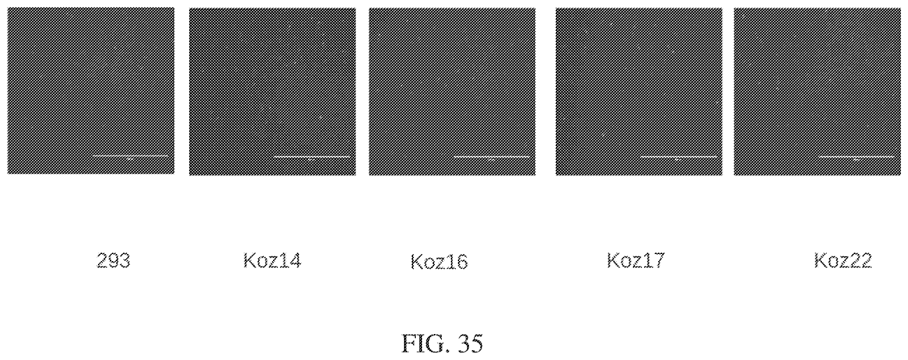

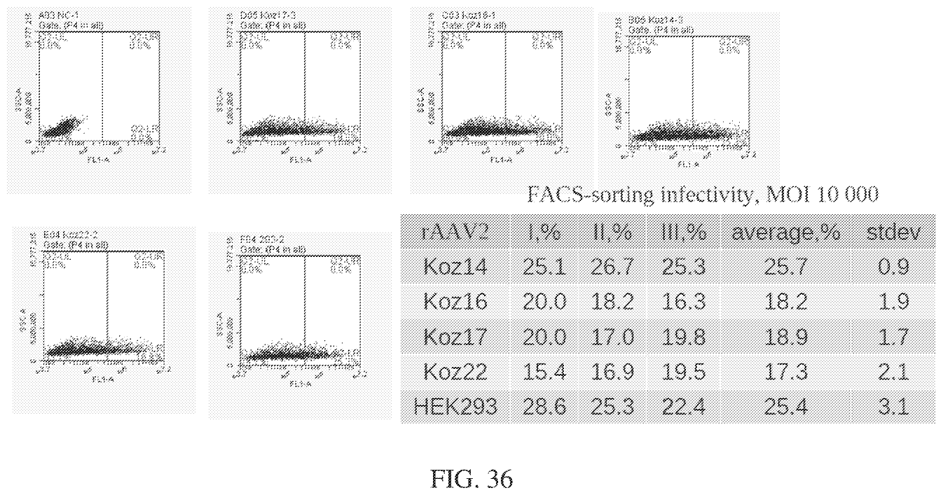

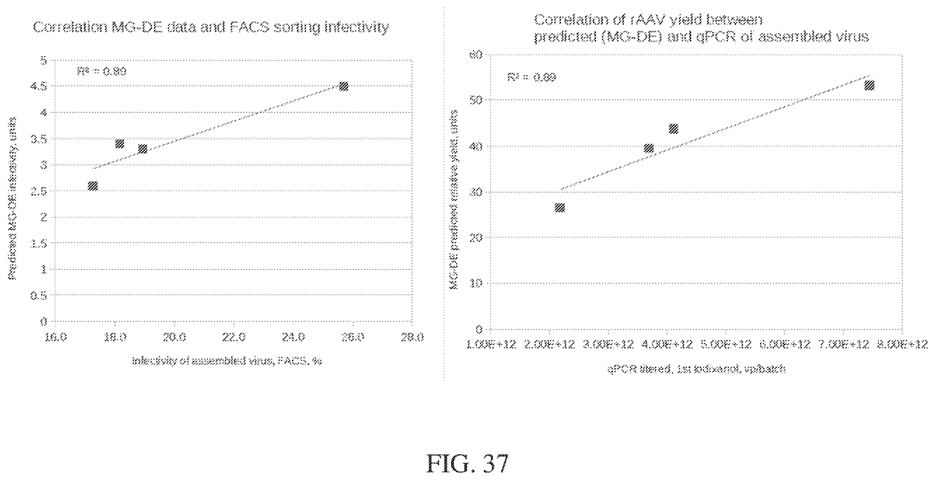

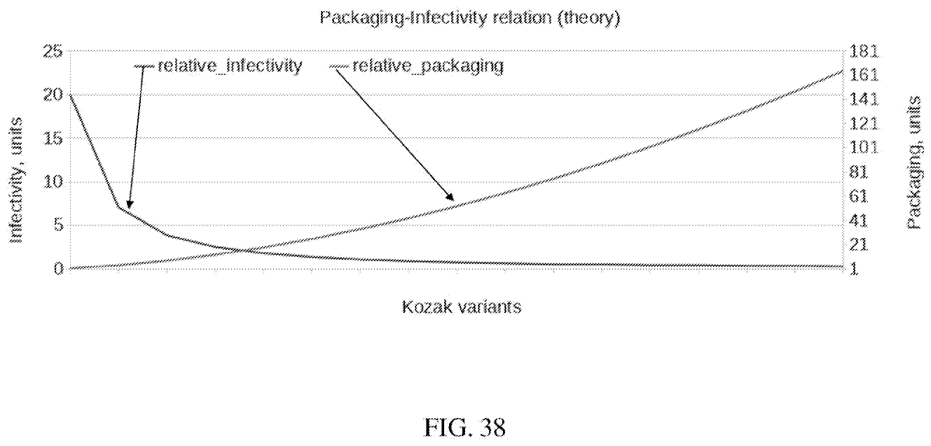

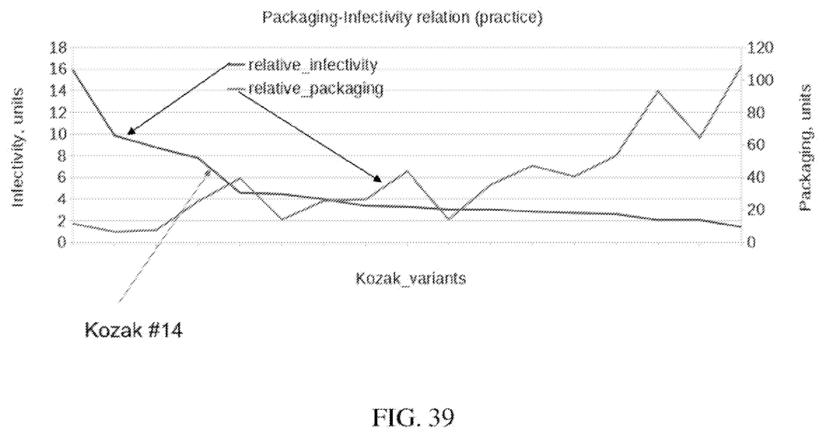

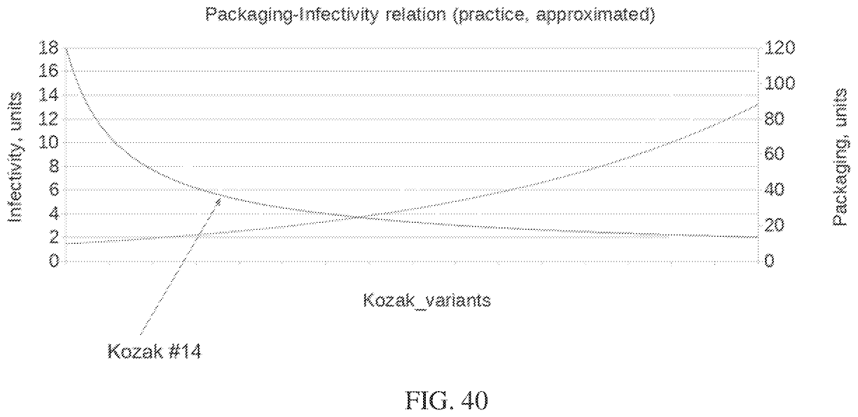

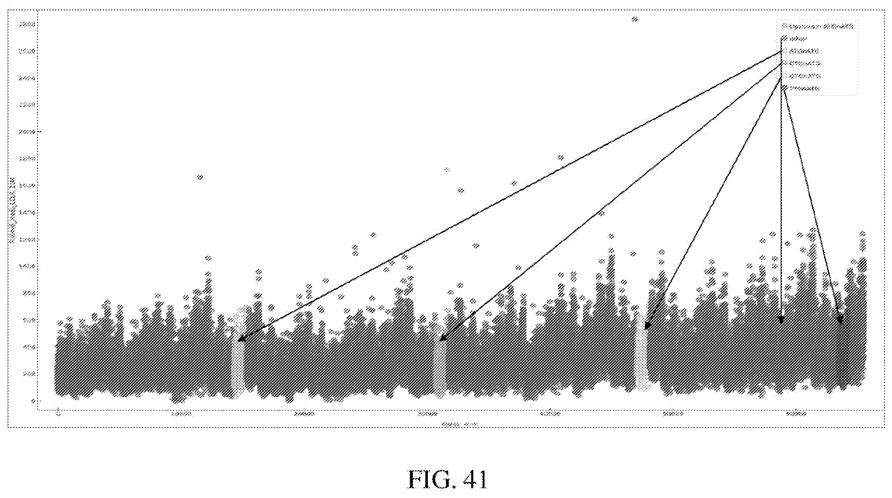

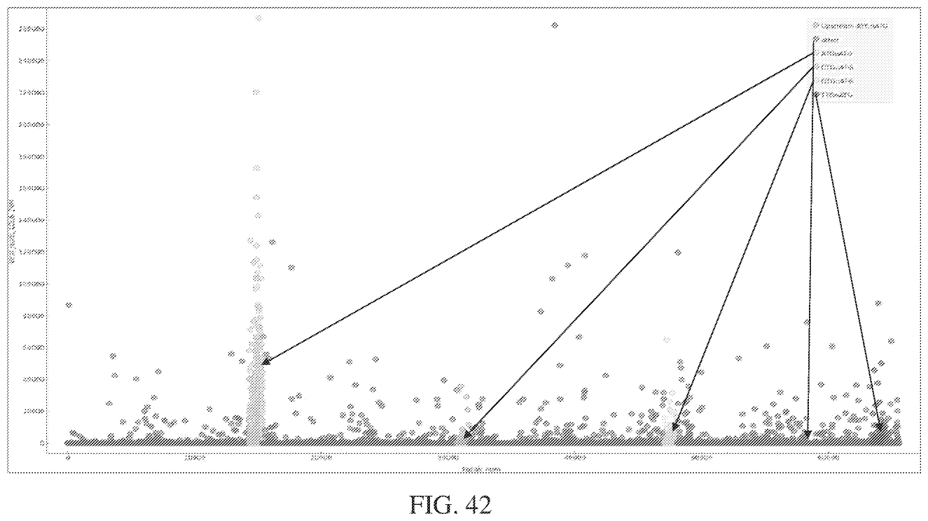

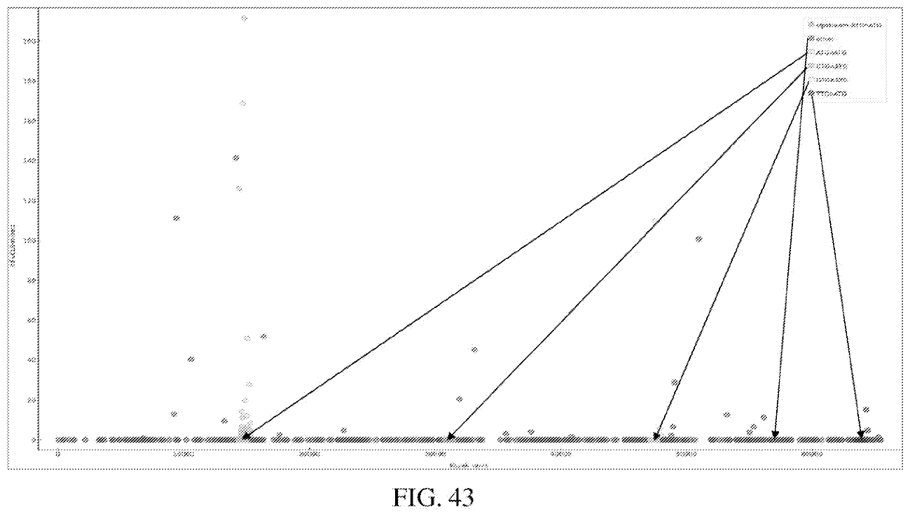

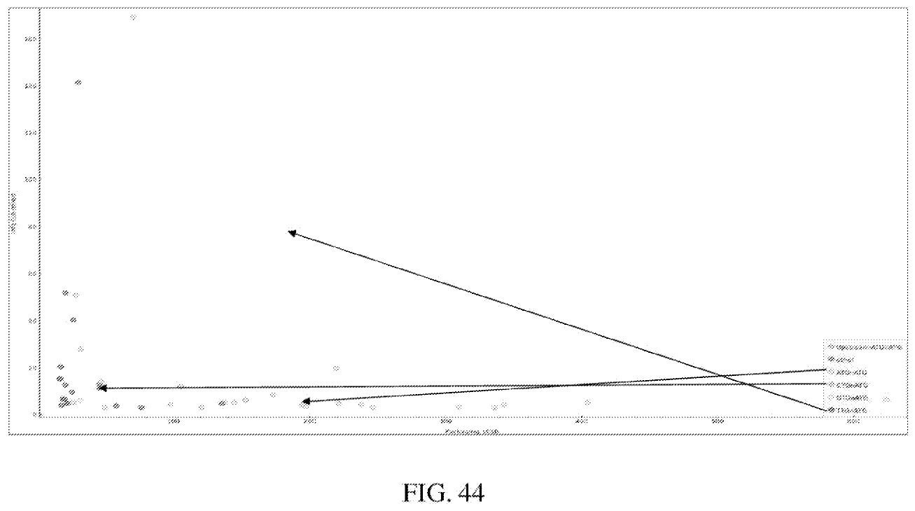

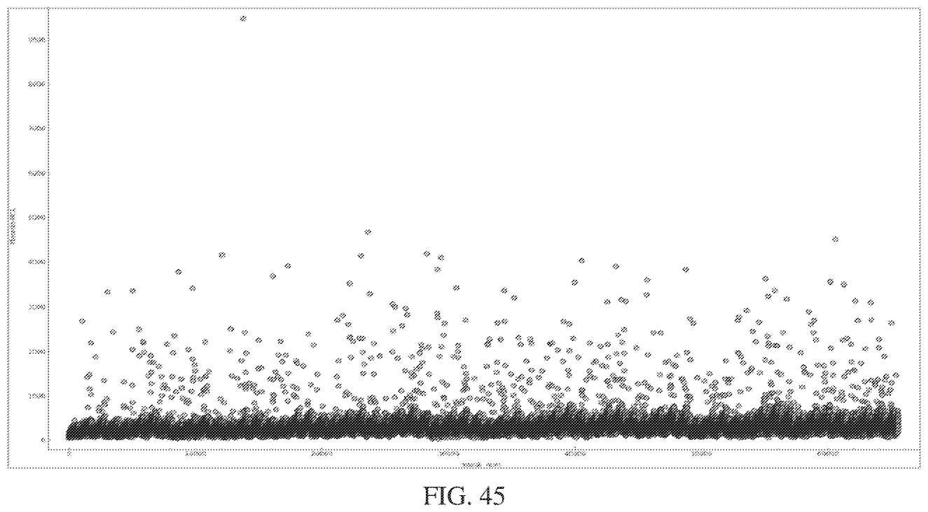

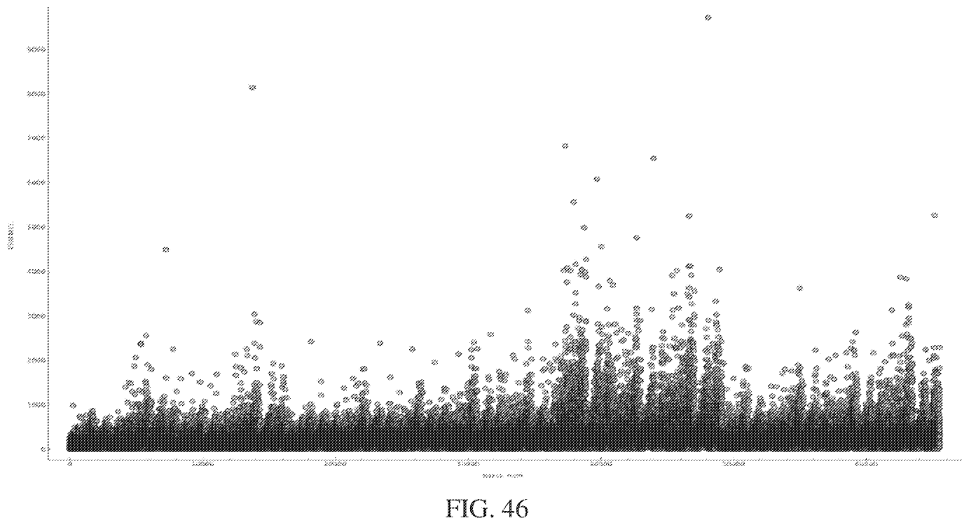

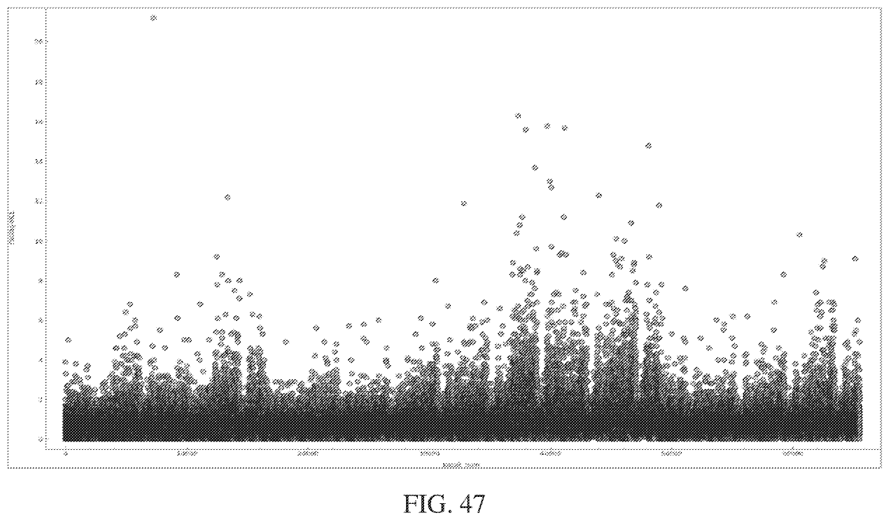

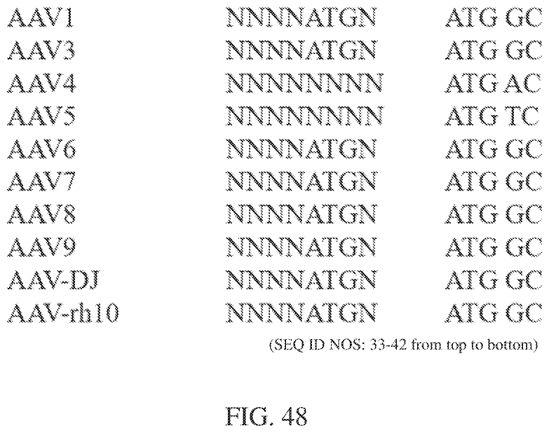

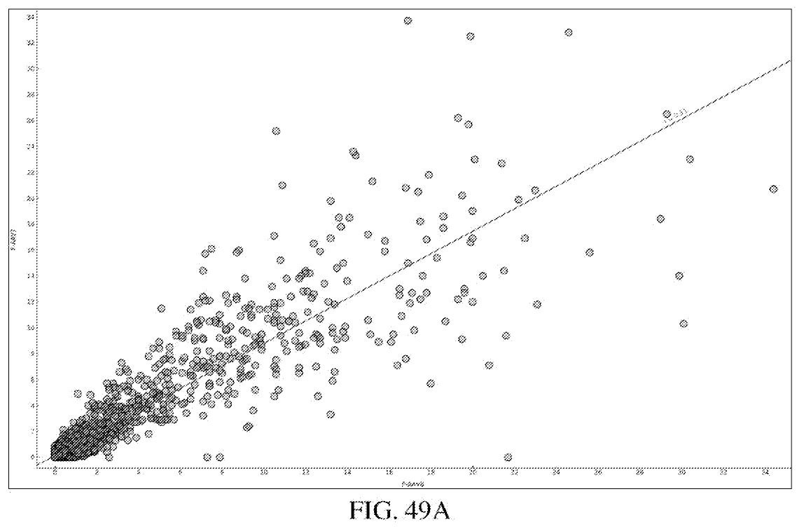

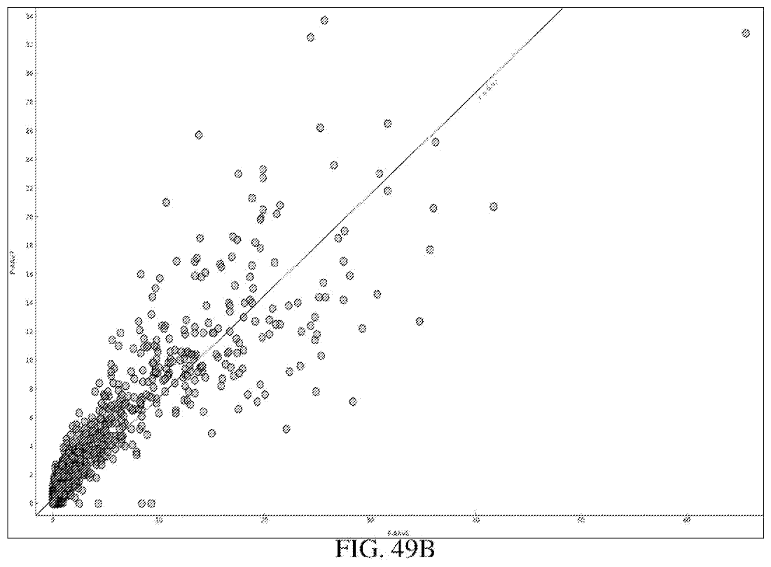

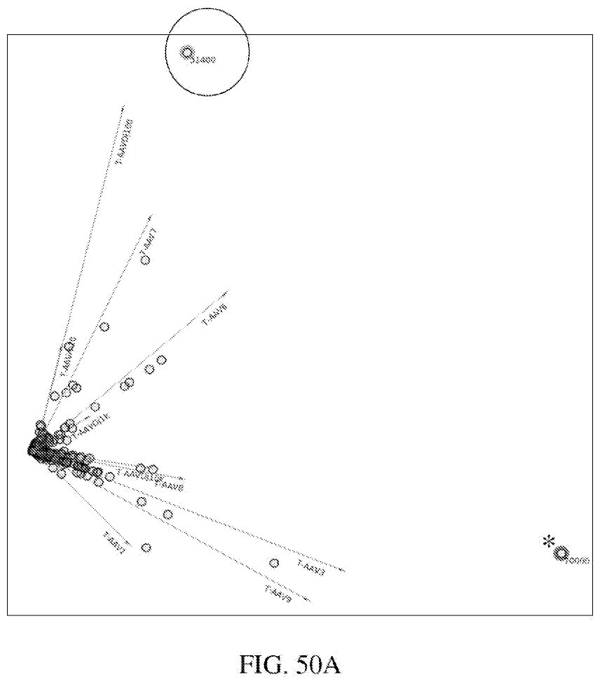

FIGS. 1-48 illustrate the development and empirical testing of the combinatorial plasmid library described in Example 1, from which certain modified Kozak sequences were identified according to the methods described herein.

### Example 2

In order to identify additional sets of Kozak sequences for production AAV with improved yield and VP1 content that is crucial for AAV infectivity (endosomal escape stage), the Directed Evolution (DE) approach described herein was combined with several functional filters linked to the biological fate of AAV capsids within the transduction process.

A Kozak sequence was linked to the AAV capsid inside an ITR-cassette to preserve the genotype-phenotype linkage inside of each AAV particle. AAV capsid sequence was linked to Lox sequences (head-to head orientation). After successful delivery of the AAV cassette, in the presence of Cre recombinase, the delivered DNA undergoes recombination (e.g., a flip of Lox sequences) that generates distinctive sequence. Such distinctive sequence can be selectively amplified by PCR. The cassette carries GFP protein for downstream selection of transduced cells.

A stable cell line based on C12 cells that expresses Cre recombinase was created under Tet/Dox-regulatory element and was termed C12Cre. The C12Cre cell line carries a copy of the Rep gene that can be activated in the presence of helper virus Adenovirus-5 (Ad5). Additionally, the C12Cre cell line expresses mCherry protein.

After a transduction of C12Cre cells by AAV-Kozak libraries, Cre-recombinase expression was stimulated by Dox/Tet, and second strand DNA synthesis and Rep expression was activated using Ad5.

The following four factors play a crucial role in the selection process: (i) the second strand DNA synthesis prepares template for Cre-recombinase; (ii) Rep expression leads to rescue and burst of dsDNA copies in the transduced cell; (iii) Cre-recombinase generates novel flipped sequences for PCR selection; and (iv) increased amount of dsDNA (coding Cap linked to GFP) leads to visible fluorescence of transduced cells, which is used for FACS-based enrichment of positive cells.

To verify the functionality of the approach, a Kozak variant library was generated for AAV2 with subsequent design NNNN NNNN ATG GC (SEQ ID NO: 48) and a complexity of 48=65,536 variants. The library was packaged in Sf9 cells at low plasmid copy per cell conditions. For C12Cre cell transduction, moderate multiplicity of infection was selected, which provides an adequate amount of GFP positive cells for FACS enrichment and has only moderate potential for a co-transduction effect. For example, when a cell is transduced by several capsids, only one functionally active capsid (driver) can rescue non-functional capsids (passangers) during endosomal escape. Forty-eight hours following the addition of the AAV-Kozak library to the C12Cre cells and Dox/Tet and Ad5 stimulation, FACS enrichment was performed (double positive cells—mCherry/GFP) and DNA was isolated for amplification of Cre-flipped DNA. Amplified DNA, plasmid, and viral libraries were barcoded and subjected to deep sequencing (˜100 reads per Kozak variant for plasmid and viral libraries, ˜20 reads per Kozak variant for flipped DNA).

To characterize packaging, the ratio between the number of reads per variant from viral to plasmid libraries was used:

$$Packaging = \frac{\begin{matrix}number of reads Kozak variant viral library / total \\ number of reads from viral library\end{matrix}}{\begin{matrix}number of reads Kozak variant plasmid \\ library / total number of reads from plasmid library\end{matrix}}$$

To characterize infectivity, the ratio between number of reads per variant from flipped DNA to viral library was used:

$$Infectivity = \frac{\begin{matrix}number of reads Kozak variant flipped DNA / total \\ number of reads from flipped DNA\end{matrix}}{\begin{matrix}number of reads Kozak variant viral library / total \\ number of reads from viral library\end{matrix}}$$

Transductions were carried out in duplicates (“Transduction #1 and Transduction #2”) to verify reproducibility of the approach.

Structural-functional analysis of an AAV-Kozak library with a complexity of 65,536 variants for serotype 2 revealed a motif which exhibited an increased packaging rate—NNNN NTGN ATG GC (SEQ ID NO: 48). This can be explained by the out of frame upstream initiation codons NNNN(A/C/G/T)TGN ATG GC (SEQ ID NO: 48), which lead to a reduction of initiation from the downstream in-frame ATG start codon. Remarkably, the upstream initiation does not deplete the abundance of Cap production because upstream initiation codon has an in-frame stop codon after 16 amino acids, which seems to prevent the depletion of ribosomal initiation.

The approach described herein demonstrated the highly-reproducible detection of variants with improved infectivity. 188 and 186 variants were picked from Transduction #1 and Transduction #2, respectively (under the same packaging-infectivity conditions). 177 total variants were represented by both datasets.

Using the packaging-infectivity rates for each Kozak variant, 5 Kozak variants were selected for subsequent characterization, based on the following criteria: production (measured as viral particles per cell; infectivity measured by FACS; the VP1:VP2:VP3 ratio measured on stain free gel). Among the selected Kozak variants, #14 demostrated an identical infectivity rate to AAV2 produced into HEK293 cells.

Analysis of the packaging-infectivity rates for 65,536 variants in the AAV2-Kozak library revealed relationships between Kozak effiency, VP1 content, and capsid assembly rate. Low efficiency Kozak variants were observed to produce more capsid, but with minimal infectivity. High efficiency Kozak variants were observed to produce highly infective capsids, but with barely traceable capsid yield. Kozak variants within the motif NNNN NTGN ATG GC (SEQ ID NO: 48) were observed to generate an abundant amount of capsids per cell, with an acceptable infectivity rate.

Analysis of the Cap sequences of AAV serotypes 1, 3, 6, 7, 8, 9, rh-10 and DJ revealed similarities with AAV2. The wild-type second amino acid was intentionally preserved in the Cap for the AAV2-Kozak library, because this position plays crucial role in the tuning of Kozak sequence efficiency. For example, GCleads to a stronger initiation that narrows fitness space for Kozak selection, but preserves functional activity of Cap, similarities in sequences, and the same second amino acid codon—GC. Taking into account all these factors, the mining of improved Kozak sequences was expanded to AAV serotypes AAV1, AAV3, AAV6, AAV7, AAV8, AAV9, AAVrh10, and AAV-DJ using next generation libraries based on the identified NNNNNTGN ATG GC (SEQ ID NO: 48) motif with a complexity of 4096 variants. The motif which was preselected for the other libraries (SEQ ID NO: 48) could not be used for AAV4 and AAV5 because of the distinctive amino acid in position 2 which affects upstream Kozak sequences. Accordingly, the AAV4 and AAV5 libraries were synthesized with a complexity of 65,536 variants based on the motifs NNNNNNNN ATG AC (SEQ ID NO: 35) and NNNNNNNN ATG TC (SEQ ID NO: 37).

Low complexity libraries were produced for AAV1, AAV3, AAV6, AAV7, AAV8, AAV9, AAVrh10, AAV-DJ, and high complexity libraries were produced for AAV4 and AAV5 using the aforementioned protocol in Sf9 cells. For transduction of C12Cre cells, multiplicities of infection were used that provided 1-5% of GFP positive cells.

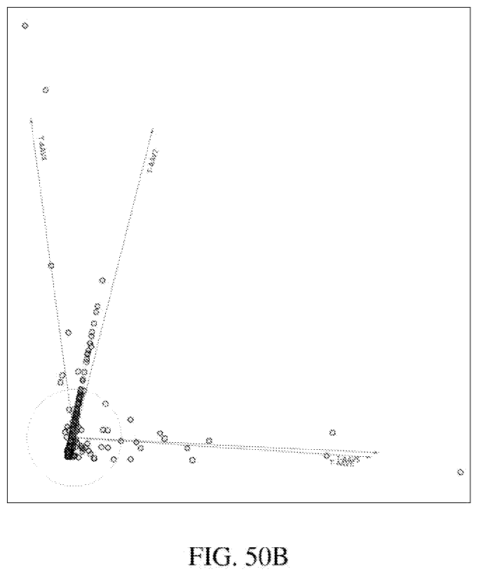

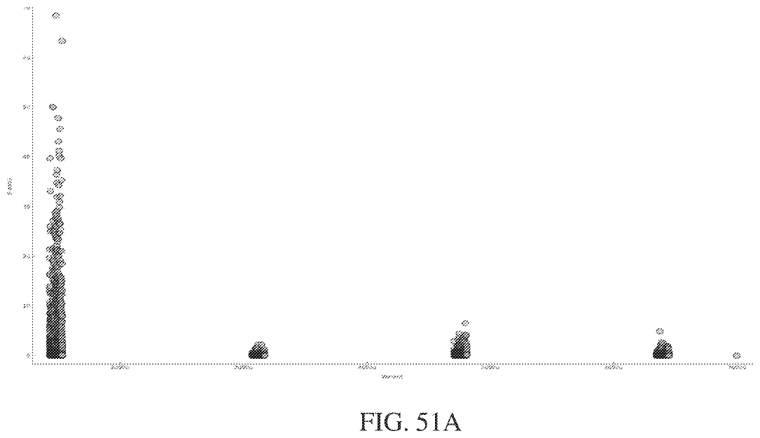

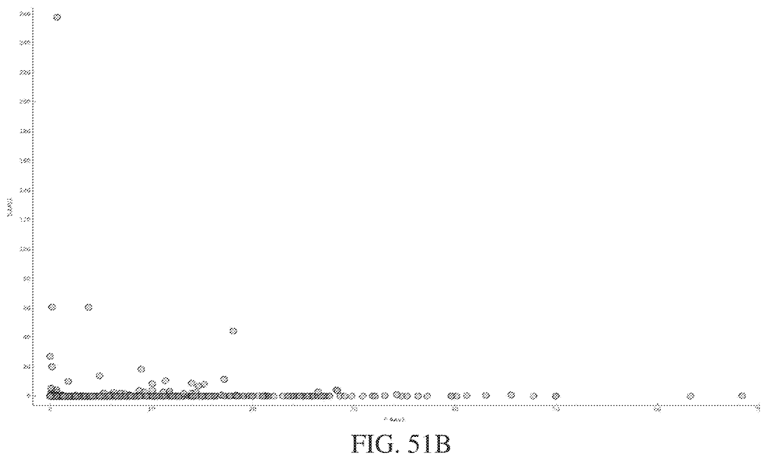

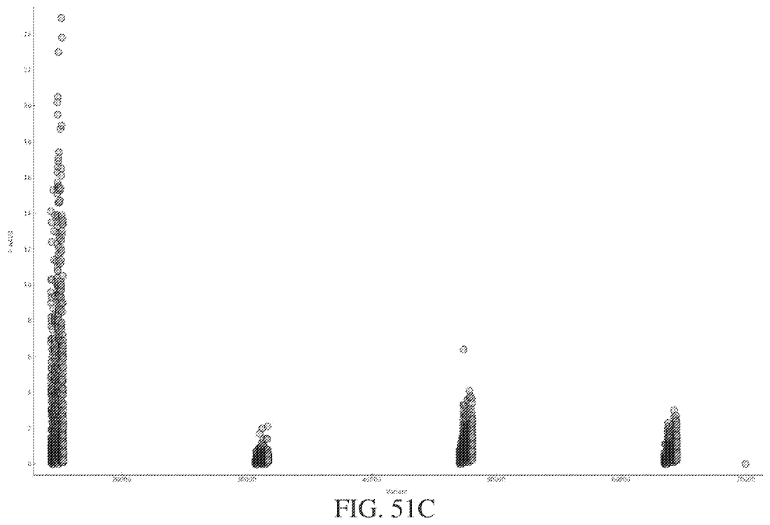

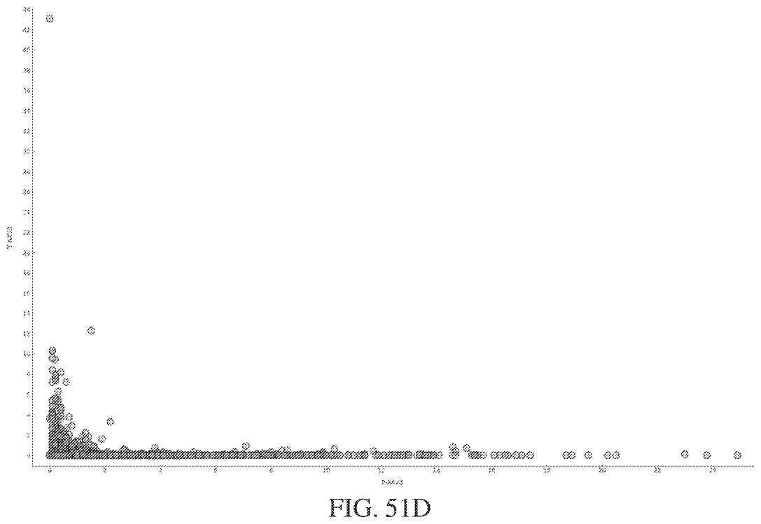

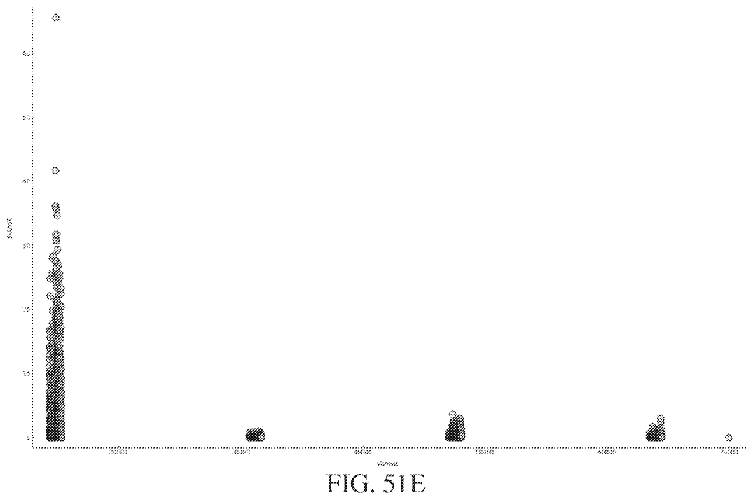

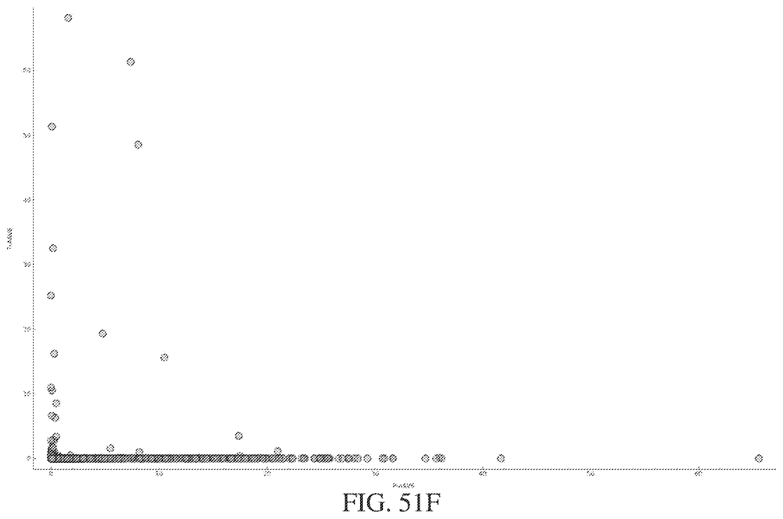

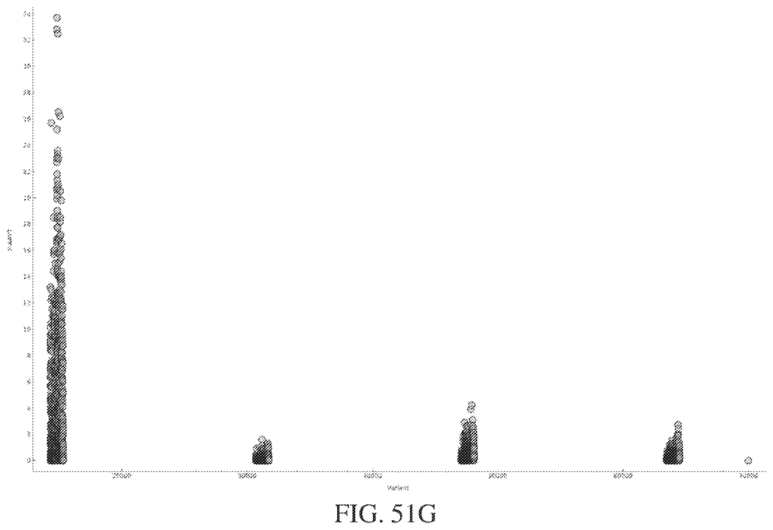

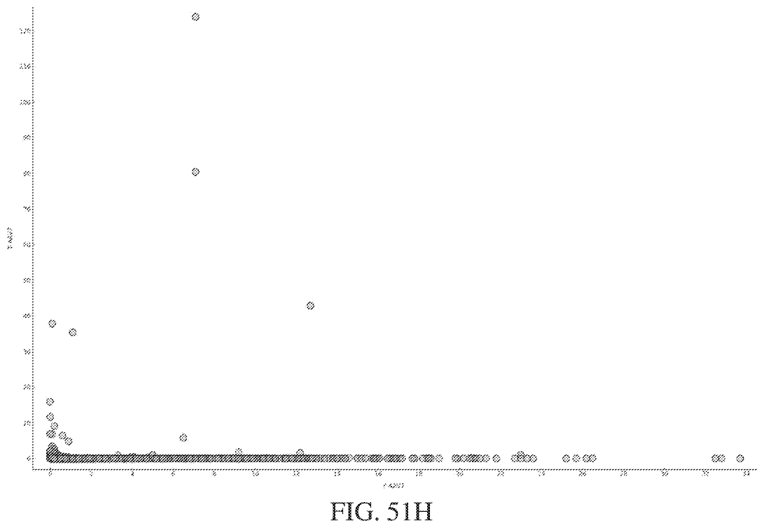

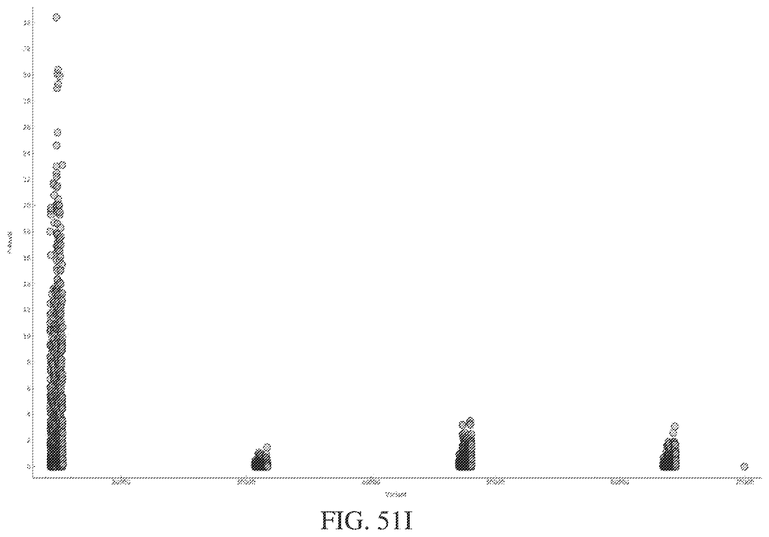

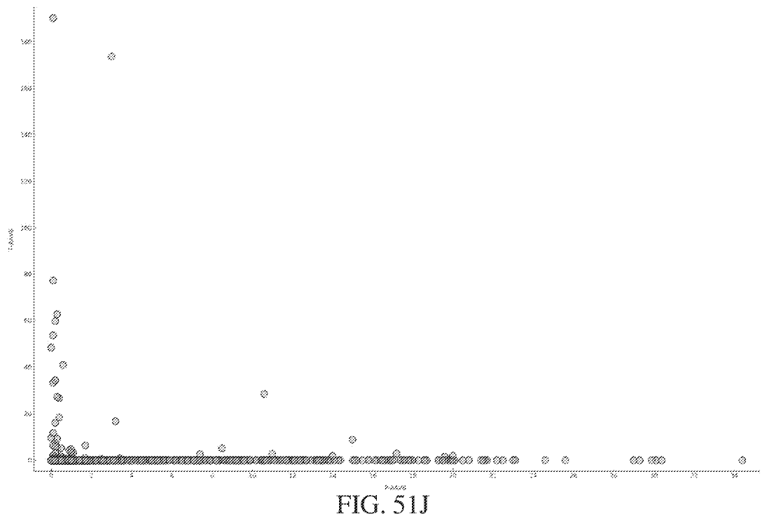

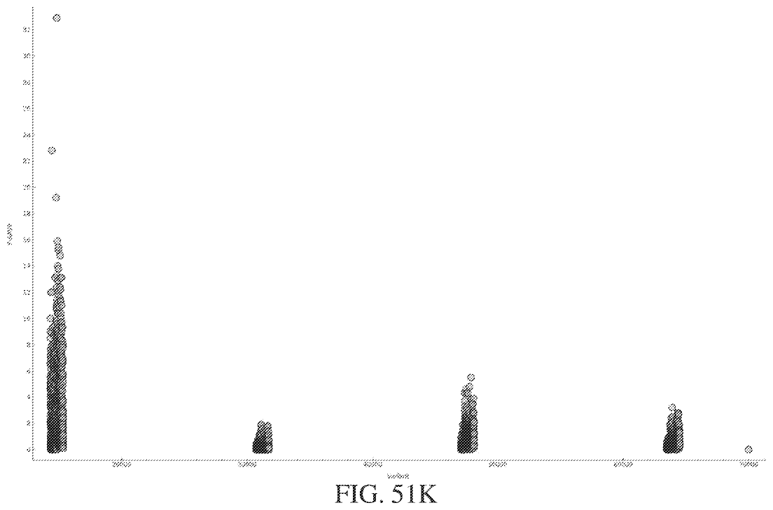

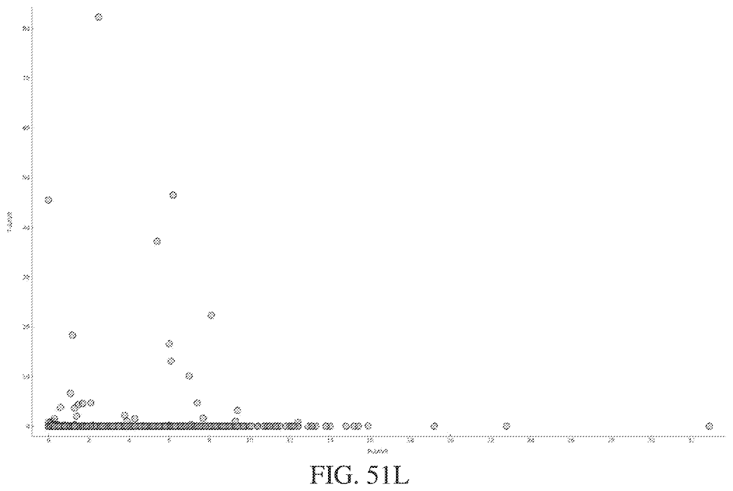

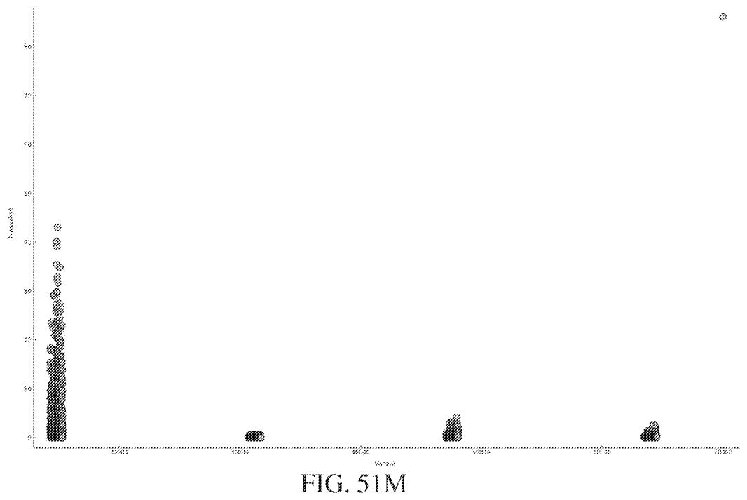

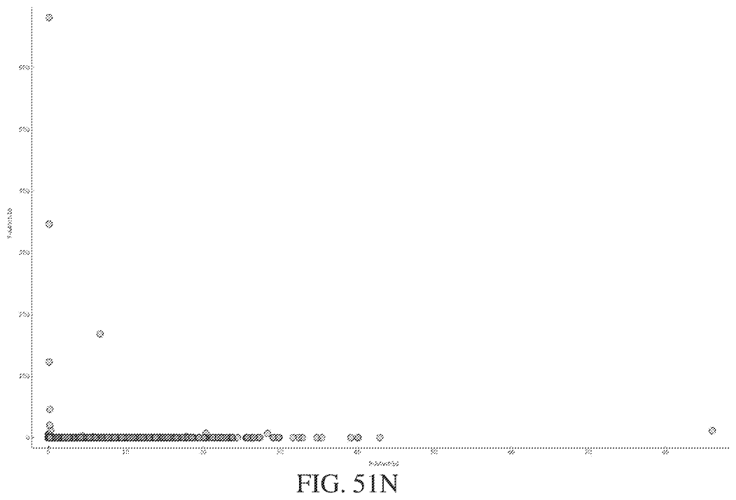

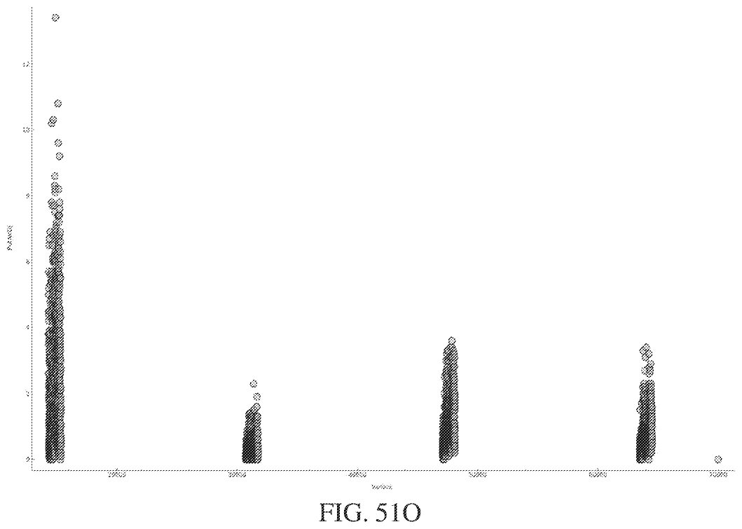

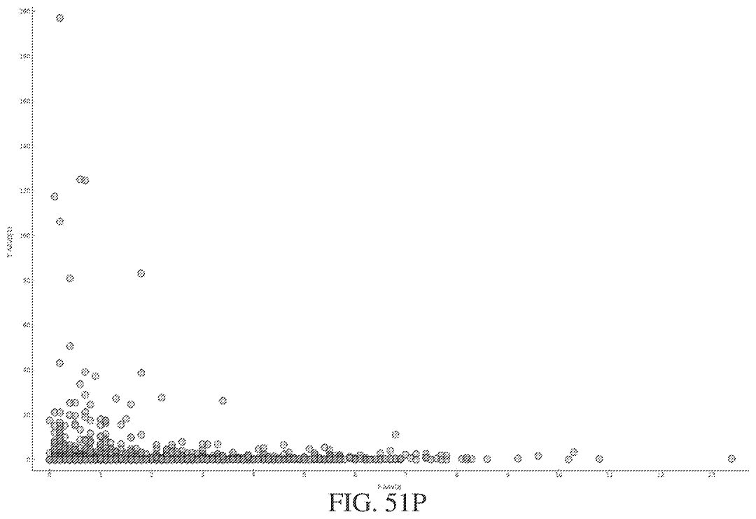

Analysis of packaging-infectivity rates for AAV1, AAV3, AAV4, AAV5, AAV6, AAV7, AAV8, AAV9, AAVrh10 and AAV-DJ revealed significant correlation of packaging rates between and among structurally similar AAV serotypes (R=0.83±0.05): AAV1, AAV3, AAV6, AAV7, AAV8, AAV9, AAVrh10, and AAV-DJ (Table 4), (FIGS. 49A-49B). Also, the AAV4 and AAV5 libraries demonstrated very tight correlation of packaging (R=0.85). These similarities in packaging fitness allowed for the identification of certain variants that share high packaging rate and infectivity among multiple variants: ACCAATGGATGGC (SEQ ID NO: 51), GACCATGCATGGCT (SEQ ID NO: 52), AAAAATGGATGGC (SEQ ID NO: 53), AGTCATGTATGGC (SEQ ID NO: 54), AAACATGGATGGC (SEQ ID NO: 55). Also, a gradual refinement of packaging-infectivity data based on infectivity among several serotypes was completed using Principal Component Analysis (PCA) (FIGS. 50A-50B). Using packaging-infectivity rates that were verified for AAV2 libraries, coupled with PCA analysis, improved Kozak variants were selected for AAV1, AAV3, AAV4, AAV5, AAV6, AAV7, AAV8, AAV9, AAVrh10 and AAV-DJ (see FIGS. 51A-51P).

The library number corresponding to the improved Kozak variants selected for each serotype are shown in Table 4.

TABLE 4
Improved Kozak variants selected for
each serotype, by library number.

| | Library | |
|---|---|---|
| | number of | |
| AAV | identified | |
| Sero- | Kozak | Sequence of identified |
| type | variant | Kozak variant |
| AAV1 | 14594 | AAACATGCATGGC (SEQ ID NO: 56) |
| | 14695 | CGCGATGCATGGC (SEQ ID NO: 57) |
| | 14405 | CACAATGAATGGC (SEQ ID NO: 58) |
| | 14869 | ACCAATGGATGGC (SEQ ID NO: 51) |
| | 14629 | AGCAATGCATGGC (SEQ ID NO: 59) |
| | 14726 | GACCATGCATGGC (SEQ ID NO: 60) |
| | 14889 | AGGAATGGATGGC (SEQ ID NO: 61) |
| | 14863 | AATGATGGATGGC (SEQ ID NO: 62) |
| | 14895 | AGTGATGGATGGC (SEQ ID NO: 63) |
| | 15181 | CATAATGTATGGC (SEQ ID NO: 64) |
| AAV2 | 15005 | GCTAATGGATGGC (SEQ ID NO: 65) |
| | 15298 | TAACATGTATGGC (SEQ ID NO: 66) |
| | 14985 | GAGAATGGATGGC (SEQ ID NO: 67) |
| | 14958 | CGTCATGGATGGC (SEQ ID NO: 68) |
| | 14884 | AGATATGGATGGC (SEQ ID NO: 69) |
| | 14941 | CCTAATGGATGGC (SEQ ID NO: 70) |
| | 29701 | AACACTCAATGGC (SEQ ID NO: 13) |
| | 37235 | CTAGGCACATGGC (SEQ ID NO: 71) |
| AAV3 | 15043 | TAAGATGGATGGC (SEQ ID NO: 72) |
| | 14959 | CGTGATGGATGGC (SEQ ID NO: 73) |
| | 15009 | GGAAATGGATGGC (SEQ ID NO: 74) |
| | 14339 | AAATATGAATGGC (SEQ ID NO: 75) |
| | 14989 | GATAATGGATGGC (SEQ ID NO: 76) |
| | 14849 | AAAAATGGATGGC (SEQ ID NO: 77) |
| AAV4 | 14537 | TAGAATGAATGAC (SEQ ID NO: 78) |
| | 14818 | TGACATGCATGAC (SEQ ID NO: 79) |
| | 14689 | CGAAATGCATGAC (SEQ ID NO: 80) |
| | 14986 | GAGCATGGATGAC (SEQ ID NO: 81) |
| | 15334 | TGCCATGTATGAC (SEQ ID NO: 82) |
| | 14405 | CACAATGAATGAC (SEQ ID NO: 83) |
| AAV5 | 14434 | CGACATGAATGTC (SEQ ID NO: 84) |
| | 14891 | AGGGATGGATGTC (SEQ ID NO: 85) |
| | 14975 | CTTGATGGATGTC (SEQ ID NO: 86) |
| | 15215 | CGTGATGTATGTC (SEQ ID NO: 87) |
| | 15015 | GGCGATGGATGTC (SEQ ID NO: 88) |
| | 14959 | CGTGATGGATGTC (SEQ ID NO: 89) |
| | 14701 | CGTAATGCATGTC (SEQ ID NO: 90) |
| | 14466 | GAACATGAATGTC (SEQ ID NO: 91) |
| | 14754 | GGACATGCATGTC (SEQ ID NO: 92) |
| | 14345 | AAGAATGAATGTC (SEQ ID NO: 93) |
| | 14935 | CCCGATGGATGTC (SEQ ID NO: 94) |
| | 15255 | GCCGATGTATGTC (SEQ ID NO: 95) |
| | 14443 | CGGGATGAATGTC (SEQ ID NO: 96) |
| | 14726 | GACCATGCATGGCT (SEQ ID NO: 97) |
| AAV6 | 14931 | CCAGATGGATGGC (SEQ ID NO: 98) |
| | 15240 | GACTATGTATGGC (SEQ ID NO: 99) |
| | 14890 | AGGCATGGATGGC (SEQ ID NO: 100) |
| | 15215 | CGTGATGTATGGC (SEQ ID NO: 101) |
| | 15053 | TATAATGGATGGC (SEQ ID NO: 102) |
| | 15150 | AGTCATGTATGGC (SEQ ID NO: 54) |
| | 14663 | CACGATGCATGGC (SEQ ID NO: 103) |
| | 14210 | GAACATCTATGGC (SEQ ID NO: 104) |
| | 14726 | GACCATGCATGGC (SEQ ID NO: 60) |
| AAV7 | 47773 | GCTAGTGGATGGC (SEQ ID NO: 105) |
| | 14869 | ACCAATGGATGGC (SEQ ID NO: 51) |
| | 14661 | CACAATGCATGGC (SEQ ID NO: 106) |
| | 14883 | AGAGATGGATGGC (SEQ ID NO: 107) |
| | 15299 | TAAGATGTATGGC (SEQ ID NO: 108) |
| | 15150 | AGTCATGTATGGC (SEQ ID NO: 54) |
| | 14888 | AGCTATGGATGGC (SEQ ID NO: 109) |
| AAV8 | 64471 | TCCGTTGTATGGC (SEQ ID NO: 110) |
| | 47375 | AATGGTGCATGGC (SEQ ID NO: 111) |
| | 14603 | AAGGATGCATGGC (SEQ ID NO: 112) |
| | 14850 | AAACATGGATGGC (SEQ ID NO: 113) |
| | 15142 | AGCCATGTATGGC (SEQ ID NO: 114) |
| AAV9 | 14608 | AATTATGCATGGC (SEQ ID NO: 115) |
| | 15138 | AGACATGTATGGC (SEQ ID NO: 116) |
| | 15113 | AAGAATGTATGGC (SEQ ID NO: 117) |
| | 14726 | GACCATGCATGGCT (SEQ ID NO: 52) |
| | 14951 | CGCGATGGATGGC (SEQ ID NO: 118) |
| | 14887 | AGCGATGGATGGC (SEQ ID NO: 119) |
| | 14734 | GATCATGCATGGC (SEQ ID NO: 120) |
| | 14957 | CGTAATGGATGGC (SEQ ID NO: 121) |
| | 15111 | AACGATGTATGGC (SEQ ID NO: 122) |
| AAV- | 14851 | AAAGATGGATGGC (SEQ ID NO: 123) |
| rh10 | | |
| | 14915 | CAAGATGGATGGC (SEQ ID NO: 124) |
| | 14849 | AAAAATGGATGGC (SEQ ID NO: 77) |
| AAV- | 14855 | AACGATGGATGGC (SEQ ID NO: 125) |
| DJ | | |
| | 15080 | TGCTATGGATGGC (SEQ ID NO: 126) |
| | 15238 | GACCATGTATGGC (SEQ ID NO: 127) |
| | 15245 | GATAATGTATGGC (SEQ ID NO: 128) |

Additionally, variant 31400 (GGCTCTGGATGGC (SEQ ID NO: 129)) was selected as a multi-AAV efficient Kozak variant (e.g., a variant which was efficient among several AAV serotypes).

FIGS. 49A-51P illustrate the illustrate the development and empirical testing of the combinatorial plasmid libraries described in Example 2, from which certain modified Kozak sequences (see Table 4) were identified according to the methods described herein.

The correlation of the package rate between various AAV serotypes for low complexity libraries is shown in Table 5.

TABLE 5
Correlation of packaging rate (Kozak variant, by AAV variant) for low
complexity libraries: AAV1, AAV3, AAV4, AAV5, AAV6, AAV7,
AAV8, AAV9, AAVrh10, and AAV-DJ.

| Correlation | Packaging 1 | Packaging 2 |
|---|---|---|
| 0.92 | P-AAV6 | P-AAV7 |
| 0.91 | P-AAV7 | P-AAV8 |
| 0.91 | P-AAV6 | P-AAV8 |
| 0.90 | P-AAV3 | P-AAV7 |
| 0.89 | P-AAV3 | P-AAV8 |
| 0.88 | P-AAV3 | P-AAV6 |
| 0.87 | P-AAV7 | P-AAVrh10 |
| 0.86 | P-AAV6 | P-AAVrh10 |
| 0.86 | P-AAV9 | P-AAVDJ |
| 0.86 | P-AAV8 | P-AAVrh10 |
| 0.84 | P-AAV7 | P-AAV9 |
| 0.84 | P-AAV8 | P-AAV9 |
| 0.83 | P-AAV7 | P-AAVDJ |
| 0.83 | P-AAV8 | P-AAVDJ |
| 0.82 | P-AAV3 | P-AAVDJ |
| 0.82 | P-AAV3 | P-AAV9 |
| 0.82 | P-AAV3 | P-AAVrh10 |
| 0.81 | P-AAV1 | P-AAV7 |
| 0.81 | P-AAV1 | P-AAV8 |
| 0.80 | P-AAV6 | P-AAV9 |
| 0.79 | P-AAV1 | P-AAV6 |
| 0.78 | P-AAV6 | P-AAVDJ |
| 0.77 | P-AAV9 | P-AAVrh10 |
| 0.77 | P-AAV1 | P-AAV3 |
| 0.76 | P-AAV1 | P-AAVrh10 |
| 0.75 | P-AAVDJ | P-AAVrh10 |
| 0.73 | P-AAV1 | P-AAV9 |
| 0.73 | P-AAV1 | P-AAVDJ |
| Average | StDev | |
| 0.83 | 0.05 | |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be non-limiting and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A nucleic acid comprising a nucleotide sequence encoding a modified Kozak sequence and adeno-associated virus (AAV) VP1, VP2, and VP3 capsid proteins, wherein the modified Kozak sequence is selected from AACGATGCATGGC (SEQ ID NO: 14), CAACATGAATGGC (SEQ ID NO: 16), GATCATGGATGGC (SEQ ID NO: 17), or CGCGATGGATGGC (SEQ ID NO: 22).

2. The nucleic acid of claim 1, wherein the modified Kozak sequence is AACGATGCATGGC (SEQ ID NO: 14).

3. The nucleic acid of claim 1, wherein the modified Kozak sequence is CAACATGAATGGC (SEQ ID NO: 16).

4. The nucleic acid of claim 1, wherein the modified Kozak sequence is GATCATGGATGGC (SEQ ID NO: 17).

5. The nucleic acid of claim 1, wherein the modified Kozak sequence is CGCGATGGATGGC (SEQ ID NO: 22).

6. The nucleic acid of any one of claims 1-5, wherein the VP1, VP2, and VP3 capsid proteins are from an AAV2 serotype.

7. The nucleic acid of any one of claims 1-5, wherein the VP1, VP2, and VP3 capsid proteins are from an AAV1, AAV2, AAV3, AAV4, AAV5, AAV6, AAV7, AAV8, AAV9, AAV-rh10, or AAV-DJ serotype.

8. The nucleic acid of any one of claims 1-7, further comprising a promoter sequence.

9. The nucleic acid of claim 8, wherein the promoter sequence is a polyhedrin (polh) promoter sequence.

10. The nucleic acid of any one of claims 1-9, wherein the modified Kozak sequence contains an initiation codon for translation of the VP1 capsid protein.

11. The nucleic acid of claim 10, wherein the initiation codon for translation of the VP1 capsid protein is ATG.

12. The nucleic acid of any one of claims 1-9, wherein the modified Kozak sequence contains an initiation codon for translation of the VP2 capsid protein.

13. The nucleic acid of claim 12, wherein the initiation codon for translation of the VP2 capsid protein is ATG.

14. The nucleic acid of any one of claims 1-9, wherein the modified Kozak sequence contains an initiation codon for translation of the VP3 capsid protein.

15. The nucleic acid of claim 14, wherein the initiation codon for translation of the VP3 capsid protein is ATG.

16. A baculovirus particle comprising the nucleic acid of any one of claims 1-15.

17. An insect cell comprising the nucleic acid of any one of claims 1-15.

18. The insect cell of claim 17, wherein the nucleic acid is integrated into the genome of the insect cell.

19. A method of producing and screening recombinant AAV (rAAV) comprising:
a) transfecting a first insect cell with:
i) a baculovirus comprising the nucleic acid of any one of claims 1-15;
ii) a baculovirus comprising a nucleic acid encoding an AAV Rep protein;
iii) a baculovirus comprising a nucleic acid comprising two AAV inverted terminal repeat (ITR) nucleotide sequences flanking a nucleic acid encoding an AAV Cap protein operably linked to a promoter sequence;
b) culturing the first insect cell under conditions suitable to produce the rAAV;
c) recovering the rAAV from the insect cell;
d) infecting a mammalian cell with the recovered rAAV; and
e) extracting DNA from the mammalian cell.

20. The method of claim 19 further comprising:
f) amplifying the nucleotide sequence encoding a modified Kozak sequence from the extracted DNA; and
g) subcloning the amplified modified Kozak sequences into an AAV plasmid.

21. The method of claim 20 further comprising:
h) transfecting a second insect cell with a baculovirus comprising the AAV plasmid of step (g).

22. The method of any one of claims 19-21, wherein the multiplicity of infection (MOI) of step (a) is about 2, about 3, about 4, about 5, about 10, or about 20.

23. The method of claim 22, wherein the MOI of step a) is about 3.

24. The method of any one of claims 19-23, wherein the multiplicity of infection (MOI) of step d) is about 1000.

25. The method of any one of claims 19-24, wherein the first and/or second insect cell is an Sf9 cell.

26. The method of any one of claims 19-25, wherein the mammalian cell is an HEK293T cell.

27. The method of any one of claims 19-26, wherein the rAAV is derived from an AAV2 serotype.

28. The method of any one of claims 19-26, wherein the rAAV is derived from an AAV1, AAV2, AAV3, AAV4, AAV5, AAV6, AAV7, AAV8, AAV9, AAV-rh10, or AAV-DJ serotype.

29. The nucleic acid of any one of claims 1-15, wherein the modified Kozak sequence is AACGATGCATGGC (SEQ ID NO: 14), and wherein the ratio of VP1:VP2:VP3 is not 1:1:1.

30. The nucleic acid of any one of claims 1-15, wherein the modified Kozak sequence is AACGATGCATGGC (SEQ ID NO: 14), and wherein the ratio of VP1:VP2:VP3 is 1:0.3:9.

31. The nucleic acid of any one of claims 1-15, wherein the modified Kozak sequence is CAACATGAATGGC (SEQ ID NO: 16), and wherein the ratio of VP1:VP2:VP3 is not 1:1:1.

32. The nucleic acid of any one of claims 1-15, wherein the modified Kozak sequence is CAACATGAATGGC (SEQ ID NO: 16), and wherein the ratio of VP1:VP2:VP3 is 1:0.3:8.

33. The nucleic acid of any one of claims 1-15, wherein the modified Kozak sequence is GATCATGGATGGC (SEQ ID NO: 17), and wherein the ratio of VP1:VP2:VP3 is not 1:1:1.

34. The nucleic acid of any one of claims 1-15, wherein the modified Kozak sequence is GATCATGGATGGC (SEQ ID NO: 17), and wherein the ratio of VP1:VP2:VP3 is 1:0.6:9.

35. The nucleic acid of any one of claims 1-15, wherein the modified Kozak sequence is CGCGATGGATGGC (SEQ ID NO: 22), and wherein the ratio of VP1:VP2:VP3 is not 1:1:1.

36. The nucleic acid of any one of claims 1-15, wherein the modified Kozak sequence is CGCGATGGATGGC (SEQ ID NO: 22), and wherein the ratio of VP1:VP2:VP3 is 1:0.4:14.

37. A method of producing an rAAV particle by contacting an insect cell with a baculovirus comprising a capsid gene containing the nucleic acid of any one of claims 1-15 and 29-36.

38. The method of claim 37, wherein the insect cell is an Sf9 cell.

39. A method of producing an rAAV particle by contacting a mammalian cell with a nucleic acid comprising a capsid gene containing the nucleic acid of any one of claims 1-15 and 29-36.

40. The method of claim 39, wherein the mammalian cell is an HEK293T cell.

41. The method of any one of claims 37-40, wherein the rAAV particle is derived from an AAV1, AAV2, AAV3, AAV4, AAV5, AAV6, AAV7, AAV8, AAV9, AAV-rh10, or AAV-DJ serotype.

42. The method of any one of claims 37-41, wherein the rAAV particle displays characteristics of potency that are equivalent to an rAAV produced in mammalian cells using the same AAV serotype and the wild-type Kozak sequence.

43. The method of claim 48, wherein the characteristics of potency are multiplicity of infection, transduction efficiency, and/or production yield.

44. An rAAV particle produced according to any of the methods of claims 37-43, wherein the rAAV particle comprises a nucleic acid encoding a gene of interest.

45. An rAAV particle comprising a protein encoded by the nucleic acid of any one of claims 1-15 and 29-36, and a nucleic acid encoding a gene of interest.

46. The rAAV particle of claim 44 or 45, wherein the rAAV is derived from an AAV2 serotype.

47. The rAAV particle of claim 44 or 45, wherein the rAAV is derived from an AAV1, AAV2, AAV3, AAV4, AAV5, AAV6, AAV7, AAV8, AAV9, AAV-rh10, or AAV-DJ serotype.

48. A mammalian cell comprising the nucleic acid of any one of claims 1-15 and 29-36, or the rAAV particle of any one of claims 44-47.

49. A method of treatment of a subject suffering from a disease, disorder or condition comprising administering to the subject the rAAV particle of any one of claims 44-47 or the mammalian cell of claim 48.

50. The method of claim 49, wherein the subject is a human subject.

51. The rAAV particle of any one of claims 44-47, wherein the gene of interest encodes a protein or polypeptide of interest, and wherein the protein or polypeptide of interest is recited in Table 1.

52. The rAAV particle of any one of claims 44-47, wherein the gene of interest encodes a protein or polypeptide of interest, wherein the protein or polypeptide of interest is a clotting factor, globin, adrenergic agonist, anti-apoptosis factor, apoptosis inhibitor, cytokine receptor, cytokine, cytotoxin, erythropoietic agent, glutamic acid decarboxylase, glycoprotein, growth factor, growth factor receptor, hormone, hormone receptor, interferon, interleukin, interleukin receptor, kinase, kinase inhibitor, nerve growth factor, netrin, neuroactive peptide, neuroactive peptide receptor, neurogenic factor, neurogenic factor receptor, neuropilin, neurotrophic factor, neurotrophin, neurotrophin receptor, N-methyl-D-aspartate antagonist, plexin, protease, protease inhibitor, protein decarboxylase, protein kinase, protein kinsase inhibitor, proteolytic protein, proteolytic protein inhibitor, semaphoring, semaphorin receptor, serotonin transport protein, serotonin uptake inhibitor, serotonin receptor, serpin, serpin receptor, or tumor suppressor.

53. A nucleic acid comprising a nucleotide sequence encoding a modified Kozak sequence and adeno-associated virus (AAV) VP1, VP2, and VP3 capsid proteins, wherein the modified Kozak sequence is a nucleotide sequence comprising no more than 1, 2, or 3 nucleic acid variations relative to a nucleotide sequence selected from any one of SEQ ID NOs: 13-14, 16-17, 22, 51-52, 54, and 56-129.

54. The nucleic acid of claim 53, wherein the modified Kozak sequence is a nucleotide sequence selected from any one of SEQ ID NOs: 13-14, 16-17, 22, 51-52, 54, and 56-129.

55. The nucleic acid of claim 53, wherein the modified Kozak sequence is a nucleotide sequence selected from any one of SEQ ID NOs: 65-71.

56. The nucleic acid of any one of claims 53-55, wherein the VP1, VP2, and VP3 capsid proteins are from an AAV2 serotype.

57. The nucleic acid of claim 54, wherein the modified Kozak sequence is a nucleotide sequence selected from any one of SEQ ID NOs: 52, 72-77, 78-83, 84-97, 115-117, and 119-122.

58. The nucleic acid of claim 57, the VP1, VP2, and VP3 capsid proteins are from an AAV3, AAV4, AAV5, or AAV9 serotype.

59. The nucleic acid of any one of claims 53-58, wherein the VP1, VP2, and VP3 capsid proteins are from an AAV1, AAV2, AAV3, AAV4, AAV5, AAV6, AAV7, AAV8, AAV9, AAV-rh10, and AAV-DJ serotype.


SEQUENCE LISTING (129 sequences)

SEQ ID NO: 1 - DNA, 13 bp, Artificial Sequence
  Synthetic
ccgccctcatggc

SEQ ID NO: 2 - DNA, 13 bp, Artificial Sequence
  Synthetic
gggccgcaatggc

SEQ ID NO: 3 - DNA, 13 bp, Artificial Sequence
  Synthetic
cagtggctatggc

SEQ ID NO: 4 - DNA, 13 bp, Artificial Sequence
  Synthetic
agttactaatggc

SEQ ID NO: 5 - DNA, 13 bp, Artificial Sequence
  Synthetic
gaagagcgatggc

SEQ ID NO: 6 - DNA, 13 bp, Artificial Sequence
  Synthetic
acgccaatatggc

SEQ ID NO: 7 - DNA, 13 bp, Artificial Sequence
  Synthetic
ctttggacatggc

SEQ ID NO: 8 - DNA, 13 bp, Artificial Sequence
  Synthetic
aggcaatcatggc

SEQ ID NO: 9 - DNA, 13 bp, Artificial Sequence
  Synthetic
cgaccgccatggc

SEQ ID NO: 10 - DNA, 13 bp, Artificial Sequence
  Synthetic
acgcctgtatggc

SEQ ID NO: 11 - DNA, 13 bp, Artificial Sequence
  Synthetic
acaaattcatggc

SEQ ID NO: 12 - DNA, 13 bp, Artificial Sequence
  Synthetic
ttcctctaatggc

SEQ ID NO: 13 - DNA, 13 bp, Artificial Sequence
  Synthetic
aacactcaatggc

SEQ ID NO: 14 - DNA, 13 bp, Artificial Sequence
  Synthetic
aacgatgcatggc

SEQ ID NO: 15 - DNA, 13 bp, Artificial Sequence
  Synthetic
cagaatggatggc

SEQ ID NO: 16 - DNA, 13 bp, Artificial Sequence
  Synthetic
caacatgaatggc

SEQ ID NO: 17 - DNA, 13 bp, Artificial Sequence
  Synthetic
gatcatggatggc

SEQ ID NO: 18 - DNA, 13 bp, Artificial Sequence
  Synthetic
gcttggagatggc

SEQ ID NO: 19 - DNA, 13 bp, Artificial Sequence
  Synthetic
gaacatgtatggc

SEQ ID NO: 20 - DNA, 13 bp, Artificial Sequence
  Synthetic
gaatatggatggc

SEQ ID NO: 21 - DNA, 13 bp, Artificial Sequence
  Synthetic
tagattgaatggc

SEQ ID NO: 22 - DNA, 13 bp, Artificial Sequence
  Synthetic
cgcgatggatggc

SEQ ID NO: 23 - DNA, 13 bp, Artificial Sequence
  Synthetic
cgcaatggatggc

SEQ ID NO: 24 - DNA, 13 bp, Artificial Sequence
  Synthetic
ggcaatggatggc

SEQ ID NO: 25 - DNA, 13 bp, Artificial Sequence
  Synthetic
ttgatccaatggc

SEQ ID NO: 26 - DNA, 13 bp, Artificial Sequence
  Synthetic
caaaatggatggc

SEQ ID NO: 27 - DNA, 13 bp, Artificial Sequence
  Synthetic
cccggtgtatggc

SEQ ID NO: 28 - DNA, 13 bp, Artificial Sequence
  Synthetic
ataccgacagggc

SEQ ID NO: 29 - DNA, 13 bp, Artificial Sequence
  Synthetic
ttcatgacatagc

SEQ ID NO: 30 - DNA, 13 bp, Artificial Sequence
  Synthetic
gttttcttagggc

SEQ ID NO: 31 - DNA, 13 bp, Artificial Sequence
  Synthetic
gtaacgttatagc

SEQ ID NO: 32 - DNA, 13 bp, Artificial Sequence
  Synthetic
actttaaaacggc

SEQ ID NO: 33 - DNA, 13 bp, Artificial Sequence
  Synthetic
  misc_feature (1)..(4): n is a, c, g, or t
  misc_feature (8)..(8): n is a, c, g, or t
nnnnatgnatggc

SEQ ID NO: 34 - DNA, 13 bp, Artificial Sequence
  Synthetic
  misc_feature (1)..(4): n is a, c, g, or t
  misc_feature (8)..(8): n is a, c, g, or t
nnnnatgnatggc

SEQ ID NO: 35 - DNA, 13 bp, Artificial Sequence
  Synthetic
  misc_feature (1)..(8): n is a, c, g, or t
nnnnnnnnatgac

SEQ ID NO: 36 - DNA, 13 bp, Artificial Sequence
  Synthetic
  misc_feature (1)..(8): n is a, c, g, or t
nnnnnnnnatgtc

SEQ ID NO: 37 - DNA, 13 bp, Artificial Sequence
  Synthetic
  misc_feature (1)..(4): n is a, c, g, or t
  misc_feature (8)..(8): n is a, c, g, or t
nnnnatgnatggc

SEQ ID NO: 38 - DNA, 13 bp, Artificial Sequence
  Synthetic
  misc_feature (1)..(4): n is a, c, g, or t
  misc_feature (8)..(8): n is a, c, g, or t
nnnnatgnatggc

SEQ ID NO: 39 - DNA, 13 bp, Artificial Sequence
  Synthetic
  misc_feature (1)..(4): n is a, c, g, or t
  misc_feature (8)..(8): n is a, c, g, or t
nnnnatgnatggc

SEQ ID NO: 40 - DNA, 13 bp, Artificial Sequence
  Synthetic
  misc_feature (1)..(4): n is a, c, g, or t
  misc_feature (8)..(8): n is a, c, g, or t
nnnnatgnatggc

SEQ ID NO: 41 - DNA, 13 bp, Artificial Sequence
  Synthetic
  misc_feature (1)..(4): n is a, c, g, or t
  misc_feature (8)..(8): n is a, c, g, or t
nnnnatgnatggc

SEQ ID NO: 42 - DNA, 13 bp, Artificial Sequence
  Synthetic
  misc_feature (1)..(4): n is a, c, g, or t
  misc_feature (8)..(8): n is a, c, g, or t
nnnnatgnatggc

SEQ ID NO: 43 - protein, 672 aa, Artificial Sequence
  Synthetic
YLGPGNGLGRGEPVNRADEVAREHDISYNEQLEAGDNPYLKYNHADEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTITIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFMSQPANPGTTATYLEGNMLITSESETQPVNRVAYNVGGQMATNNQSSTTAPATGTYNLQEIVPGSVMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 44 - protein, 12 aa, Artificial Sequence
  Synthetic
LANPLVDQYLYR

SEQ ID NO: 45 - protein, 13 aa, Artificial Sequence
  Synthetic
TWVLPSYNNHQYR

SEQ ID NO: 46 - protein, 20 aa, Artificial Sequence
  Synthetic
IPETGAHFHPSPAMGGFGLK

SEQ ID NO: 47 - DNA, 10 bp, Artificial Sequence
  Synthetic
gccgccctgg

SEQ ID NO: 48 - DNA, 13 bp, Artificial Sequence
  Synthetic
  misc_feature (1)..(8): n is a, c, g, or t
nnnnnnnnatggc

SEQ ID NO: 49 - DNA, 11 bp, Artificial Sequence
  Synthetic
  misc_feature (1)..(6): n is a, c, g, or t
  misc_feature (10)..(11): n is a, c, g, or t
nnnnnnatgnn

SEQ ID NO: 50 - protein, 13 aa, Artificial Sequence
  Synthetic
TWVLPSYNNHQYR

SEQ ID NO: 51 - DNA, 13 bp, Artificial Sequence
  Synthetic
accaatggatggc

SEQ ID NO: 52 - DNA, 14 bp, Artificial Sequence
  Synthetic
gaccatgcatggct

SEQ ID NO: 53 - DNA, 13 bp, Artificial Sequence
  Synthetic
aaaaatggatggc

SEQ ID NO: 54 - DNA, 13 bp, Artificial Sequence
  Synthetic
agtcatgtatggc

SEQ ID NO: 55 - DNA, 13 bp, Artificial Sequence
  Synthetic
aaacatggatggc

SEQ ID NO: 56 - DNA, 13 bp, Artificial Sequence
  Synthetic
aaacatgcatggc

SEQ ID NO: 57 - DNA, 13 bp, Artificial Sequence
  Synthetic
cgcgatgcatggc

SEQ ID NO: 58 - DNA, 13 bp, Artificial Sequence
  Synthetic
cacaatgaatggc

SEQ ID NO: 59 - DNA, 13 bp, Artificial Sequence
  Synthetic
agcaatgcatggc

SEQ ID NO: 60 - DNA, 13 bp, Artificial Sequence
  Synthetic
gaccatgcatggc

SEQ ID NO: 61 - DNA, 13 bp, Artificial Sequence
  Synthetic
aggaatggatggc

SEQ ID NO: 62 - DNA, 13 bp, Artificial Sequence
  Synthetic
aatgatggatggc

SEQ ID NO: 63 - DNA, 13 bp, Artificial Sequence
  Synthetic
agtgatggatggc

SEQ ID NO: 64 - DNA, 13 bp, Artificial Sequence
  Synthetic
cataatgtatggc

SEQ ID NO: 65 - DNA, 13 bp, Artificial Sequence
  Synthetic
gctaatggatggc

SEQ ID NO: 66 - DNA, 13 bp, Artificial Sequence
  Synthetic
taacatgtatggc

SEQ ID NO: 67 - DNA, 13 bp, Artificial Sequence
  Synthetic
gagaatggatggc

SEQ ID NO: 68 - DNA, 13 bp, Artificial Sequence
  Synthetic
cgtcatggatggc

SEQ ID NO: 69 - DNA, 13 bp, Artificial Sequence
  Synthetic
agatatggatggc

SEQ ID NO: 70 - DNA, 13 bp, Artificial Sequence
  Synthetic
cctaatggatggc

SEQ ID NO: 71 - DNA, 13 bp, Artificial Sequence
  Synthetic
ctaggcacatggc

SEQ ID NO: 72 - DNA, 13 bp, Artificial Sequence
  Synthetic
taagatggatggc

SEQ ID NO: 73 - DNA, 13 bp, Artificial Sequence
  Synthetic
cgtgatggatggc

SEQ ID NO: 74 - DNA, 13 bp, Artificial Sequence
  Synthetic
ggaaatggatggc

SEQ ID NO: 75 - DNA, 13 bp, Artificial Sequence
  Synthetic
aaatatgaatggc

SEQ ID NO: 76 - DNA, 13 bp, Artificial Sequence
  Synthetic
gataatggatggc

SEQ ID NO: 77 - DNA, 13 bp, Artificial Sequence
  Synthetic
aaaaatggatggc

SEQ ID NO: 78 - DNA, 13 bp, Artificial Sequence
  Synthetic
tagaatgaatgac

SEQ ID NO: 79 - DNA, 13 bp, Artificial Sequence
  Synthetic
tgacatgcatgac

SEQ ID NO: 80 - DNA, 13 bp, Artificial Sequence
  Synthetic
cgaaatgcatgac

SEQ ID NO: 81 - DNA, 13 bp, Artificial Sequence
  Synthetic
gagcatggatgac

SEQ ID NO: 82 - DNA, 13 bp, Artificial Sequence
  Synthetic
tgccatgtatgac

SEQ ID NO: 83 - DNA, 13 bp, Artificial Sequence
  Synthetic
cacaatgaatgac

SEQ ID NO: 84 - DNA, 13 bp, Artificial Sequence
  Synthetic
cgacatgaatgtc

SEQ ID NO: 85 - DNA, 13 bp, Artificial Sequence
  Synthetic
agggatggatgtc

SEQ ID NO: 86 - DNA, 13 bp, Artificial Sequence
  Synthetic
cttgatggatgtc

SEQ ID NO: 87 - DNA, 13 bp, Artificial Sequence
  Synthetic
cgtgatgtatgtc

SEQ ID NO: 88 - DNA, 13 bp, Artificial Sequence
  Synthetic
ggcgatggatgtc

SEQ ID NO: 89 - DNA, 13 bp, Artificial Sequence
  Synthetic
cgtgatggatgtc

SEQ ID NO: 90 - DNA, 13 bp, Artificial Sequence
  Synthetic
cgtaatgcatgtc

SEQ ID NO: 91 - DNA, 13 bp, Artificial Sequence
  Synthetic
gaacatgaatgtc

SEQ ID NO: 92 - DNA, 13 bp, Artificial Sequence
  Synthetic
ggacatgcatgtc

SEQ ID NO: 93 - DNA, 13 bp, Artificial Sequence
  Synthetic
aagaatgaatgtc

SEQ ID NO: 94 - DNA, 13 bp, Artificial Sequence
  Synthetic
cccgatggatgtc

SEQ ID NO: 95 - DNA, 13 bp, Artificial Sequence
  Synthetic
gccgatgtatgtc

SEQ ID NO: 96 - DNA, 13 bp, Artificial Sequence
  Synthetic
cgggatgaatgtc

SEQ ID NO: 97 - DNA, 14 bp, Artificial Sequence
  Synthetic
gaccatgcatggct

SEQ ID NO: 98 - DNA, 13 bp, Artificial Sequence
  Synthetic
ccagatggatggc

SEQ ID NO: 99 - DNA, 13 bp, Artificial Sequence
  Synthetic
gactatgtatggc

SEQ ID NO: 100 - DNA, 13 bp, Artificial Sequence
  Synthetic
aggcatggatggc

SEQ ID NO: 101 - DNA, 13 bp, Artificial Sequence
  Synthetic
cgtgatgtatggc

SEQ ID NO: 102 - DNA, 13 bp, Artificial Sequence
  Synthetic
tataatggatggc

SEQ ID NO: 103 - DNA, 13 bp, Artificial Sequence
  Synthetic
cacgatgcatggc

SEQ ID NO: 104 - DNA, 13 bp, Artificial Sequence
  Synthetic
gaacatctatggc

SEQ ID NO: 105 - DNA, 13 bp, Artificial Sequence
  Synthetic
gctagtggatggc

SEQ ID NO: 106 - DNA, 13 bp, Artificial Sequence
  Synthetic
cacaatgcatggc

SEQ ID NO: 107 - DNA, 13 bp, Artificial Sequence
  Synthetic
agagatggatggc

SEQ ID NO: 108 - DNA, 13 bp, Artificial Sequence
  Synthetic
taagatgtatggc

SEQ ID NO: 109 - DNA, 13 bp, Artificial Sequence
  Synthetic
agctatggatggc

SEQ ID NO: 110 - DNA, 13 bp, Artificial Sequence
  Synthetic
tccgttgtatggc

SEQ ID NO: 111 - DNA, 13 bp, Artificial Sequence
  Synthetic
aatggtgcatggc

SEQ ID NO: 112 - DNA, 13 bp, Artificial Sequence
  Synthetic
aaggatgcatggc

SEQ ID NO: 113 - DNA, 13 bp, Artificial Sequence
  Synthetic
aaacatggatggc

SEQ ID NO: 114 - DNA, 13 bp, Artificial Sequence
  Synthetic
agccatgtatggc

SEQ ID NO: 115 - DNA, 13 bp, Artificial Sequence
  Synthetic
aattatgcatggc

SEQ ID NO: 116 - DNA, 13 bp, Artificial Sequence
  Synthetic
agacatgtatggc

SEQ ID NO: 117 - DNA, 13 bp, Artificial Sequence
  Synthetic
aagaatgtatggc

SEQ ID NO: 118 - DNA, 13 bp, Artificial Sequence
  Synthetic
cgcgatggatggc

SEQ ID NO: 119 - DNA, 13 bp, Artificial Sequence
  Synthetic
agcgatggatggc

SEQ ID NO: 120 - DNA, 13 bp, Artificial Sequence
  Synthetic
gatcatgcatggc

SEQ ID NO: 121 - DNA, 13 bp, Artificial Sequence
  Synthetic
cgtaatggatggc

SEQ ID NO: 122 - DNA, 13 bp, Artificial Sequence
  Synthetic
aacgatgtatggc

SEQ ID NO: 123 - DNA, 13 bp, Artificial Sequence
  Synthetic
aaagatggatggc

SEQ ID NO: 124 - DNA, 13 bp, Artificial Sequence
  Synthetic
caagatggatggc

SEQ ID NO: 125 - DNA, 13 bp, Artificial Sequence
  Synthetic
aacgatggatggc

SEQ ID NO: 126 - DNA, 13 bp, Artificial Sequence
  Synthetic
tgctatggatggc

SEQ ID NO: 127 - DNA, 13 bp, Artificial Sequence
  Synthetic
gaccatgtatggc

SEQ ID NO: 128 - DNA, 13 bp, Artificial Sequence
  Synthetic
gataatgtatggc

SEQ ID NO: 129 - DNA, 13 bp, Artificial Sequence
  Synthetic
ggctctggatggc